(19) United States
(12) Patent Application Publication
(10) Pub. No.: US 2019/0259473 A1
(43) Pub. Date: Aug. 22, 2019

(54) IDENTIFICATION OF INDIVIDUALS BY TRAIT PREDICTION FROM THE GENOME

(71) Applicants: Franz J. OCH, San Diego, CA (US); M. Cyrus MAHER, San Diego, CA (US); Victor LAVRENKO, San Diego, CA (US); Christoph LIPPERT, San Diego, CA (US); David HECKERMAN, San Diego, CA (US); David SHUTE, San Diego, CA (US); Okan ARIKAN, San DIego, CA (US); Riccardo SABATINI, San Diego, CA (US); Eun Young KANG, San Diego, CA (US); Peter GARST, San Diego, CA (US); Axel BERNAL, San Diego, CA (US); Mingfu ZHU, San Diego, CA (US); Alena HARLEY, San Diego, CA (US); Theodore WONG, San Diego, CA (US); Seunghak LEE, San Diego, CA (US)

(72) Inventors: Franz J. OCH, San Diego, CA (US); M. Cyrus MAHER, San Diego, CA (US); Victor LAVRENKO, San Diego, CA (US); Christoph LIPPERT, San Diego, CA (US); David HECKERMAN, San Diego, CA (US); David SHUTE, San Diego, CA (US); Okan ARIKAN, San DIego, CA (US); Riccardo SABATINI, San Diego, CA (US); Eun Young KANG, San Diego, CA (US); Peter GARST, San Diego, CA (US); Axel BERNAL, San Diego, CA (US); Mingfu ZHU, San Diego, CA (US); Alena HARLEY, San Diego, CA (US); Theodore WONG, San Diego, CA (US); Seunghak LEE, San Diego, CA (US)

(21) Appl. No.: 16/324,463

(22) PCT Filed: Aug. 7, 2017
(86) PCT No.: PCT/US2017/045781

§ 371 Date: Feb. 22, 2019

Related U.S. Application Data
(60) Provisional application No. 62/372,297, filed on Aug. 8, 2016.

(51) Int. Cl. G16B 45/00; G16B 40/00; G16B 5/20; G16B 20/40; G16B 20/20; C12Q 1/6876; G06K 9/00

(52) U.S. Cl. CPC G16B 45/00; G16B 40/00; G16B 5/20; G16B 20/40; G16B 20/20; C12Q 2600/158; G06K 9/00248; C12Q 2600/156; C12Q 2600/154; C12Q 2600/124; C12Q 1/6876

(57) ABSTRACT

Described are methods and systems for identifying phenotypic traits of an individual from nucleotide sequence data. The methods and systems are useful even when the identity of the individual or phenotypic traits of the individual is unknown.

### CROSS-REFERENCE TO RELATED APPLICATIONS

This application claims the benefit of U.S. Application Ser. No. 62/372,297 filed Aug. 8, 2016, the entire contents of which are hereby incorporated by reference.

### BACKGROUND OF THE INVENTION

Much of the promise of whole genome sequencing relies on the ability to associate genotypes to physical traits. Forensic applications include post-mortem identification and the association and identification of DNA from biological evidence for intelligence agencies and federal, state, and local law enforcement. In the United States, an average of approximately 35% of homicides and 60% of sexual assaults remain unsolved. For crimes such as these, DNA evidence, e.g., a spot of blood at a crime scene, may be available. In many cases, the perpetrator's DNA is not included in a database such as the Combined DNA Index System (CODIS).

### SUMMARY

Different forensic models exist for predicting individual traits such as skin color, eye color, and facial structure. However, there is a long-felt and unmet need for the ability to produce highly personalized phenotypic prediction profiles, height, age, weight, and facial structure and demographic information such as age, gender, and race. These methods are limited by narrow focus on specific genetic polymorphisms, and the inability to determine important covariates for facial structure. Described herein are methods that predict multiple phenotypic and demographic traits from a single sample resulting in more efficient and cost effective analysis. Described herein are methods for matching DNA evidence to more commonly available phenotypic sets, such as facial images and basic demographic information, thereby addressing cases where conventional DNA testing, database search, and familial testing fails.

Described herein are predictive models for facial structure, voice, eye color, skin color, height, weight, BMI, age, and blood group using whole genome sequence data. We show that, individually, each of these models provides weak information about an individual's identity. Leveraging our method for forensic model integration, however, we demonstrate the possibility to match genomes to phenotypic profiles such as the data found in online profiles. The methods described herein can improve phenotypic prediction as cohorts continue to grow in size and diversity. It can also integrate information from diverse experimental sources. For example, age prediction from DNA methylation can be combined with the methods described herein to improve performance relative to our purely genome-based approach, and is envisioned by this disclosure.

When no investigative leads are available, the procedures presented here may help define a manageable suspect set, e.g., by querying genomes against Facebook profiles, LinkedIn profiles, images from dating websites or applications, or any image database. Additionally, this method may be used to prioritize suspect lists in order to reduce the time and cost involved in criminal investigations. Further, it could also be used to support the identification of terrorists, as well as victims of crimes, accidents, or disasters.

In another aspect, phenotypic traits can be predicted from a composite genome, the composite genome comprising genetic information from two individuals. This could for example be used to predict the appearance and a child from a mother and father.

In another aspect, the methods described herein can be used to anonymize genomic data so that physical phenotypic traits such as eye color, skin color, hair color, or facial structure cannot be determined. Here, we show that prediction of physical traits from the genome enabled re-identification without relying on any further information being shared. This suggests that genome sequences cannot be considered de-identifiable, and so should be shared only using an appropriate level of security and due diligence. The Health Insurance Portability and Accountability Act (HIPAA) does not currently consider genome sequences as identifying information that has to be removed under the Safe Harbor Method for de-identification. In certain embodiments, the method comprises masking or anonymizing key genomic loci from an individual's genome.

In another aspect described herein is a method of determining a facial structure of an individual from a nucleic acid sequence for the individual, the method comprising: (a) determining a plurality of genomic principal components from the nucleic acid sequence of the individual that are predictive of facial structure; and (b) determining at least one demographic feature from the nucleic acid sequence of the individual selected from the list consisting of: (i) an age of the individual; (ii) a sex of the individual; and (iii) an ancestry of the individual; wherein the facial structure is determined according to the genomic principal components and the at least one demographic feature from the nucleic acid sequence of the individual. In certain embodiments, the method comprises determining at least two demographic features from the nucleic acid sequence selected from the list consisting of (i) an age of the individual; (ii) a sex of the individual; and (iii) an ancestry of the individual. In certain embodiments, the method comprises determining at least all three of (i) an age of the individual; (ii) a sex of the individual; and (iii) an ancestry of the individual from the nucleic acid sequence from the individual. In certain embodiments, the facial structure of the individual is uncertain or unknown at the time of determination. In certain embodiments, the individual is a human. In certain embodiments, the genomic principal components are derived from a data set comprising a plurality of facial structure measurements and a plurality of genome sequences. In certain embodiments, the plurality of genome sequences is at least 1,000 genome sequences. In certain embodiments, the genomic principal components from the nucleic acid sequence that are predictive of facial structure are predictive of facial landmark distance. In certain embodiments, the nucleic acid sequence for the individual was obtained from a biological sample obtained from a crime scene. In certain embodiments, the nucleic acid sequence for the individual is an in silico generated sequence that is a composite of two individuals. In certain embodiments, the biological sample is from the group consisting of blood, urine, feces, semen, hair, skin cells, teeth, and bone. In certain embodiments, the plurality of genomic principal components determine at least 90% of the observed variation of facial structure. In certain embodiments, the age of the individual is determined by both the average telomere length and the mosaic loss of the sex chromosome from the nucleic acid sequence for the individual. In certain embodiments, the average telomere length is determined by a next-generation DNA sequencing method. In certain embodiments, the average telomere length is determined by a proportion of putative telomere reads to total reads. In certain embodiments, the sex chromosome is the Y chromosome if the individual is known or alleged to be a male. In certain embodiments, the mosaic loss of Y chromosome is determined by sequences from the Y chromosome that are Y chromosome specific. In certain embodiments, the sex chromosome is the X chromosome if the individual is known or alleged to be a female. In certain embodiments, the mosaic loss of a sex chromosome is determined by determining chromosomal copy number. In certain embodiments, the mosaic loss of a sex chromosome is determined by a next-generation sequencing method. In certain embodiments, the mean absolute error of the method of determining the age of the individual from the biological sample comprising genomic DNA is equal to or less than 10 years. In certain embodiments, the R_{2CV} of the method of determining the age of the individual from the biological sample comprising genomic DNA is equal to or greater than 0.40. In certain embodiments, the sex of the individual is determined by estimating copy number of the X and Y chromosome. In certain embodiments, the sex of the individual is determined by a next-generation DNA sequencing method. In certain embodiments, the ancestry of the individual is determined by a plurality of single nucleotide polymorphisms that are informative of ancestry. In certain embodiments, the ancestry of the individual is determined by a next-generation DNA sequencing method. In certain embodiments, the method further comprises determining a body mass index of the individual from the biological sample. In certain embodiments, the method further comprises determining the presence or absence of at least one single nucleotide polymorphism associated with facial structure. In certain embodiments, the facial structure determined is a plurality of land mark distances. In certain embodiments, the plurality of land mark distances comprise at least two or more of TGL_TGRpa, TR_GNpa, EXR_ENR (Width of the right eye), PSR_PIR (Height of the right eye), ENR_ENL (Distance from inner left eye to inner right eye), EXL_ENL (Width of the left eye), EXR_EXL (Distance from outer left eye to outer right eye), PSL_PIL (Height of the left eye), ALL_ALR (Width of the nose), N_SN (Height of the nose), N_LS (Distance from top of the nose to top of upper lip), N_ST (Distance from top of the nose to center point between lips), TGL_TGR (Straight distance from left ear to right ear), EBR_EBL (Distance from inner right eyebrow to inner left eyebrow), IRR_IRL (Distance from right iris to left iris), SBALL_SBALR (Width of the bottom of the nose), PRN_IRR (Distance from the tip of the nose to right iris), PRN_IRL (Distance from the tip of the nose to left iris), CPHR_CPHL (Distance separating the crests of the upper lip), CHR_CHL (Width of the mouth), LS_LI (Height of lips), LS_ST (Height of upper lip), LI_ST (Height of lower lip), TR_G (Height of forehead), SN_LS (Distance from bottom of the nose to top of upper lip), LI_PG (Distance from bottom of the lower lip to the chin). In certain embodiments, the plurality of land mark distances comprise ALL_ALR (width of nose) and LS_LI (height of lip). In certain embodiments, the method further comprises generating a graphical representation of the determined facial structure. In certain embodiments, the method further comprises displaying the graphical representation of the determined facial structure. In certain embodiments, the method further comprises transmitting the graphical representation to a 3D rapid prototyping device.

In another aspect described herein, is a computer-implemented system comprising a digital processing device comprising at least one processor, an operating system configured to perform executable instructions, a memory, and a computer program including instructions executable by the digital processing device to create an application comprising: (a) a software module configured to determine a plurality of genomic principal components from the nucleic acid sequence of an individual that are predictive of facial structure; (b) a software module configured to determine at least one demographic feature from the nucleic acid sequence of the individual selected from the list consisting of determining: (i) an age of the individual; (ii) a sex of the individual; and (iii) an ancestry; and (c) a software module configured to determine a facial structure of the individual according to the genomic principal components and the at least one demographic feature from the nucleic acid sequence of the individual. In certain embodiments, the software module determines at least two demographic features from the nucleic acid sequence selected from the list consisting of (i) an age of the individual; (ii) a sex of the individual; and (iii) an ancestry of the individual. In certain embodiments, the software module determines at least all three of (i) an age of the individual; (ii) a sex of the individual; and (iii) an ancestry of the individual from the nucleic acid sequence from the individual. In certain embodiments, the facial structure of the individual is uncertain or unknown at the time of determination. In certain embodiments, the individual is a human. In certain embodiments, the genomic principal components are derived from a data set comprising a plurality of facial structure measurements and a plurality of genome sequences. In certain embodiments, the plurality of genome sequences is at least 1,000 genome sequences. In certain embodiments, the genomic principal components from the nucleic acid sequence that are predictive of facial structure are predictive of facial landmark distance. In certain embodiments, the nucleic acid sequence for the individual was obtained from a biological sample obtained from a crime scene. In certain embodiments, the nucleic acid sequence for the individual is an in silico generated sequence that is a composite of two individuals. In certain embodiments, the biological sample is from the group consisting of blood, urine, feces, semen, hair, skin cells, teeth, and bone. In certain embodiments, the plurality of genomic principal components determine at least 90% of the observed variation of facial structure. In certain embodiments, the age of the individual is determined by both the average telomere length and the mosaic loss of the sex chromosome from the nucleic acid sequence for the individual. In certain embodiments, the average telomere length is determined by a next-generation DNA sequencing method. In certain embodiments, the average telomere length is determined by a proportion of putative telomere reads to total reads. In certain embodiments, the sex chromosome is the Y chromosome if the individual is known or alleged to be a male. In certain embodiments, the mosaic loss of Y chromosome is determined by sequences from the Y chromosome that are Y chromosome specific. In certain embodiments, the sex chromosome is the X chromosome if the individual is known or alleged to be a female. In certain embodiments, the mosaic loss of a sex chromosome is determined by determining chromosomal copy number. In certain embodiments, the mosaic loss of a sex chromosome is determined by a next-generation sequencing method. In certain embodiments, the mean absolute error of the method of determining the age of the individual from the biological sample comprising genomic DNA is equal to or less than 10 years. In certain embodiments, the R²_{CV} of the method of determining the age of the individual from the biological sample comprising genomic DNA is equal to or greater than 0.40. In certain embodiments, the sex of the individual is determined by estimating copy number of the X and Y chromosome. In certain embodiments, the sex of the individual is determined by a next-generation DNA sequencing method. In certain embodiments, the ancestry of the individual is determined by a plurality of single nucleotide polymorphisms that are informative of ancestry. In certain embodiments, the ancestry of the individual is determined by a next-generation DNA sequencing method. In certain embodiments, the system further comprises a software module configured to determine a body mass index of the individual from the biological sample. In certain embodiments, the system further comprises a software configured module to determine the presence or absence of at least one single nucleotide polymorphism associated with facial structure. In certain embodiments, the facial structure determined is a plurality of land mark distances. In certain embodiments, the plurality of land mark distances comprise at least two or more of TGL_TGRpa, TR_GNpa, EXR_ENR (Width of the right eye), PSR_PIR (Height of the right eye), ENR_ENL (Distance from inner left eye to inner right eye), EXL_ENL (Width of the left eye), EXR_EXL (Distance from outer left eye to outer right eye), PSL_PIL (Height of the left eye), ALL_ALR (Width of the nose), N_SN (Height of the nose), N_LS (Distance from top of the nose to top of upper lip), N_ST (Distance from top of the nose to center point between lips), TGL_TGR (Straight distance from left ear to right ear), EBR_EBL (Distance from inner right eyebrow to inner left eyebrow), IRR_IRL (Distance from right iris to left iris), SBALL_SBALR (Width of the bottom of the nose), PRN_IRR (Distance from the tip of the nose to right iris), PRN_IRL (Distance from the tip of the nose to left iris), CPHR_CPHL (Distance separating the crests of the upper lip), CHR_CHL (Width of the mouth), LS_LI (Height of lips), LS_ST (Height of upper lip), LI_ST (Height of lower lip), TR_G (Height of forehead), SN_LS (Distance from bottom of the nose to top of upper lip), LI_PG (Distance from bottom of the lower lip to the chin). In certain embodiments, the plurality of land mark distances comprise ALL_ALR (width of nose) and LS_LI (height of lip). In certain embodiments, the system further comprises a software module configured to generate a graphical representation of the determined facial structure. In certain embodiments, the system further comprises a software module configured to display the graphical representation of the determined facial structure. In certain embodiments, the system further comprises a software module configured to transmit the graphical representation to a 3D rapid prototyping device.

In another aspect, disclosed herein, is a method of determining an age of an individual from a biological sample comprising genomic DNA from the individual, the method comprising: (a) determining an average telomere length of the genomic DNA from the biological sample; and (b) determining a mosaic loss of a sex chromosome of the genomic DNA from the biological sample; wherein the age of the individual is determined by both the average telomere length and the mosaic loss of the sex chromosome of the genomic DNA from the biological sample. In certain embodiments, the age of the individual is uncertain at the time of determination. In certain embodiments, the individual is a human. In certain embodiments, the biological sample was obtained from a crime scene. In certain embodiments, the biological sample is from the group consisting of blood, urine, feces, semen, hair, skin cells, teeth, and bone. In certain embodiments, the average telomere length is determined by a next-generation DNA sequencing method. In certain embodiments, the average telomere length is determined by a proportion of putative telomere reads to total reads. In certain embodiments, the sex of the individual is determined prior to the determination of the age of the individual. In certain embodiments, the sex chromosome is the Y chromosome if the individual is known or alleged to be a male. In certain embodiments, the mosaic loss of Y chromosome is determined by sequences from the Y chromosome that are Y chromosome specific. In certain embodiments, the sex chromosome is the X chromosome if the individual is known or alleged to be a female. In certain embodiments, the mosaic loss of a sex chromosome is determined by determining chromosomal copy number. In certain embodiments, the mosaic loss of a sex chromosome is determined by a next-generation sequencing method. In certain embodiments, the mean absolute error of the method of determining the age of the individual is equal to or less than 10 years.

In another aspect, disclosed herein, is a method of determining a height of an individual from a biological sample comprising genomic DNA from the individual, the method comprising: (a) determining a plurality of genomic principal components from the biological sample that are predictive of height; and (b) determining a sex of the individual from the biological sample; wherein the height of the individual is determined by the genomic principal components and the sex of the individual. In certain embodiments, the height of the individual is uncertain at the time of determination. In certain embodiments, the individual is a human. In certain embodiments, the biological sample was obtained from a crime scene. In certain embodiments, the biological sample is from the group consisting of blood, urine, feces, semen, hair, skin cells, teeth, and bone. In certain embodiments, the genomic principal components are derived from a data set comprising a plurality of height measurements and a plurality of genome sequences. In certain embodiments, the plurality of genomic principal components are determined from at least 1000 genomes. In certain embodiments, the plurality of genomic principal components summarize at least 90% of the observed variation of height In certain embodiments, the sex of the individual is determined by estimating copy number of the X and Y chromosome. In certain embodiments, the sex of the individual is determined by a next-generation DNA sequencing method. In certain embodiments, the method further comprises determining the presence or absence of at least one single nucleotide polymorphism that is predictive of height. In certain embodiments, the R²_{CV} of the method of determining the height of the individual is equal to or greater than 0.50. In certain embodiments, the method further comprises creating a scaled graphical representation of the individual's height. In certain embodiments, the method further comprises displaying a scaled graphical representation of the individual's height.

In another aspect, disclosed herein, is a method of determining a body mass index of an individual from a biological sample comprising genomic DNA from the individual, the method comprising: (a) determining a plurality of genomic principal components from the biological sample that are predictive of body mass index; (b) determining an age of the individual from the biological sample; and (c) determining a sex of the individual from the biological sample; wherein the body mass index of the individual is determined by the genomic principal components, the age, and the sex of the individual. In certain embodiments, the body mass index of the individual is uncertain at the time of determination. In certain embodiments, the individual is a human. In certain embodiments, the biological sample was obtained from a crime scene. In certain embodiments, the biological sample is from the group consisting of blood, urine, feces, semen, hair, skin cells, teeth, and bone. In certain embodiments, the genomic principal components are derived from a data set comprising a plurality of body mass index measurements and a plurality of genome sequences. In certain embodiments, the plurality of genomic principal components are determined from at least 1000 genomes. In certain embodiments, the plurality of genomic principal components summarize at least 90% of the total variation of body mass index. In certain embodiments, the age of the individual is determined by both the average telomere length and the mosaic loss of the sex chromosome of the genomic DNA from the biological sample. In certain embodiments, the average telomere length is determined by a next-generation DNA sequencing method. In certain embodiments, the average telomere length is determined by a proportion of putative telomere reads to total reads. In certain embodiments, the sex chromosome is the Y chromosome if the individual is known or alleged to be a male. In certain embodiments, the mosaic loss of Y chromosome is determined by sequences from the Y chromosome that are Y chromosome specific. In certain embodiments, the sex chromosome is the X chromosome if the individual is known or alleged to be a female. In certain embodiments, the mosaic loss of a sex chromosome is determined by determining chromosomal copy number. In certain embodiments, the mosaic loss of a sex chromosome is determined by a next-generation sequencing method. In certain embodiments, the mean absolute error of the method of determining the age of the individual from the biological sample comprising genomic DNA is equal to or less than 10 years. In certain embodiments, the R²_{CV} of the method of determining the age of the individual from the biological sample comprising genomic DNA is equal to or greater than 0.40. In certain embodiments, the sex of the individual is determined by estimating copy number of the X and Y chromosome. In certain embodiments, the sex of the individual is determined by a next-generation DNA sequencing method. In certain embodiments, the method further comprises determining the presence or absence of at least one single nucleotide polymorphism that is predictive of body mass index. In certain embodiments the method further comprises determining the height of an individual by a method comprising: (a) determining a plurality of genomic principal components from the biological sample that are predictive of height, wherein the genomic principal components are derived from a data set comprising a plurality of height measurements and a plurality of genome sequences; and (b) determining a sex of the individual from the biological sample; wherein the height of the individual is determined by the genomic principal components and the sex of the individual. In certain embodiments, the method of determining the body mass index of the individual is equal to or greater than 0.10. In certain embodiments, the method further comprise creating a scaled graphical representation of the individual's body mass index. In certain embodiments, the method further comprise displaying a scaled graphical representation of the individual's body mass index.

In another aspect, disclosed herein, is a method of determining an eye color of an individual from a biological sample comprising genomic DNA from the individual, the method comprising: determining a plurality of genomic principal components from the biological sample that are predictive of eye color; wherein the body mass index of the individual is determined by the genomic principal components of the individual. In certain embodiments, the eye color of the individual is uncertain at the time of determination. In certain embodiments, the individual is a human. In certain embodiments, the biological sample was obtained from a crime scene. In certain embodiments, the biological sample is from the group consisting of blood, urine, feces, semen, hair, skin cells, teeth, and bone. In certain embodiments, the genomic principal components are derived from a data set comprising a plurality of eye color measurements and a plurality of genome sequences. In certain embodiments, the plurality of genomic principal components are determined from at least 1000 genomes. In certain embodiments, the plurality of genomic principal components summarize at least 90% of the observed variation of eye color. In certain embodiments, the method further comprises determining the presence or absence of at least one single nucleotide polymorphism that is predictive of eye color. In certain embodiments, the R²_{CV} of the method of determining the eye color of the individual is equal to or greater than 0.7. In certain embodiments, the method further comprises generating a colored graphical representation of the determined eye color. In certain embodiments, the method further comprises displaying the colored graphical representation of the determined eye color.

In another aspect, disclosed herein, is a method of determining a skin color of an individual from a biological sample comprising genomic DNA from the individual, the method comprising: determining a plurality of genomic principal components from the biological sample that are predictive of skin color; wherein the skin color is determined by the genomic principal components of the individual. In certain embodiments, the skin color of the individual is uncertain at the time of determination. In certain embodiments, the individual is a human. In certain embodiments, the biological sample was obtained from a crime scene. In certain embodiments, the biological sample is from the group consisting of blood, urine, feces, semen, hair, skin cells, teeth, and bone. In certain embodiments, the genomic principal components are derived from a data set comprising a plurality of skin color measurements and a plurality of genome sequences In certain embodiments, the plurality of genomic principal components are determined from at least 1000 genomes. In certain embodiments, the plurality of genomic principal components summarize at least 90% of the observed variation of skin color. In certain embodiments, the method further comprises determining the presence or absence of at least one single nucleotide polymorphism that is predictive of skin color. In certain embodiments, the R²_{CV} of the method of determining the skin color of the individual is equal to or greater than 0.7. In certain embodiments, the method further comprises generating a colored graphical representation of the determined skin color. In certain embodiments, the method further comprises displaying the colored graphical representation of the determined skin color.

In another aspect, disclosed herein, is a method of determining a voice pitch of an individual from a biological sample comprising genomic DNA from the individual the method comprising: (a) determining a plurality of genomic principal components from the biological sample that are predictive of voice, wherein the genomic principal components are derived from a data set comprising a plurality of voice pitch measurements and a plurality of genome sequences; (b) determining a sex of the individual from the biological sample; and wherein the voice pitch is determined by the genomic principal components, and the sex from the biological sample of the individual. In certain embodiments, the voice pitch of the individual is uncertain at the time of determination. In certain embodiments, the individual is a human. In certain embodiments, the biological sample was obtained from a crime scene. In certain embodiments, the biological sample is from the group consisting of blood, urine, feces, semen, hair, skin cells, teeth, and bone. In certain embodiments, the plurality of genomic principal components are determined from at least 1000 genomes. In certain embodiments, the plurality of genomic principal components summarize at least 90% of the observed variation of voice pitch. In certain embodiments, the sex of the individual is determined by estimating copy number of the X and Y chromosome. In certain embodiments, the sex of the individual is determined by a next-generation DNA sequencing method. In certain embodiments, the R²_{CV} of the method of determining the voice pitch of the individual is equal to or greater than 0.7. In certain embodiments, the method further comprises generating an audio file of the determined voice pitch. In certain embodiments, the method further comprises transmitting the audio file to an audio playback device. In certain embodiments, the method further comprises playing the audio file of the determined voice pitch.

### BRIEF DESCRIPTION OF THE DRAWINGS

FIGS. 1A-1C illustrate the joint distribution of sex and inferred genomic ancestry in the study population; (A) each person was considered to belong to a given ancestry group if the corresponding inferred ancestry component exceeded 70%, and otherwise was considered admixed. Ancestries are African (AFR), Native American (AMR), Central South Asian (CSA), East Asian (EAS), and European (EUR). (B) Illustrates the distribution of ages in the study. (C) Illustrates the inferred genomic ancestry proportions for each study participant.

FIG. 2 shows an overview of the experimental approach. A variety of phenotypes are collected for each individual, those phenotypes are then predicted from the genome, and the concordance between predicted and observed are used to match an individual's phenotypic profile to the genome.

FIG. 3 illustrates facial landmarks overlaid on a facial image.

FIGS. 4A-C illustrate alignment of 3D scan of face images to the template face model. To minimize the noise due to face image misalignment between different face samples, we aligned face 3D images by matching the vertex of the average template face and each individual face. (A) The vertices of the average template face and their normal vectors. (B) Gray vertices represent the vertex in the average template. Red solid lines represent the scanned face surface for the observed samples. (C) Average face template vertices are displaced along their normal vectors to the closest observed scanned surface. If there is no scanned surface near the template vertices, the closest scanned surfaces are estimated using Poisson method.

FIGS. 5A and 5B illustrate automatic extraction of the iris area from 2D eye images. (A) An eye image extracted from a face image. (B) Blue area showing the identified iris by the proposed iris extraction method.

FIG. 6 illustrates the three skin patches (rectangular regions) used for skin color estimation superimposed onto an albedo normalized face image.

FIG. 7 illustrates a pipeline for i-vector generation.

FIG. 8 illustrates the distributions for chromosomal copy number (CCN) for chrX vs chrY computed for all the samples in our dataset.

FIG. 9 illustrates predicted versus true age, R²_{CV} for models using features including telomere length (telomeres), and X and Y copy number (X/Y copy).

FIGS. 10A-10D illustrate regression plots for telomere length and X or Y chromosomal copy number against age showing correlation between true age and variables including (A) telomere length, (B) chromosome X copy number, and (C) chromosome Y copy number. (D) Also shown are held out predictions vs real age for all samples.

FIGS. 11A and 11B illustrate correlation between weighted sum of GIANT SNP factor and the observed (A) male height and (B) female height.

FIGS. 12A-12D illustrate a correlation plot between predicted height and observed height with different features and cross validated with 4082 individuals (A) Age; (B) Age+Sex; (C) Age+Sex+100PCs; (D) Age+Sex+100PCs+SNP Height (696 height associated SNPs).

FIG. 13A-13D illustrate a correlation plot between predicted BMI and observed BMI with different features in 10 cross validation with 4082 individuals. (A) Age; (B) Age+Sex; (C) Age+Sex+100PCs; (D) Age+Sex+100PCs+SNP_BMI.

FIGS. 14A-14E illustrate a correlation between predicted weight and observed weight with different features. (A) Age; (B) Age+Sex; (C) Age+Sex+100PCs; (D) Age+Sex+100PCs+SNP_Height; (E) Age+Sex+100PCs+SNP_Height+SNP_BMI.

FIGS. 15A-15C illustrate predictive performance for eye color. (A) PCA projection of observed eye color, (B) the correlation between the first PC of observed values and the first PC of predicted values, (C) and predictive performance of models using different covariate sets composed from three genomic PCs and previously reported SNPs.

FIGS. 16A-16C illustrate predictive performance for skin color. (A) PCA projection of observed skin color, (B) the correlation between the first PC of observed values and the first PC of predicted values, (C) and cross-validated variance explained by models using different covariate sets composed from three genomic PCs and previously reported SNPs.

FIG. 17 illustrates observed (top circle) and predicted (bottom circle) skin colors for 1,022 individuals using our best performing model (Extreme Boosted Tree), 3 first PCs, predicted age, predicted gender, and 7 SNPs.

FIGS. 18A-18W illustrate a holdout set of 24 individuals. Left most face, true face; middle face, Ridge Regression predicted face; right most face, Ridge for Depth PCs, k-Nearest Neighbor for Color PCs.

FIGS. 19A-19C illustrate scan vs. 3D prediction for three selected individuals from the holdout set. Top row in each panel represents observed face (0 degree, 45 degree and 90 degree rotated), and bottom row in each panel represents predicted face (0 degree, 45 degree and 90 degree rotated).

FIG. 20 illustrates the performance of face prediction. Shown is per-pixel R²_{CV} as a function of model features, presented for the horizontal, vertical and depth axes. The models have been trained on combinations of: sex, ancestry-defining genome PCs (Anc), and reported SNPs (SNPs), true age (Age) and BMI.

FIG. 21 illustrates Per-pixel R²_{CV} for the full model, across three axes.

FIG. 22A-22B illustrate quantile-quantile (QQ) plots for association tests between all tests of 36 candidate SNPs vs. top 10 PCs for face color data and top 10 PCs for face depth data. (A) Association statistics are computed using age gender and BMI as covariates, and (B) association statistics are computed using age, gender, BMI, and 5 ethnicity proportions (AFR, EUR, EAS, CEA, AMR) as covariates. Comparison of these QQ plots shows that these 36 previously identified SNPs are highly correlated with ethnicity.

FIG. 23 shows landmark distance predictions. The measured performance in R²_{CV} (observed vs. predicted) of predicted landmark distances using sex, predicted age, and top 3 genome PCs. ALL_ALR (the width of the flaring of the nostril) is the highest performing landmark in our study.

FIG. 24 illustrates a schematic representation of the difference between select optimization (best option chosen independently) and match optimization (globally optimal edge set chosen). Select corresponds to picking an individual out of a group of individuals based on a genomic sample. Match corresponds to post-mortem identification of groups of individuals.

FIG. 25 illustrates the top one accuracy in match and select. Average accuracy in select and match for different pool sizes from 2 to 50 using various features. Random performance is shown in grey.

FIG. 26 illustrates ranking performance. The empirical probability that the true subject is ranked in the top N as a function of the pool size. Solid lines represent performance with the current features set.

FIG. 27 illustrates match and select accuracy. Accuracy for matching the PGP10 individuals to their genomes (m₁₀) and accuracy for selecting the correct individual from the PGP10 given a genome (s₁₀).

FIG. 28 shows a graph representation of genotype and phenotype similarities. A force-directed representation of the similarities between genotypes (purple) and phenotypes (yellow). Red edges represent mismatching genotype/phenotype pairs, while green edges illustrate matches. Edge width conveys the similarity between linked nodes. Numbers correspond to participant identification codes. For example, PGP-1 is 1. For both m₁₀ and s₁₀, all ten individuals are matched correctly (right).

FIGS. 29A and 29B illustrate the performance of closed-set identification using observed and predicted 2D face image embeddings (NN: neural network based embedding, PC: principle components) on (A) our dataset and (B) PGP dataset.

FIGS. 30A-30J illustrate predictions on PGP-1 to PGP-10 individuals for traits including face, eye color, skin color, surname, age, height, blood type, and ethnicity from genomic features.

FIG. 31 illustrates histograms of R²_{CV} between observed and predicted 2D face images using OpenFace neural network embedding and PC embedding. The green histogram illustrates the prediction performance for 300 principle components representing a 2D face (green). The blue histogram illustrates the prediction performance for the 128 components of the OpenFace neural network embedding.

FIGS. 32A and 32B illustrate (A) m₁₀ and (B) s₁₀ performance comparison between the optimal distance determined using YASMET and the cosine distance on different combinations of phenotypes. In the x-axis, “Demogr.” represents the combined ancestry, age, and gender, “Add'l” represents the combined voice and height/weight/BMI, “All Face” represents the combined 3D face, landmarks, eye color, and skin color, and “Full” represents the combined sets of phenotypes including “Demogr.”, “Add'l”, and “All Face”.

FIG. 33A illustrates s₁₀ as a function of R² for a single trait. The plot shows simulation results for a single independently Gaussian distributed trait as a function of expected R² (blue solid line). A random prediction (green dashed line) would achieve a s₁₀ performance of 10%.

FIG. 33B illustrates s₁₀ performance as a function of number traits. The plot shows how s₁₀ performance changes as a function of the number of traits for different expected R². Random predictions (green dashed line) would achieve a s₁₀ performance of 10% irrespective of the number of traits.

FIG. 34 illustrates an example algorithm for creating a composite genome from two different genomes where the relevant principal components that predict a phenotypic trait are averaged.

FIG. 35 illustrates an example algorithm for creating a composite genome from two different genomes where SNPS that predict a phenotypic trait are chosen from each parent in a stochastic manner.

FIG. 36 illustrates an example algorithm for creating a composite genome from two different genomes where meiotic breakpoints and linkage disequilibrium are assumed for genomic sequences that predict a phenotypic trait.

FIG. 37 illustrates an example user interface for an application that creates a composite genome and predicts a phenotypic feature.

FIG. 38 shows a non-limiting example of a digital processing device; in this case, a device with one or more CPUs, a memory, a communication interface, and a display.

FIG. 39 shows a non-limiting example of a web/mobile application provision system; in this case, a system providing browser-based and/or native mobile user interfaces.

FIG. 40 shows a non-limiting example of a cloud-based web/mobile application provision system; in this case, a system comprising an elastically load balanced, auto-scaling web server and application server resources as well synchronously replicated databases.

### DETAILED DESCRIPTION OF THE INVENTION

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

In one aspect described herein is a method of determining a phenotypic or demographic trait of an individual from a nucleic acid sequence for the individual, the method comprising: (a) determining a plurality of genomic principal components from the nucleic acid sequence that are predictive of the phenotypic or demographic trait. The phenotypic traits predicted by the currently described systems and methods can comprise any one or more of age, height, weight, BMI, eye color, skin color, voice pitch or facial structure. In certain embodiments, any two or more of age, height, weight, BMI, eye color, skin color, voice pitch or facial structure can be predicted. In certain embodiments, any three or more of age, height, weight, BMI, eye color, skin color, voice pitch or facial structure can be predicted.

In another aspect described herein is a method of determining a facial structure of an individual from a nucleic acid sequence for the individual, the method comprising: (a) determining a plurality of genomic principal components from the nucleic acid sequence of the individual that are predictive of facial structure; and (b) determining at least one demographic feature from the nucleic acid sequence of the individual selected from the list consisting of: (i) an age of the individual; (ii) a sex of the individual; and (iii) an ancestry of the individual; wherein the facial structure is determined according to the genomic principal components and the at least one demographic feature from the nucleic acid sequence of the individual.

In another aspect described herein, is a computer-implemented system comprising a digital processing device comprising at least one processor, an operating system configured to perform executable instructions, a memory, and a computer program including instructions executable by the digital processing device to create an application comprising: (a) a software module configured to determine a plurality of genomic principal components from the nucleic acid sequence of an individual that are predictive of facial structure; (b) a software module configured to determine at least one demographic feature from the nucleic acid sequence of the individual selected from the list consisting of determining: (i) an age of the individual; (ii) a sex of the individual; and (iii) an ancestry; and (c) a software module configured to determine a facial structure of the individual according to the genomic principal components and the at least one demographic feature from the nucleic acid sequence of the individual.

When predicting facial structure facial landmark distances can be predicted, and these distances can inform a graphical representation of a given individual's facial structure. In certain embodiments any one, two, three, four, five, six, seven, eight, nine, ten or more of TGL_TGRpa, TR_GNpa, EXR_ENR (Width of the right eye), PSR_PIR (Height of the right eye), ENR_ENL (Distance from inner left eye to inner right eye), EXL_ENL (Width of the left eye), EXR_EXL (Distance from outer left eye to outer right eye), PSL_PIL (Height of the left eye), ALL_ALR (Width of the nose), N_SN (Height of the nose), N_LS (Distance from top of the nose to top of upper lip), N_ST (Distance from top of the nose to center point between lips), TGL_TGR (Straight distance from left ear to right ear), EBR_EBL (Distance from inner right eyebrow to inner left eyebrow), IRR_IRL (Distance from right iris to left iris), SBALL_SBALR (Width of the bottom of the nose), PRN_IRR (Distance from the tip of the nose to right iris), PRN_IRL (Distance from the tip of the nose to left iris), CPHR_CPHL (Distance separating the crests of the upper lip), CHR_CHL (Width of the mouth), LS_LI (Height of lips), LS_ST (Height of upper lip), LI_ST (Height of lower lip), TR_G (Height of forehead), SN_LS (Distance from bottom of the nose to top of upper lip), LI_PG (Distance from bottom of the lower lip to the chin) can be predicted. In certain embodiments, the facial land mark distance predicted can comprise at least ALL_ALR (width of nose) and LS_LI (height of lip). In certain embodiments, the facial land mark distance predicted can comprise at least ALL_ALR and LS_LI; and one, two, three, four, five, six, seven, eight, nine, ten or more of TGL_TGRpa, TR_GNpa, EXR_ENR, PSR, ENR_ENL, EXL_ENL, EXR_EXL, PSL_PIL, N_SN, N_LS, TGL_TGR, EBR_EBL, IRR_IRL, SBALL_SBALR, PRN_IRR, PRN_IRL, CPHR_CPHL, CHR_CHL, LS_ST, LI_ST, TR_G, SN_LS, LI_PG.

In certain embodiments, phenotypic traits are predicted from genomic principal coordinates (PCs) that are derived from a plurality of phenotypic or facial structure measurements (e.g., landmarks) and genome sequences. The measurements are associated with sequence data and the principal coordinates that determine a given measurement are extracted from the nucleic acid sequence data. In certain embodiments, the PCs that are used to predict a phenotype are the top PCs associates with a measurement. In certain embodiments, the top PCs are the top 1, 2, 3, 4, 5, 6, 7, 8, 9, or 10 PCs that determine a measurement for the given feature. In certain embodiments, the top PCs are the top 10, 20, 30, 40, 50, 60, 70, 80, 90, or 100 PCs that determine a measurement for the given feature. In certain embodiments, when predicting facial structure the top PCs that determine facial measurements are combined with one or more other determined phenotypic traits selected from the group consisting of age, height, and ancestry. In certain embodiments, when predicting facial structure the top PCs that determine facial measurements are combined with two or more other determined phenotypic traits selected from the group consisting of age, height, and ancestry. In certain embodiments, when predicting facial structure the top PCs that determine facial measurements are combined with all three determined phenotypic traits selected from the group consisting of age, height, and ancestry. In certain embodiments, the PCs can be combined with a one, two, three, four, five, six or more SNPs predicative of a given trait or landmark measurement. In certain embodiments, the predication of given landmark is accurate to a R²_{CV} value of at least 0.4, 0.5, 0.6, 0.7, 0.8, or 0.9. In certain embodiments, the method predicts an ALL_ALR measurement to an R²_{CV} value of at least 0.4, 0.5, 0.6, 0.7, 0.8, or 0.9. In certain embodiments, the method predicts an LS_LI measurement to an R²_{CV} value of at least 0.4, 0.5, 0.6, 0.7, 0.8, or 0.9.

The methods and systems described herein are useful in predicting various phenotypic characteristics based solely on nucleic acid sequence data. The nucleic acid sequence data can be collected by any method that provides sufficient nucleotide data to allow prediction of a phenotypic trait. For example, facial structure prediction requires a more detailed set of data than prediction of ancestry, eye-color or skin color. In certain aspects, the sequence data is obtained from a next generation sequencing technique, such as, sequencing by synthesis. In other aspects, the sequence data is obtained by SNP mapping of a sufficient number of SNPs to predict a particular trait. The nucleic acid sequence data can comprise a whole-genome, a partial genome, high-confidence regions of the genome, or exome sequence. RNA-Seq data, SNP sequence data (for example acquired from Ancestry.com or 23andme). The nucleic acid sequence can be conveyed in text format, FASTA format, FASTQ format, as a .vcf file, a .bam file, or a .sam file. The nucleic acid sequence data can be DNA sequence data.

In one aspect, the methods and systems described herein are useful for forensic analysis. By predicting phenotypic traits from nucleic acid samples, one can generate a hypothetical suspect or a facial structure useful for identifying an unidentified individual. This individual could, for example, be a suspect of a crime, an unidentified corpse that lacks a head or identifiable facial features or other phenotypic traits. Nucleic acids, primarily DNA, can be extracted from a biological sample of the unknown individual. The biological sample can be from a crime scene or suspected crime scene. The biological sample can comprise a blood sample, a blood spot, teeth, bone, hair, skin cells, saliva, urine, fecal matter, semen, vaginal flood, or a severed appendage (e.g., finger, hand, toe, foot, leg, arm, torso, or penis). Methods of extracting and sequencing DNA from forensic samples are well known in the art, and any appropriate method can be used that yields DNA sufficient for analysis. The amount of DNA does not necessarily need to be enough to conduct full genome sequencing, but can be enough to conduct analysis of a certain amount of SNPs sufficient for trait prediction. In certain embodiments, a facial structure predicted from a DNA sequence is used to query a database of images of suspects. In certain embodiments, the method can identify an individual from a suspect database of greater 100, 200, 300, 400, 500, 600, 700, 800, 900, 1000, 10×10⁴, or 10×10⁵ individual's with at least 90%, 95%, 96%, 97%, 98%, or 99% confidence.

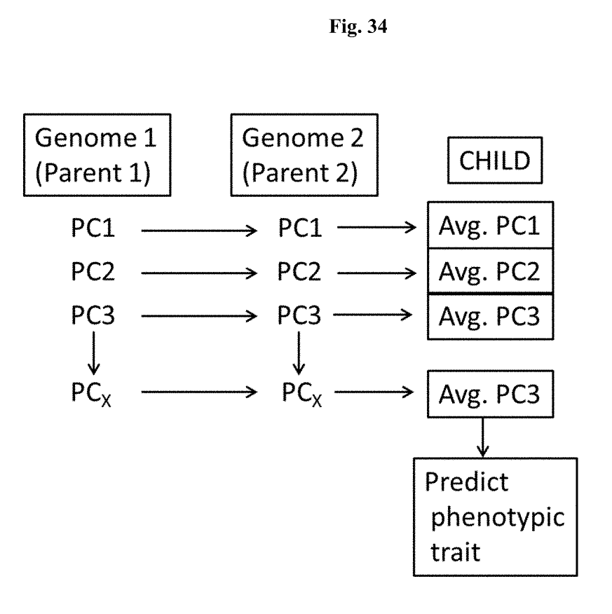

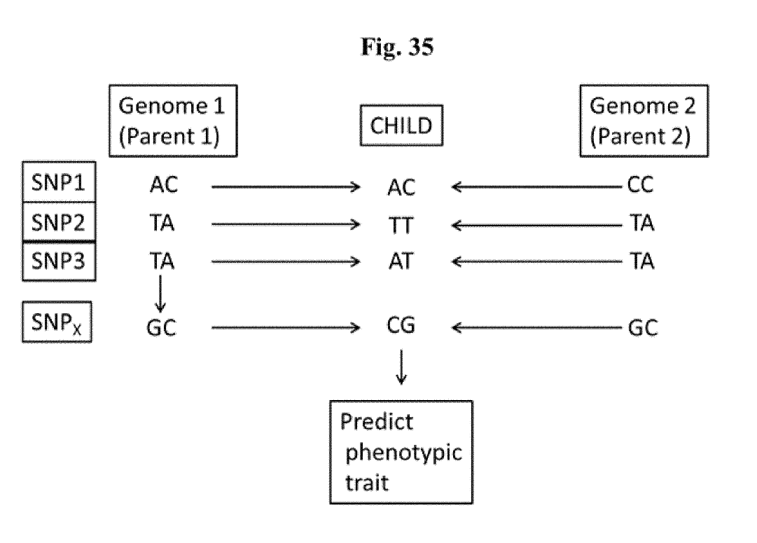

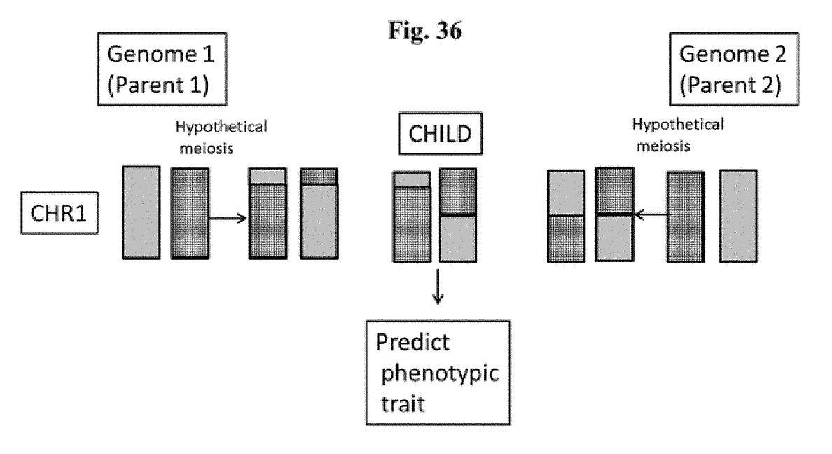

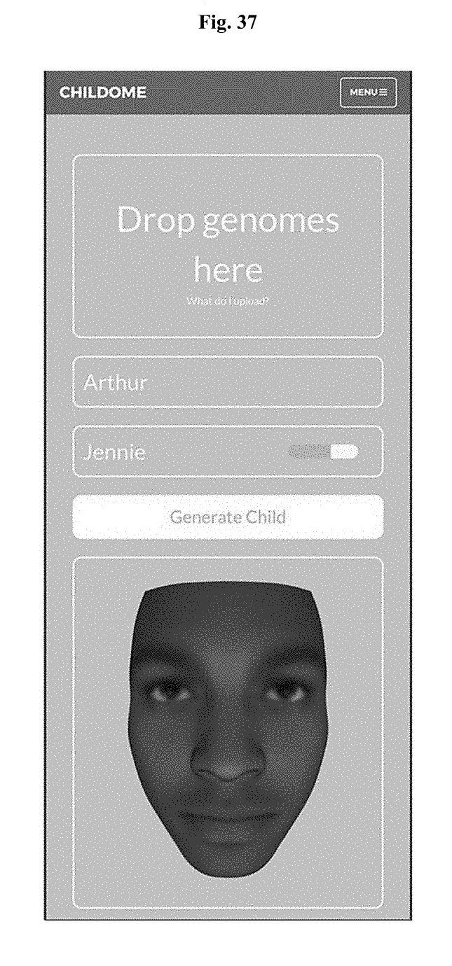

In another aspect, the methods and systems described herein are useful for the predication of phenotypic data from composite genomes. For example, a composite genome can be created from two individuals that have had their genome sequenced or SNP profile determined. This composite genome can be a hypothetical child and the phenotypic data predicted can be, height at a given age, weight at a given age, BMI at a given age, facial structure at a given age, skin color at a given age, eye color at a given age, voice pitch at a given age, height at full maturity, weight at full maturity, BMI at full maturity, skin color, eye color, voice pitch at full maturity or facial structure at full maturity. The two individuals can be two males, two females, or a male and a female. The composite genome can be created in silica from the nucleic acid sequence data of the two individuals. In certain embodiments, the composite genome is information defining the genomic principal coordinates that control certain phenotypic characteristics. For example as shown in FIG. 34, a mean principal component is imputed to the composite genome. These averaged principal components are then utilized to predict a desired phenotypic trait. In FIG. 35, a composite genome is created by collecting SNPs for two individuals and randomly choosing one allele from each individual at each SNP location and imputing that to the composite genome. The SNPs are then used to predict a desired phenotypic trait. Since SNPs are assigned from each individual to the hypothetical child randomly the composite genome can be rendered multiple times, resulting in several slightly different faces. Finally, as shown in FIG. 36 meiosis can be simulated using known common meiotic breakpoints. This creates an “in silica meiosed” genome for each of the two individuals (disregarding sex chromosomes). Then one of the two meiosed chromosomes can randomly be imputed to the hypothetical child and utilized to predict a desired phenotypic trait. This method, however, requires phased genomic data. FIG. 37 show a user interface for a computer/mobile device application that allows a user to input two genomes and predict a hypothetical child. Depending upon the device, the upload prompt can prompt a user to, for example, “drag genomes” to the box, “browse for genome”, or “tap to upload”.

In certain embodiments, the method and systems described herein can be used to display or transmit a graphical representation of the facial structure of the individual. This graphical representation can also predict a simulated age, skin color and eye color of the individual. The representation can be transmitted over a computer network or as a hard copy through the mail.

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX*, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

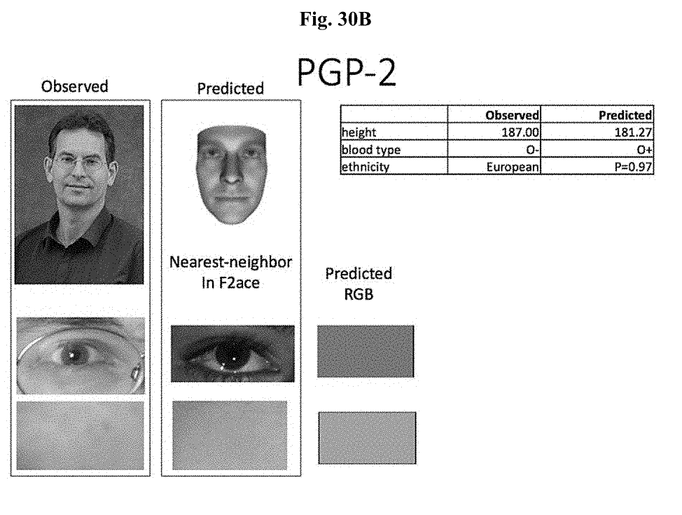

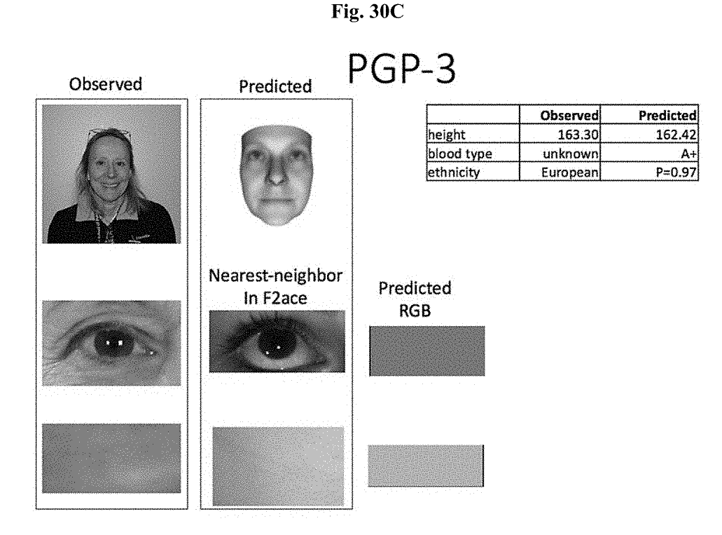

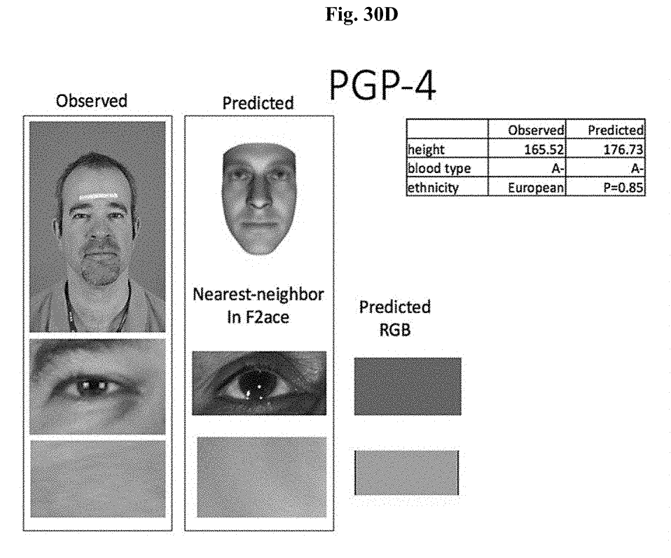

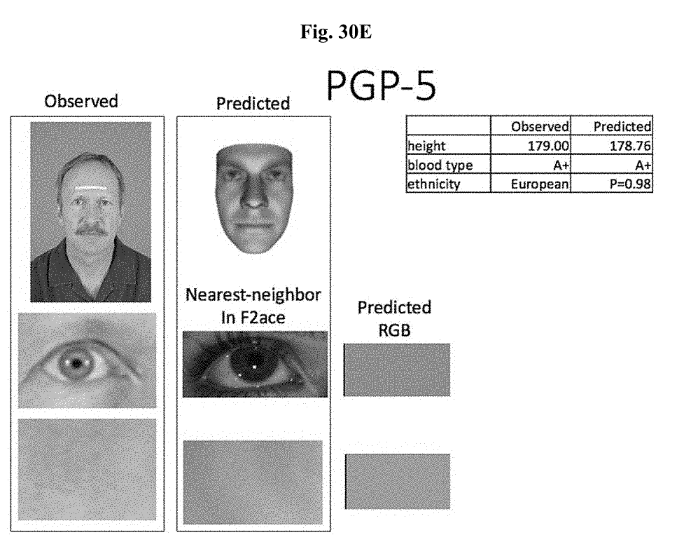

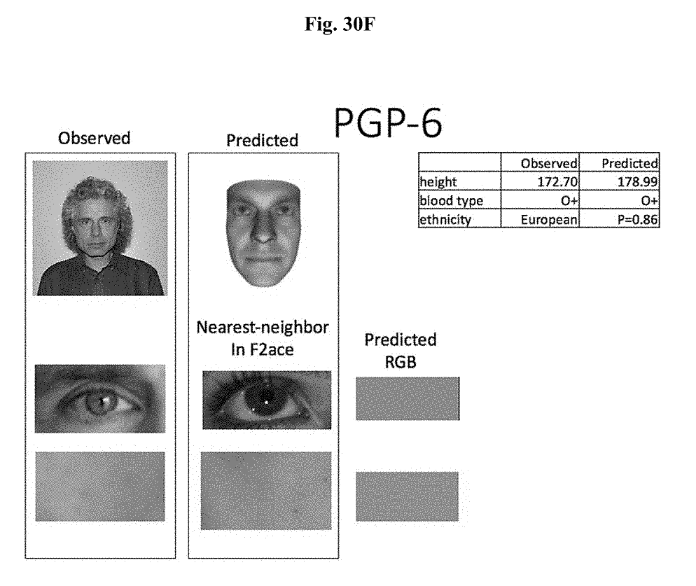

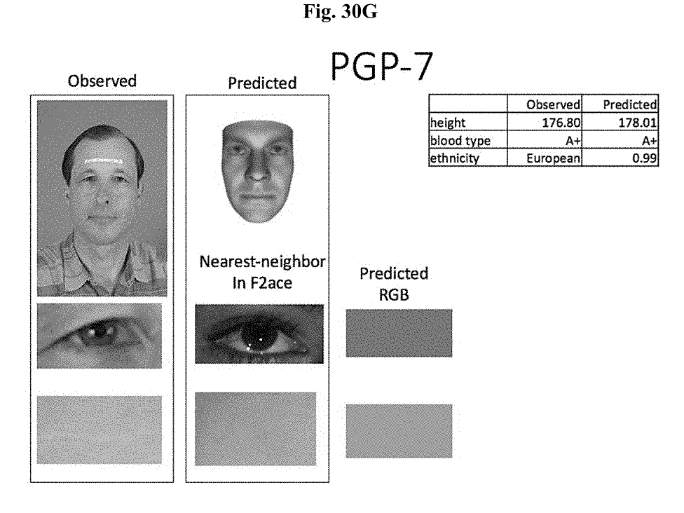

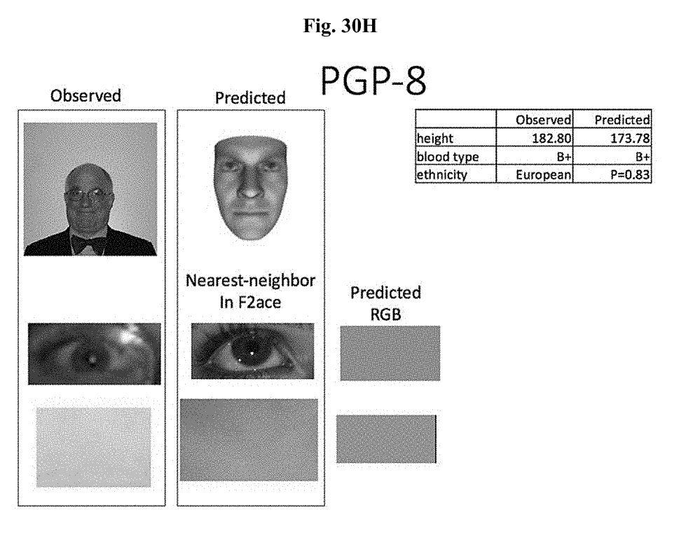

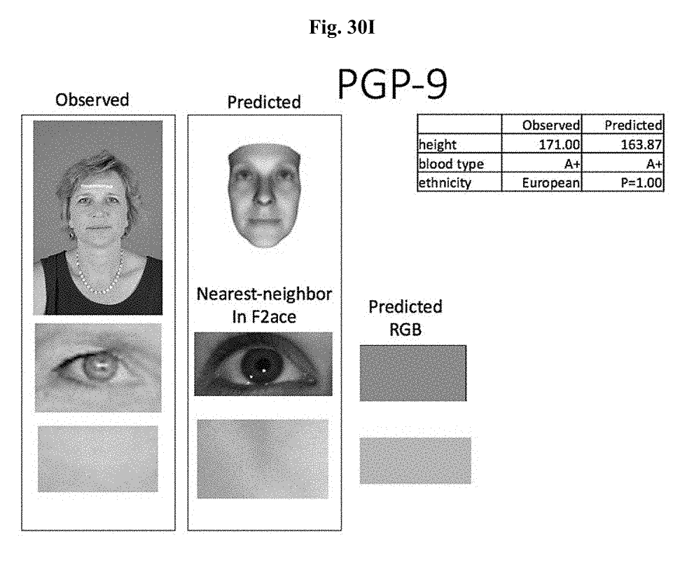

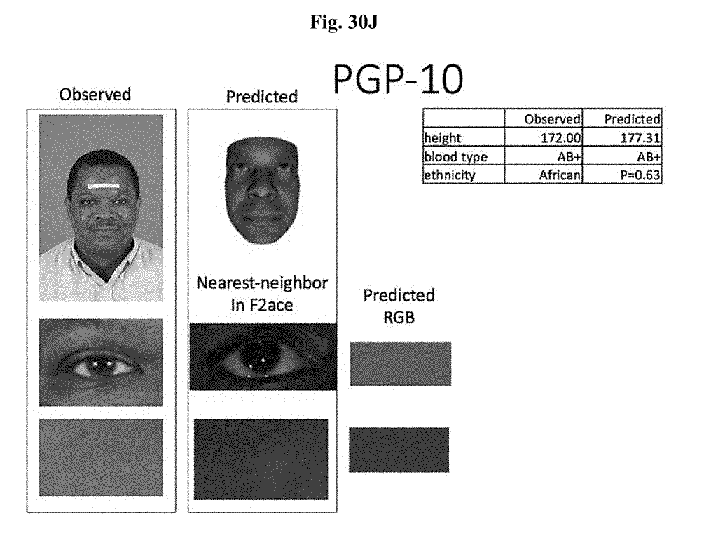

Referring to FIG. 30, in a particular embodiment, an exemplary digital processing device 3801 is programmed or otherwise configured to determine phenotypic traits form a nucleic acid sequence. The device 3801 can regulate various aspects of phenotypic trait determination, facial structure determination, nucleic acid sequence analysis (for both SNPs and PCs), generating graphical representations of faces and audio representations of voice pitch of the present disclosure, such as, for example, ingesting a nucleic acid sequence and rendering a facial structure representation and key phenotypic traits such as height, weight, age, or eye color to a viewing device. In this embodiment, the digital processing device 3801 includes a central processing unit (CPU, also “processor” and “computer processor” herein) 3805, which can be a single core or multi core processor, or a plurality of processors for parallel processing. The digital processing device 3801 also includes memory or memory location 3810 (e.g., random-access memory, read-only memory, flash memory), electronic storage unit 3815 (e.g., hard disk), communication interface 3820 (e.g., network adapter) for communicating with one or more other systems, and peripheral devices 3825, such as cache, other memory, data storage and/or electronic display adapters. The memory 3810, storage unit 3815, interface 3820 and peripheral devices 3825 are in communication with the CPU 3805 through a communication bus (solid lines), such as a motherboard. The storage unit 3815 can be a data storage unit (or data repository) for storing data. The digital processing device 3801 can be operatively coupled to a computer network (“network”) 3830 with the aid of the communication interface 3820. The network 3830 can be the Internet, an internet and/or extranet, or an intranet and/or extranet that is in communication with the Internet. The network 3830 in some cases is a telecommunication and/or data network. The network 3830 can include one or more computer servers, which can enable distributed computing, such as cloud computing. The network 3830, in some cases with the aid of the device 3801, can implement a peer-to-peer network, which may enable devices coupled to the device 3801 to behave as a client or a server.

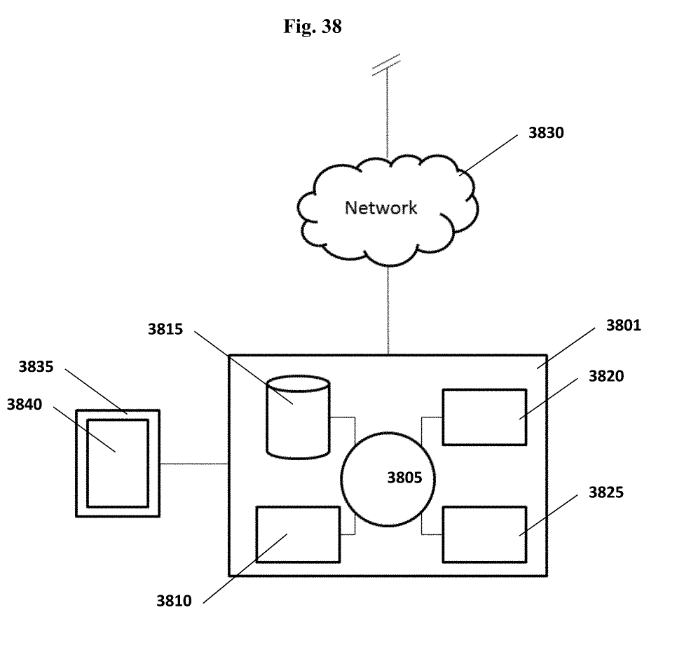

Continuing to refer to FIG. 38, the CPU 3805 can execute a sequence of machine-readable instructions, which can be embodied in a program or software. The instructions may be stored in a memory location, such as the memory 3810. The instructions can be directed to the CPU 3805, which can subsequently program or otherwise configure the CPU 3805 to implement methods of the present disclosure. Examples of operations performed by the CPU 3805 can include fetch, decode, execute, and write back. The CPU 3805 can be part of a circuit, such as an integrated circuit. One or more other components of the device 3801 can be included in the circuit. In some cases, the circuit is an application specific integrated circuit (ASIC) or a field programmable gate array (FPGA).

Continuing to refer to FIG. 38, the storage unit 3815 can store files, such as drivers, libraries and saved programs. The storage unit 3815 can store user data, e.g., user preferences and user programs. The digital processing device 3801 in some cases can include one or more additional data storage units that are external, such as located on a remote server that is in communication through an intranet or the Internet.

Continuing to refer to FIG. 38, the digital processing device 3801 can communicate with one or more remote computer systems through the network 3830. For instance, the device 3801 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Methods as described herein can be implemented by way of machine (e.g., computer processor) executable code stored on an electronic storage location of the digital processing device 3801, such as, for example, on the memory 3810 or electronic storage unit 3815. The machine executable or machine readable code can be provided in the form of software. During use, the code can be executed by the processor 3805. In some cases, the code can be retrieved from the storage unit 3815 and stored on the memory 3810 for ready access by the processor 3805. In some situations, the electronic storage unit 3815 can be precluded, and machine-executable instructions are stored on memory 3810.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

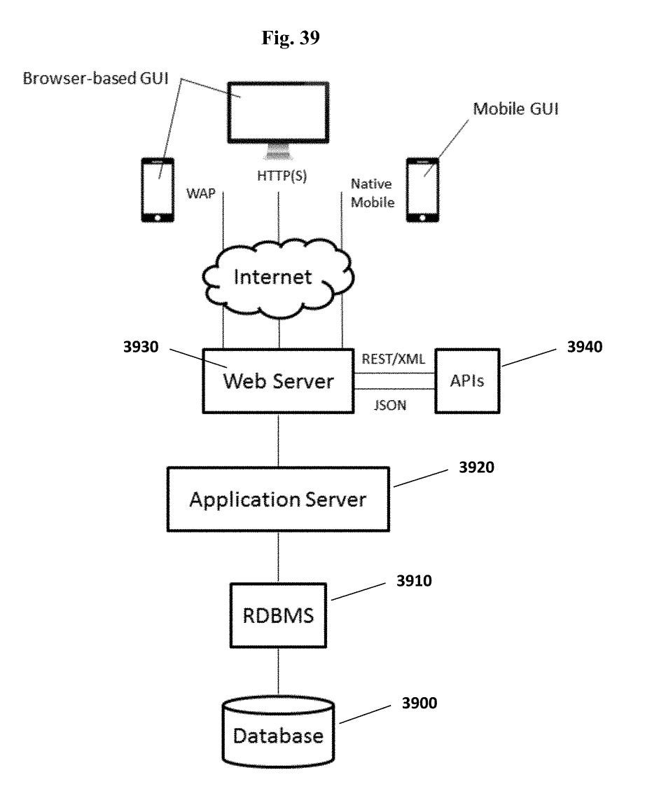

Referring to FIG. 39, in a particular embodiment, an application provision system comprises one or more databases 3900 accessed by a relational database management system (RDBMS) 3910. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 3920 (such as Java servers, .NET servers, PHP servers, and the like) and one or more web servers 3930 (such as Apache, IIS, GWS and the like). The web server(s) optionally expose one or more web services via app application programming interfaces (APIs) 3940. Via a network, such as the Internet, the system provides browser-based and/or mobile native user interfaces.

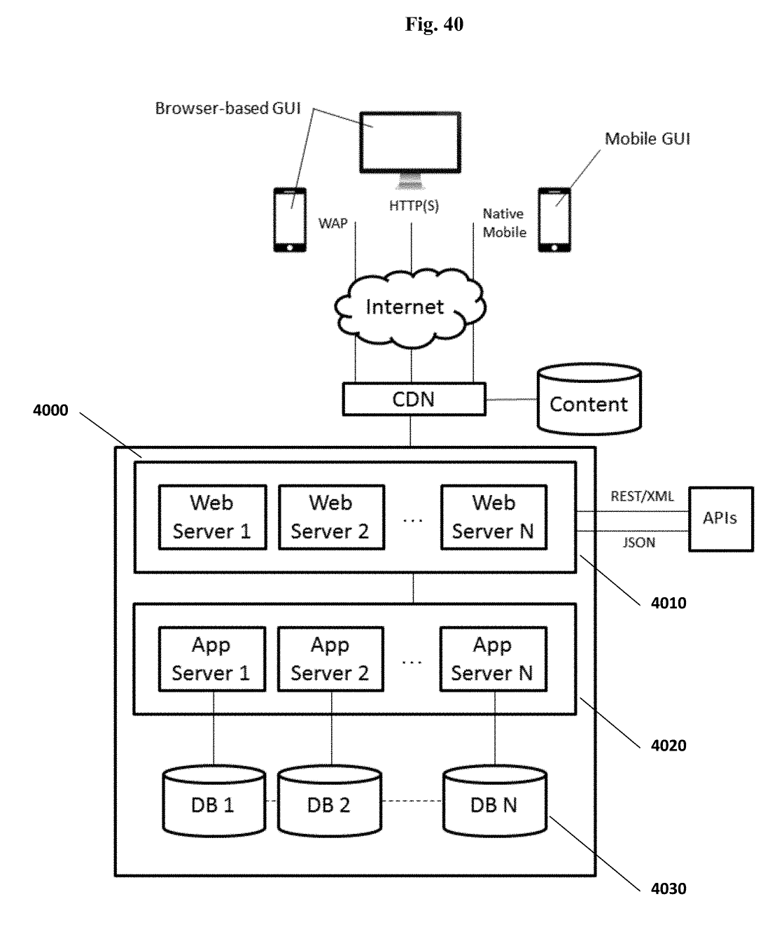

Referring to FIG. 40, in a particular embodiment, an application provision system alternatively has a distributed, cloud-based architecture 4000 and comprises elastically load balanced, auto-scaling web server resources 4010 and application server resources 4020 as well synchronously replicated databases 4030.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities, which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of nucleic acid sequence data and phenotypic traits and measurements. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### EXAMPLES

The following illustrative examples are representative of embodiments of the software applications, systems, and methods described herein and are not meant to be limiting in any way.

### Example 1-Study Overview and Extraction of Phenotypic and Genotypic Data

### Study Population and Methodological Approach

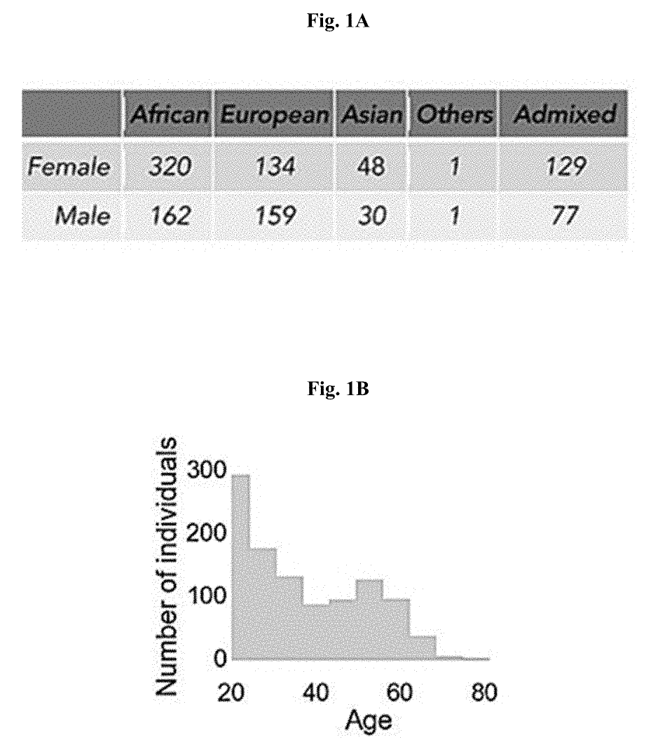

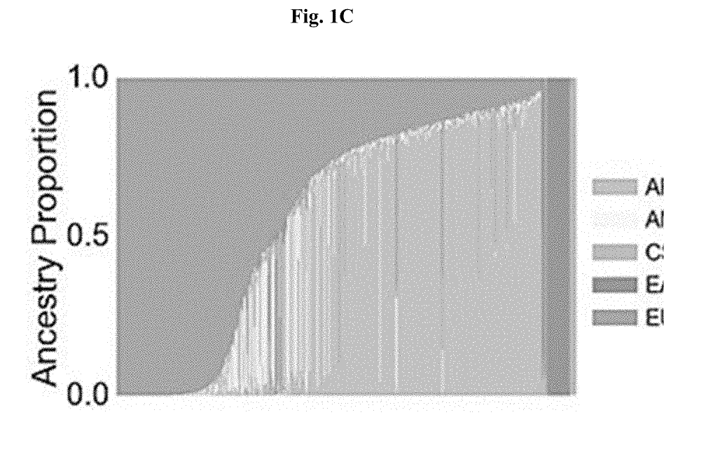

We collected a convenience sample of 1,061 individuals from the San Diego area. Participants for our project were from the greater San Diego area recruited by ads, social media, posting signs on university campuses, and word-of-mouth. Inclusion criteria included both male and female and ≥18 years of age; exclusion criteria included intravenous drug usage, positive for Hepatitis A, Hepatitis B, HIV-1, and/or HIV-2; moustache and/or beard; and pregnant at the time of participation. The resulting study population was ethnically diverse, including 482, 293, 78, and 2 individuals with genomic ancestry inferred to be greater than or equal to 70% from Africa, Europe, Asia, or other regions, respectively. FIGS. 1A and 1C show that the cohort included 206 admixed individuals with less than 70% ancestry from any one group and ancestry proportions inferred from the genome. The age distribution of the study population in FIG. 1B shows that the study also included a diverse representation of ages ranging from 18 to 82 years old, with an average age of 36 years old. Each individual underwent standardized collection of phenotypic data, including high resolution 3D facial images, voice samples, quantitative eye color, quantitative skin color, as well as standard variables such as age, height, and weight.

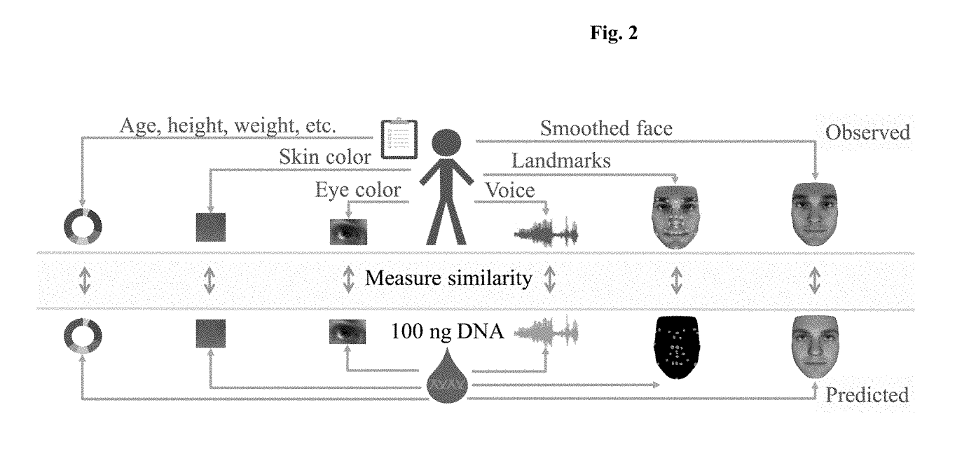

Referring to FIG. 2 the goal was to integrate predictions of each trait in order to measure an overall similarity between the phenotypic profile predicted from the genome and the observed values derived from an individual's image and basic demographic information. We used a strict train-test procedure based on ten-fold cross-validation to produce held-out predictions of each phenotype from the genome. Accuracy for held-out predictions was measured by the fraction of variance in the trait explained by the predictive model (R²_{CV}).

### Collection of Data

Participants self-reported sex, age or date of birth, eye color, ancestry, and approximate hours since last shave. Weight was measured in kilograms (kg) and height in centimeters (cm), both without shoes, using the MedVue Digital Eye-Level Physician Scale with attached height rod (DETECTO Scale Company, Webb City, Mo.).

The face was photographed using the 3dMDtrio System with Acquisition software (3dMD LLC, Atlanta, Ga.); this is a high-resolution three-dimensional (3D) system equipped with 9 machine vision cameras and an industrial-grade synchronized flash system; the 3D 200-degree face was captured in approximately 1.5 milliseconds. If necessary, the participants' hair was pulled away from the face by the use of hairbands and hairpins in order to expose significant facial landmarks. Further, the participants were asked to remove all makeup and facial jewelry, e.g., earrings and nose studs. Each participant sat directly in front of the camera system on a manually controlled height stool; they were asked to focus their eyes on a marking 6″ above the center camera and maintain a neutral expression.

Participants' voices were recorded with both scripted and a 2 minute minimum non-scripted text using the Olympus Digital Voice Recorder WS-822 (Olympus Imaging Corp., Tokyo, Japan) with attached RadioShack Unidirectional Dynamic Microphone (RadioShack, Ft. Worth, Tex.).

We sequenced the full genome of each individual. A minimum of 5 mL EDTA-anticoagulated blood was collected for all 1,061 participants. The blood was stored at room temperature during the day and at the end of each collection session, they were placed in 4° C. storage until extraction. The genome was extracted, quantified, normalized, sheared, clustered, and sequenced. TruSeq Nano DNA HT Library Preparation Kit (Illumina, Inc., San Diego, Calif.) for next generation sequencing library preparation was used following the manufacturer's recommendations. DNA libraries were normalized and clustered using the HiSeq SBS Kit v4 (Illumina, Inc.) and HiSeq PE Cluster Kit v4 cBot (Illumina, Inc.). Sequencing was performed on HiSeq X Ten System sequencers (Illumina, Inc.) using a 150 base paired-end single index read format following the manufacturer's recommendations. We sequenced the full genome of each participant at an average depth of 41×.

### Quantitative Genotyping

We extracted a set of SNPs from 6,299 genome-VCF (Variant Call Format) files of high quality full sequencing samples. These samples comprise a superset of all individuals included in this study. Additional samples were used from other cohorts in our datasets for height estimation. We accepted the calls for the SNPs that passed the standard quality score threshold (PASS variants) of the Isaac variant caller; all other variants were treated as missing. From this initial set of variants, we filtered to a smaller set of SNPs which we used to compute genomic principal components (PCs) by excluding non-autosomal SNPs, SNPs with a minor allele frequency (MAF) f<5%, SNPs with a missing rate ≥10% or larger, or SNPs found to be in Hardy-Weinberg disequilibrium (p≤10⁻⁴) on the 1,061 individuals from our cohort. The final set of variants used consists of 6,147,486 SNPs.

We then constructed the SNP matrix of minor allele dosage values (represented as minor allele counts of 0, 1, or 2). In this matrix, rows represented the individual samples and columns represented the SNPs. Missing variants were imputed to the mean dosage. Each SNP column was scaled by the probability density function (PDF) of a symmetric Beta distribution evaluated at the MAF f.

$$B ( f \vert α ) = \frac{{{f}^{α - 1} ( 1 - f )}^{α - 1}}{{\int }_{0}^{1} {{t}^{α - 1} ( 1 - t )}^{α - 1} dt}$$

We chose a shape parameter α of 0.8 for the symmetric Beta distribution. This yields a U-shaped distribution that up-weights low frequency variants. Such a weighting ensures that low frequency variants have larger effect sizes than common variants. After the imputation and scaling, the genomic PCs were computed from the matrix of dosages of our samples. All 6,299 samples were projected onto the same set of components.

### Landmarking 3D Images and Extracting Landmark Distances

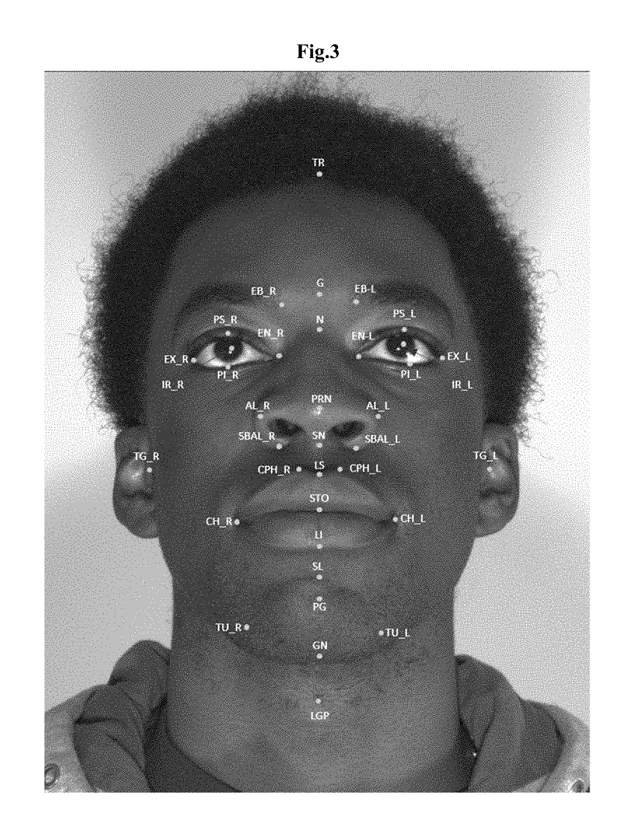

Facial landmarking is an important basic step in our face modeling procedure as they are used to align face images, and to compute landmark distances (e.g., distance between the inner edges of left and right eyes and width of the nose). A total of 36 landmarks for each 3D image was measured using 3dMDvultus™ Software v2.3.02 (3dMD LLC). Each measurement is precise to 750 microns. The landmarks and their definitions were adopted from www.facebase.org (14, 15), with the addition of the laryngeal prominence. The landmarks are shown in Table 1. FIG. 3 illustrates facial landmarks overlaid on an image of a face. The landmarks were placed in order from top, going downward in the center, to the right, then left, and bottom. All landmarks in this study were identified visually, i.e., no palpation; the analyst relied upon the 3dMDvultus Software v2.3.02 to turn the image 360° and applied the Wireframe render mesh of triangles features to annotate each landmark.

TABLE 1
36 landmarks were manually placed on each of the 3D images. The name of the
landmark, the label used in our studies, and the definition of each landmark are
provided. _R and _L signify the same landmark on the right and left side of the face.

| Landmark | Label | Definition |
|---|---|---|
| Trichion | TR | Point where the normal hairline and middle line of |
| | | the forehead intersect. |
| Glabella | G | Mid-point between the eyebrows and above the |
| | | nose; underlying bone, which is slightly depressed, |
| | | and joins the 2 superciliary ridges; cephalometric |
| | | landmark that is just superior to the nasion. |
| Naison | N | Intersection of the frontal bone and 2 nasal bones of |
| | | the human skull; distinctly depressed area directly |
| | | between the eyes, just superior to the bridge of the |
| | | nose; just inferior to the glabella. |
| Eyebrow Right (or | EB_R or L | Lower corner of where the eyebrow begins. |
| Left) | | |
| Endocanthus Right (or | EN_R or L | Innermost corner of eye where tear duct and the skin |
| Left) | | meet. |
| Palpebrale Superious | PS_R or L | Highest point of the upper eyelid, directly above the |
| Right (or Left) | | iris landmark and on the eyelash line. |
| Ectocanthus Right (or | EX_R or L | Outermost corner of the eye. |
| Left) | | |
| Palpebrale Inferious | PI_R o L | Highest point of the lower eyelid, directly below the |
| Right (or Left) | | iris landmark and on the eyelash line. |
| Iris Right (or Left) | Iris_R or L | Center of the pupil. |
| Pronasale | PRN | Tip of the nose. |
| Alar Right (or Left) | AL_R or L | Midpoint of the outer flaring cartilaginous wall of |
| | | the outer side of each nostril. The ala of the nose |
| | | (wing of the nose) is the lateral surface of the |
| | | external nose. |
| Subalar Right (or Left) | SBAL_R or L | Lowest point where the nostril and the skin on the |
| | | face intersect; located inferior to the “alar” |
| | | landmark. |
| Subnasale | SN | Lowest point of the nasal septum intersects with the |
| | | skin of the upper lip. |
| Labiale Superious | LS | Midline, between the philtral ridges, along the |
| | | vermillion border of the upper lip; uppermost point |
| | | in the center of the upper lip where the lip and skin |
| | | intersect. |
| Crista Philtri Right | CPH_R or L | Highest point of the philtral ridges, or crests, that |
| | | intersect with the vermillion border of the upper lip. |
| Chelion | CH_R | Outermost corner, commissure, of the mouth where |
| | | the upper and lower lips meet. |
| Labiale Inferius | LI | Midline along the vermillion border of the lower lip; |
| | | lowermost point in the center of the lower lip where |
| | | the lip and skin intersect; midline along the inferior |
| | | vermillion border of the lower lip. |
| Stomion | STO | Center point where upper and lower lips meet in the |
| | | middle; easily identified when lips are closed, point |
| | | can still be identified when the lips are apart by |
| | | placing the landmark along the inferior free margin |
| | | of the upper lip. |
| Sublabial | SL | Most superior point of the chin, above the pogonion; |
| | | verify with lateral view. |
| Pogonion | PG | Most projecting median point on the anterior surface |
| | | of the chin; verify with lateral view. |
| Gnathion | GN | Inferior surface of the chin/mandible; immediately |
| | | adjacent to the corresponding boney landmark on |
| | | the underlying mandible. |
| Tuberculare Right (or | TU_R or L | The slight depression of the jawline somewhere |
| Left) | | between the gnathion and |
| | | the gonion. |
| Tragion Right (or | TG_R or L | Small superior notch of the tragus (cartilaginous |
| Left) | | projection just anterior to the auditory meatus). |

The landmark annotations were carefully determined; some of the landmark positions required careful examination at different angles. For example, pronasale is the most protrusive point on the tip of nose; the image must be turned 90° to accurately place this landmark. Given the annotated landmarks, we defined 27 facial landmark distances between a pair of landmarks, shown below in Table 2.

TABLE 2
Calculated facial landmark distances. Of the 36 landmarks in Table 1,
distances could be calculated from any 2 landmarks; using 3
landmarks, a polyarc (pa) curved line distance across 3 landmarks
was calculated. Below is a partial list of the distances measured.

| Landmark | |
|---|---|
| distance symbol | Definition |
| TGL_TGRpa | Measurement from TG_R through the PRN to TG_L; |
| | it is actually a diarc with two arcs combined; curved |
| | line from left ear to right ear through the pronasale. |
| TR_GNpa | Measurement from TR through the PRN to the GN; |
| | diarc of two arc measurements combined; curved line |
| | from the hairline to just underneath the chin through |
| | the pronasale. |
| EXR_ENR | Width of the right eye |
| PSR_PIR | Height of the right eye |
| ENR_ENL | Distance from inner left eye to inner right eye |
| EXL_ENL | Width of the left eye |
| EXR_EXL | Distance from outer left eye to outer right eye |
| PSL_PIL | Height of the left eye |
| ALL_ALR | Width of the nose |
| N_SN | Height of the nose |
| N_LS | Distance from top of the nose to top of upper lip |
| N_ST | Distance from top of the nose to center point |
| | between lips |
| TGL_TGR | Straight distance from left ear to right ear |
| EBR_EBL | Distance from inner right eyebrow to inner |
| | left eyebrow |
| IRR_IRL | Distance from right iris to left iris |
| SBALL_SBALR | Width of the bottom of the nose |
| PRN_IRR | Distance from the tip of the nose to right iris |
| PRN_IRL | Distance from the tip of the nose to left iris |
| CPHR_CPHL | Distance separating the crests of the upper lip |
| CHR_CHL | Width of the mouth |
| LS_LI | Height of lips |
| LS_ST | Height of upper lip |
| LI_ST | Height of lower lip |
| TR_G | Height of forehead |
| SN_LS | Distance from bottom of the nose to top of upper lip |
| LI_PG | Distance from bottom of the lower lip to the chin |

### Extracting Facial Embedding

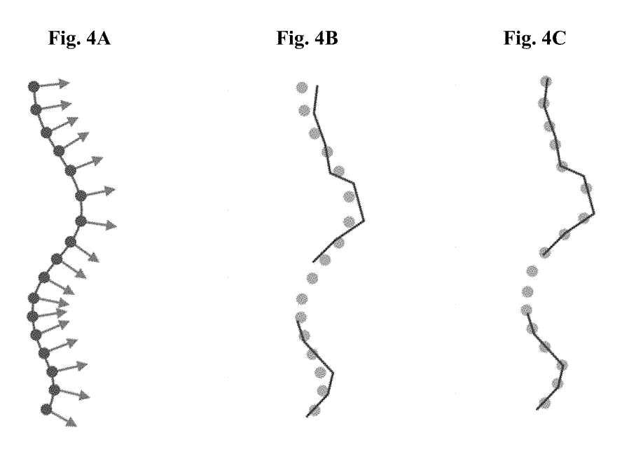

To predict facial structure from genome effectively, we used a low dimensional numerical representation of face, which adequately represents intra-individual variation. For this purpose various algorithms have been used including principal component analysis (PCA), linear discriminant analysis, neural networks, and others. In this disclosure, we used PCA because it allows us to discriminate different faces, and importantly, to reconstruct predicted faces. We start from a neutral 3D face template and align this template in a non-rigid manner to the 3D scans using an expectation maximization (EM) algorithm. At each iteration we approximate correspondences between the 3D scan and the deformed version of the template mesh (E step) and optimize deformation parameters to bring the established correspondences as close to each other as possible (M step). Because the deformation is a global operation and it applies to the entire face image, the set of correspondences might change after the M step. Iteration was performed until the error (i.e., distance between the template face and the 3D scan) is minimized. The deformation model is 3D thin plate splines where the degrees of freedom are the weights of knots manually placed on the template mesh. Once the template model was deformed to match the 3D scan, we computed a displacement over the template mesh to capture the fine scale surface details in our 3D scans. Specifically, rays were traced along the normal vectors of the template mesh and displaced template vertices to the intersection points of these rays with the 3D scans, as illustrated in FIGS. 4A-C. To minimize the noise due to face image misalignment between different face samples, 3D face images were aligned by matching the vertex of the average template face and each individual face. FIG. 4A shows the vertices of the average template face and their normal vectors. In FIG. 4B gray vertices represent the vertex in the average template. Red solid lines represent the scanned face surface for the observed samples. FIG. 4C shows that average face template vertices are displaced along their normal vectors to the closest observed scanned surface. If there is no scanned surface near the template vertices, the closest scanned surfaces are estimated using a Poisson method. This also allowed us to copy the colors from 3D scans onto the template mesh. The areas on the template mesh where the rays do not intersect the scan (either due to noise or scanning problems) were filled using Poisson image editing Using these procedures, a deformed template mesh was obtained and aligned to every 3D scan. Because the purpose of facial embedding is not to capture variations in position and orientation of the head at the time of the scan, we aligned the deformed version of the template to the original template mesh. This final alignment was performed using a rigid body transform.

The observed color of the face is a product of the skin reflectivity and the incident lighting from the environment. Skin reflectivity is a measurement we attempted to phenotype; however, we did not have the precise measurement of incident illumination. Thus, we created a first order approximation by assuming that skin reflectivity is diffuse (incident light at a point is scattered equally in all outgoing directions) which is approximated by albedo, or a reflection coefficient. Albedo, which models faces under different lighting conditions, yields a bilinear form, and was solved by iterating the following steps alternatively until convergence: (1) estimate albedo while keeping incident lighting fixed; (2) estimate incident lighting, which was assumed to be constant across the face images while keeping the albedo constant. Finally, we obtained our face embedding that consists of PCs from all vertex positions on the deformed template, and the solved surface albedo at every vertex.

### Extracting Eye Color

To extract eye color, we used the 2D face images. Then, we employed a LeNet convolutional neural network (CNN) to locate eyes in facial images and extracted the left and right eyes. We manually extracted eye locations for the images where the CNN failed. An example of an extracted eye position is shown in FIGS. 5 and 5B. FIG. 5A shows an eye image extracted from a face image. FIG. 5B shows the identified iris by the blue shaded area.

We performed the following procedure to extract iris pixels: (1) converted each eye image to gray scale and performed OpenCV histogram normalization to improve the contrast of the image; detected edges using a radial edge detector based on the Sobel operator and chose the iris circle by finding the locations that best match the detected edge signal; located the convex hull of the iris circle; eliminated the pupil area by blocking a fixed radius of size around the center of the circle; and; calculated the brightness histogram for the points in the iris circle, and retained the points in the middle 80% of the histogram, which eliminated reflections and any remaining black pupil points. The result is a set of identified iris pixels. We represent these pixels in the RGB color space and calculate the mean value for each R, G, and B parameter to obtain an overall iris color for the eye. We found that the measured eye color for the 2 eyes was very close, thus, we used an average of both eyes as the raw color values for the subject.

### Extracting Skin Color

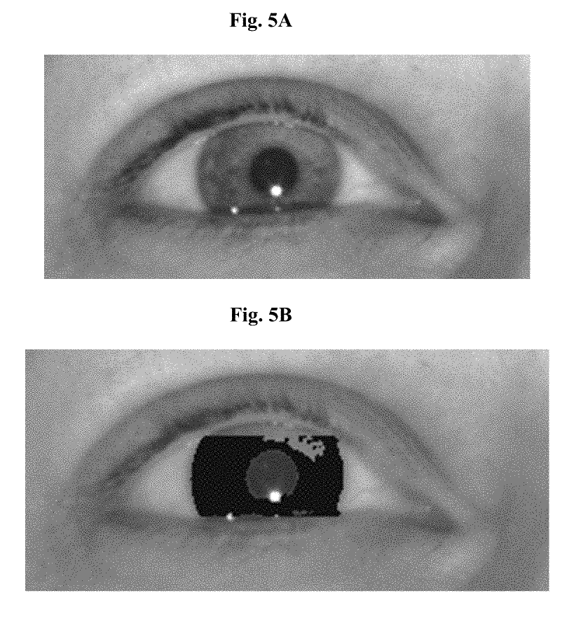

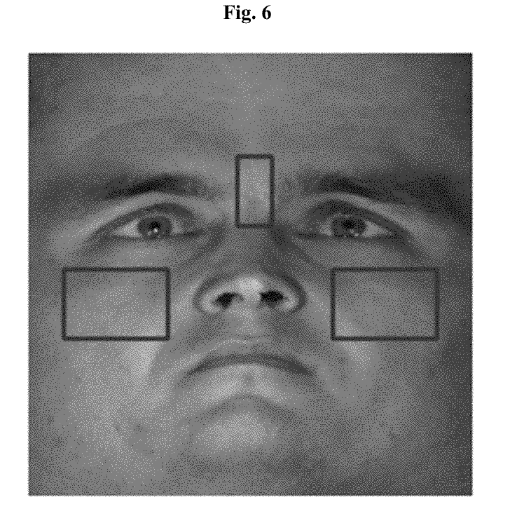

To obtain skin color from the 2D image scan, we extracted 3 skin patches (one patch from the forehead and 2 from the cheek just below each eye) from albedo-normalized and aligned face photos as shown in FIG. 6. To remove the outliers in the skin color, we used K-medoid clustering (k=3), and chose the RGB values for the cluster center with the medium lightness to account for non-uniform light reflection from the skin surface.

### Extracting Voice Embedding

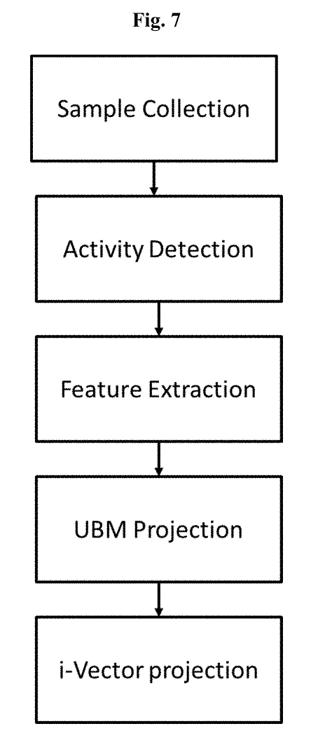

The Spear open-source speaker recognition toolkit was used to create low-dimensional voice feature vectors. See E. Khoury, L. El Shafey, S. Marcel, in ICASSP, IEEE International Conference on Acoustics, Speech and Signal Processing—Proceedings (2014), pp. 1655-1659 These vectors are referred to as identify-vectors or i-vectors, obtained by a joint factor analysis as shown in FIG. 7. The spear toolbox transforms voice samples into i-vectors through a multi-step pipeline process. After a voice sample is collected, it uses an activity detector based on audio energy to trim out silence from the sample. Next, the Spear toolbox applies a Mel-Frequency Cepstrum Coefficient feature extractor that converts successive windows of the sample to Mel-Frequency Cepstrums. Finally, it projects out the UBM to account for speaker- and channel-independent effects in the sample, and computes the i-vector corresponding to the original sample.

### Ridge Regression for Trait Prediction

We evaluated all models using a modified 10-fold cross-validation (CV), where samples were placed in each set based on a hash function on an anonymized subject identifier. This process is equivalent to uniform sampling of a fold, and the expected number of samples per fold is the same per fold.

For each of ten repetitions we used nine folds as a training set, and the remaining fold as the test set so that each individual was predicted out-of-sample exactly once. When computing the test error on one fold, we chose a tuning parameter for ridge regression using five-fold CV within the training set. CV folds were identical across different models and were chosen to avoid splitting related individuals between training and test sets (e.g., siblings were included in either training set or test set, rather than both). This decision was made to prevent correlation between train and test sets, since closely related relatives share not only a large portion of the genome but environmental factors which can cause over-fitting.

Unless stated otherwise, we fit a ridge regression on the training data set, where the regularized sum of squares was minimized over an offset c and a set of regression coefficients β_{d}. For a given individual with index n out of Nₜᵣₐᵢₙ training samples, the residual rₙ is defined as the difference between the phenotype value yₙ and a linear regression in the covariates x_{nd}:

rₙ=yₙ−(c+Σ_{d=1}^{D}x_{nd}β_{d}).

The optimal coefficients are given by

argmin_{β,c}Σ_{d=1}^{N}^{ₜᵣₐᵢₙ}rₙ²+α(Σ_{d=1}^{D}β_{d}²).

For each repetition, an optimal regularization parameter α was estimated by a standard nested five-fold CV over the training data. Given α, we predicted the phenotype on the remaining set of test individuals.
We measured prediction accuracy using the out of sample measure

$${R}_{cv}^{2} = 1 - \frac{{\sum }_{n = 1}^{{N}_{test}} {r}_{n}^{2}}{{\sum }_{n = 1}^{{N}_{test}} {( {y}_{n} - \overline{{y}_{test}} )}^{2}} ,$$

where y̅ₜ̅ₑ̅ₛ̅ₜ̅ is the mean of the test data. This measure has a negative expectation for random predictions. Also, because the model has been fit to the training data set, it is not expected to improve by adding more covariates to the model.

### Example 2-Predicting Sex from a Biologic Sample

To predict sex from the genome, we first estimated the copy number for chromosome X (CCN_chrX) and Y (CCN_chrY). Males are expected to have one copy of chromosome X and one copy of chromosome Y and females are expected to have two copies of chromosome X. FIG. 8 shows the distributions for CCN_chrX vs CCN_chrY computed for all the samples in our dataset. Sex can be predicted by inspecting the plot in FIG. 8. We made rule-based sex prediction as follows: samples with CCN_chrY≤0.25 were predicted as female, regardless of the value of CCN_chrX. Samples with CCN_chrY>0.25 were predicted as male. Among male samples in our dataset, we observed a case with XXY aneuploidy, also known as Klinefelter's syndrome. This case was identified with the following rule: 1.5<CCN_chrX≤2.5. We can easily extend these rules to address other cases of sex chromosome aneuploidy, if necessary. When compared to manual sex annotations, our chromosome copy number (CCN)-based rules achieved an accuracy of 99.6%. Four inconsistencies and two missing annotations were observed in 1,061 samples. For the four errors, three female samples were predicted as male and one male sample was predicted as female. A closer look at these cases indicated that all of them were in fact annotation errors. The sample with Klinefelter's syndrome, karyotype 47, XXY, was annotated and predicted as male, as expected. Our sex prediction from CCN of the genome is highly accurate and could be used to identify errors in manual sex calls.

Computing Chromosome Copy Number Variation from WGS Data

We used chromosomal copy number (CCN) for sex determination and to quantify the mosaic loss of sex chromosome. See example 3 predicting age from a biologic sample. Naturally, read depth (RD) at a chromosome could be used to compute the CCN. However, a large proportion of ChrY is paralogous to some autosomal regions. Many of the reads that mapped to ChrY originate from autosomes. For this reason, prior to computing the copy number of ChrY, we filtered the reads to those that mapped uniquely to ChrY. More generally, given the HG38 reference genome (RG), we produced a set of uniquely mappable regions, i.e., regions where any 150-mer can be mapped only once throughout the RG. We first simulated 150 bp-long reads from the RG at each base position of the genome, and then mapped them to the RG using BWA-mem. Next, we collected the source regions from where the reads originated and mapped only once. Lastly, we removed some repetitive regions annotated by RepeatMasker as “low_complexity”, “retroposon”, “satellite” and “SINE” due to lower region coverage as these regions are more difficult to align. We then selected uniquely mappable regions with length >5 kb. The length threshold was determined so that each chromosome contained at least 200 bp of each region. GC bias is known to affect coverage substantially. We computed RD of each region using samtools mpileup command, and grouped the regions by GC content. For a particular GC content group, the median value of the RD at autosomal regions was used as the baseline value denoted as rdgc. Here, we assumed a healthy person to have a diploid genome and no detectable mosaic loss of autosomes. For a region in this GC group, CCN was computed as twice the observed RD divided by rdgc. For a given chromosome c, the CCN was computed as the median CCN of all the regions contained within c.

### Example 3 Predicting Age from a Biologic Sample

Age is a critical phenotypic trait for forensic identification. Accurate genomic prediction of age is especially important in our context, as age was used as a covariate for the prediction of other phenotypes. The maximum depth of the tree and the minimum number of samples per leaf were tuned by cross-validation within each training fold. Since we aim to evaluate this model for forensic casework using only genomic information, we substituted genome predicted age for actual age in every applicable phenotype model. To predict age from the genome, we fit a random forest regression model that used a person's average telomere length estimate and estimates of chromosome X and Y copy numbers as covariates for predicting age. During training, we removed samples that were considered outliers. For our purposes, an outlier was defined as any male sample with an estimated Y copy number below 0.95 or above 1.05, or any female sample with an estimated X copy number below 1.95 or above 2.05.

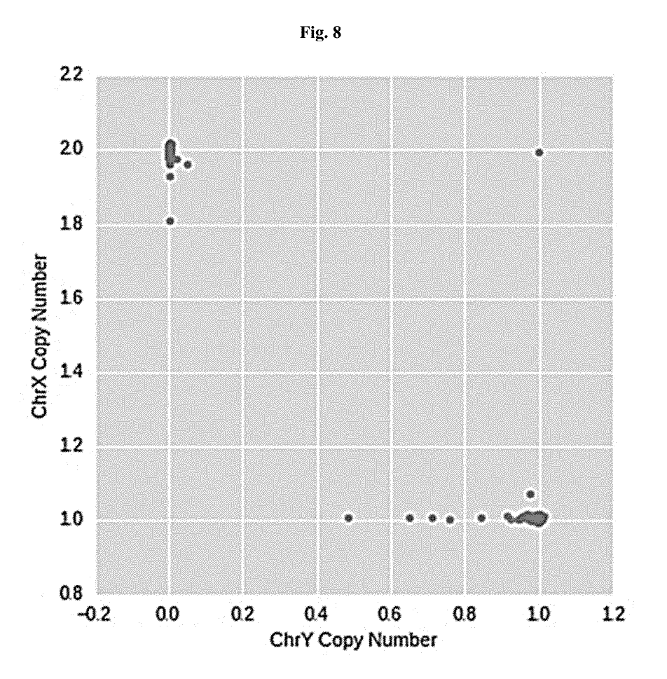

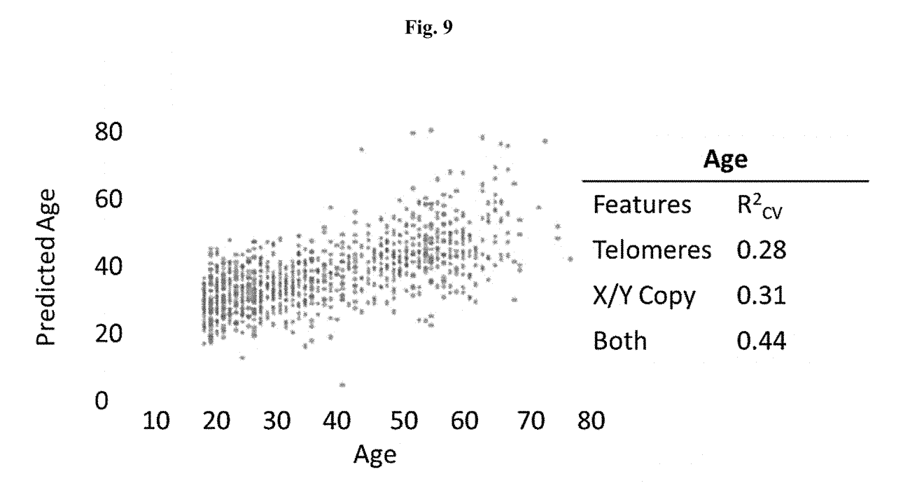

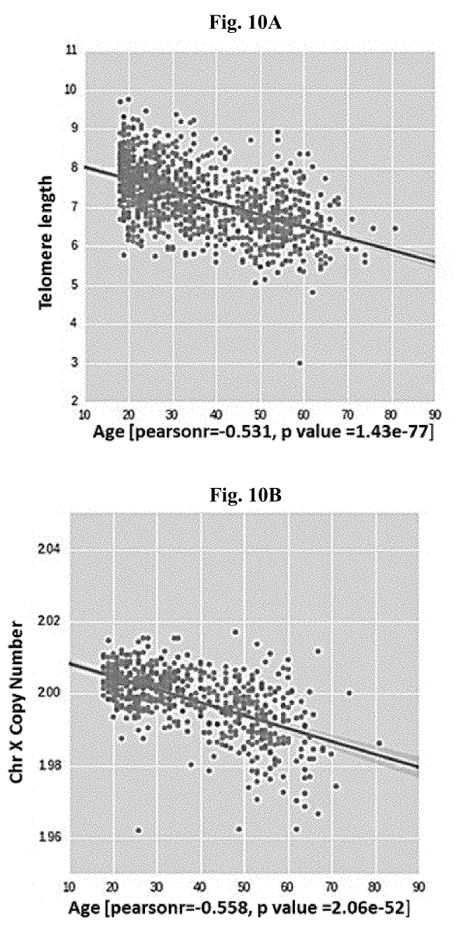

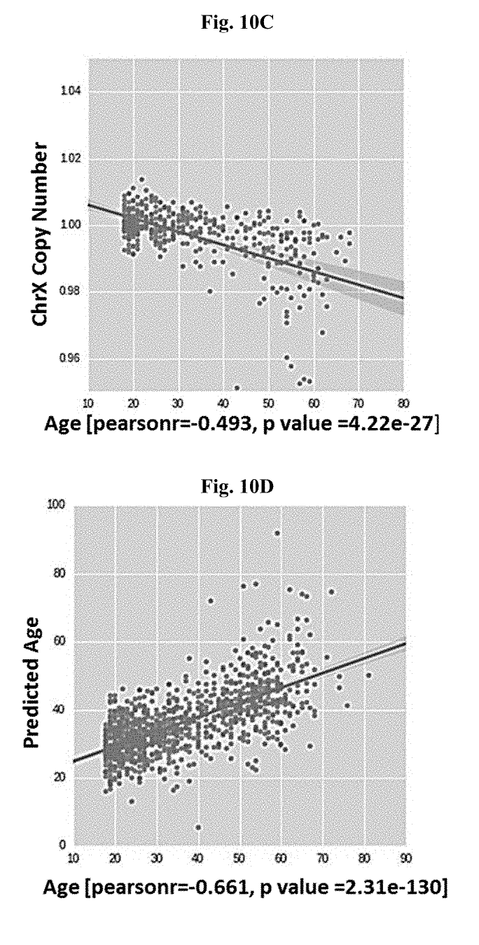

Reduction in telomere length can be estimated from next-generation sequence data based on the proportion of reads that contain telomere repeats. Here, we were able to predict age from telomere length with R²_{CV}=0.28 as shown in FIG. 9. Previously, telomere length from whole genome sequence data has been used to predict age with an R² of 0.05. One key to our comparatively high level of accuracy was the use of repeatedly sequenced samples to choose the repeat threshold for classifying reads as telomeric. Another important factor is the high reproducibility and even coverage of the genome In addition to telomere length estimates, we detected mosaic loss of the X chromosome with age in women from whole genome sequence data. In men, no such effect has been observed, presumably because at least one functioning copy of the X chromosome is required per cell. However, we were able to use whole genome sequence data to estimate mosaic loss of the Y chromosome with age in men. Mosaic loss of sex chromosomes was computed from chromosome copy number variation as previously explained. Together, as shown in FIG. 9, telomere shortening and sex chromosome loss were predictive of age with an R²_{CV} of 0.46 (mean absolute error (MAE)=8.0 years). FIGS. 10A-10C show the regression plots of telomere length estimates (14), and chromosomal copy number for chromosomes X or Y (chr[X|Y] CCN) versus age. FIG. 10D shows the predicted versus expected age for all our samples using both telomere length and sex chromosome mosaicism. Specific somatic DNA rearrangements, called single joint T-cell receptor excision circles (sjTRECs) in T lymphocytes can be correlated with age. Therefore, we investigated whether sequences from the sjTRECs could be reliably detected in our genome sequencing data and used as a marker for age. In our investigation, sjTRECs did not show significant signal for age discrimination and we did not use it for our age prediction model. Instead, this particular marker worked well in qPCR assays perhaps due to the amplification step that exponentially increased the abundance of non-replicated circular sjTRECs which are serially diluted with each cellular division. Thus, the methods of this disclosure can be augmented by using existing assays based on qPCR on a specific sjTREC such as δRec-ψJα.

Estimating Telomere Length from WGS Data

We estimated the telomere length from WGS data as the product of the size of human genome and the putative proportion of the telomeric read counts out of total read counts. We considered a read to be telomeric if it contained k or more telomere patterns (CCCTAA or its complement), where k is the telomere enrichment level. Thus, the estimated telomere length of sample x, denoted as tₖ(x) was computed as:

$${t}_{k} ( x ) = \frac{M ( x ) {r}_{k} ( x ) S}{R ( x ) N}$$

where M(x) is a calibration factor for x which controls for systemic sequencing biases introduced by the reagent chemistry (DNA degradation and other sources), rₖ(x) is the count of putative telomeric reads obtained for telomere enrichment level k, S is the size of the human genome (gaps included), R(x) is the sample's total read count, and N is fixed at 46 for human, the number of telomeres in the genome. To identify an optimal telomere enrichment level k, we performed measurement error analysis on 512 WGS runs of the reference sample NA12878. These 512 WGS runs used the same reagent chemistry and were made around the same dates as our cross-validation dataset. We estimated telomere lengths with above formula for all runs and enrichment levels. For the measurement error analysis we compare Repeatability (R) between different values of k. Repeatability (R) was estimated as the variance derived from genetic and environmental effects divided by the total phenotypic variance, or R=1−v₁/vₚ, where vₚ is the telomere length variance over our cross-validation dataset and v₁ is the length variance computed on NA12878 samples only. In general, repeatability can also be interpreted as the proportion of total variance attributable to among-individual variation. We considered the most repeatable of these runs as our best solution based on the assumption that the true telomere length was constant across all the runs. We produced repeatability index curves versus k over all NA12878 samples. We found that the curve reached its maximum value of 0.752, for k=4. This means that from all the possible values of k, the smallest variance across all sequencing runs of NA12878 for our telomere estimate was 4. We also produced the Pearson correlation coefficient between telomere length estimates and annotated age for our cross-validation set and for all values as shown in FIG. 10A. The best correlation was also obtained at k=4, validating the choice of k based on repeatability. We set the constant factor M(x) such that the distribution of tₖ(x) had a mean value of 7.0, which roughly matched the average reported telomere length obtained through experimental methods using mean terminal restriction fragment (mTRF). For the chemistry used in our dataset, M(x) was equal to 1.0.

### Extracting Single Joint T-Cell Receptor Excision Circles

We extracted specific structural signatures derived from the somatic excision events at the δRec-ψJα site. Specifically, we identified the reads that aligned across the junction of the circular sjTREC, as well as the reads that aligned across the junction of the site of deletion. These junction reads were mapped to 2 genomic locations on chr14 at a distance of ˜88 Kb apart. For better sensitivity, the junction reads included both “split reads” as well as the “discordant read pairs” with 2 paired ends mapped to the 2 distinct locations of interest. The number of junction reads ranged from 0 to 3 across the samples that we selected from different age groups. Due to the relatively weak signal that we observed in these selected samples, the sjTREC signatures identified from our whole genome sequencing did not provide sufficient discriminative power for age prediction.

### Example 4-Predicting Height, Weight, and BMI from a Biologic Sample

To predict the height, weight, and BMI of each individual, we built on previously reported polygenic predictors, applied a study-specific adjustment to the set of reported effect sizes, and added genomic PCs to the model. As shown in Table 3, we calculated strong performance for the prediction of height (R²_{CV}=0.59, MAE=5 cm); and weaker performance for the prediction of weight (R²_{CV}=0.20, MAE=14.4 kg), and BMI

$${{R}^{2}}_{CV} = 0.06 , MAE = 4.8 \frac{kg}{{m}^{2}} .$$

TABLE 3

| | Height | Weight | BMI |
|---|---|---|---|
| Features | (R²cv) | (R²cv) | (R²cv) |
| Age | 0.05 | 0.01 | 0.00 |
| Age + sex | 0.52 | 0.15 | 0.00 |
| Age + Sex + Genomic | 0.55 | 0.21 | 0.06 |
| PCs | | | |
| Age + Sex + Genomic | 0.59 | 0.21 | 0.12 |
| PCs + SNPs | | | |

To build the height, BMI, and weight genomic predictor, we included 4,082 individuals from 7 different studies in the model building procedure after filtering individuals <18 years old. We included age, sex, the first 100 genomic PCs, and associated SNPs from other studies in our height prediction model. We used 696 SNPs previously identified as height-associated SNPs from large-scale GWAS meta-analysis for the height prediction model (we excluded one SNP rs2735469 among 697 previously identified SNPs since it did not pass our MAF threshold of 0.1% in our data set). For the BMI prediction model, we included 96 SNPs from previously identified as BMI-associated SNPs (we excluded 1 SNP rs12016871 among the reported SNPs because its MAF<0.1%). For the weight prediction model, we used both the height-associated 696 SNPs and BMI-associated 96 SNPs. We used self-reported age and predicted sex from the genome as covariates. We computed the first 100 genomic PCs from our study cohort, and then computed the first 100 PCs for an additional 3,000 individuals in our database by projecting their genomes into the PC space.

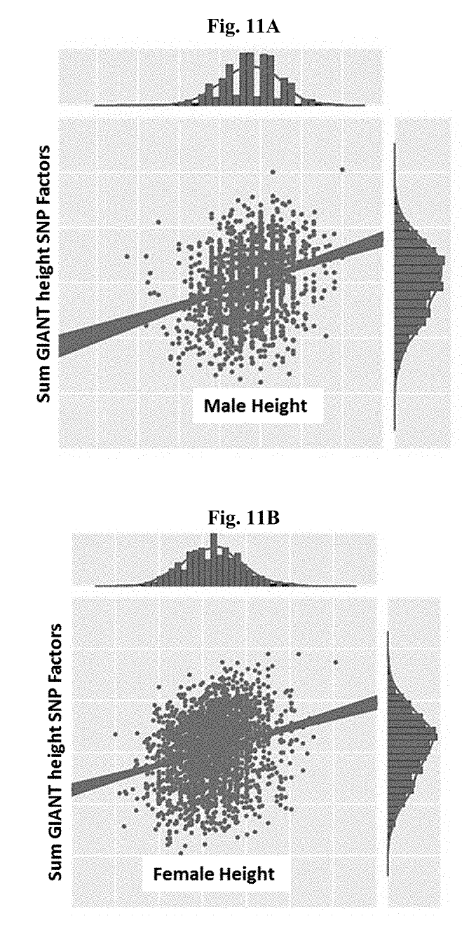

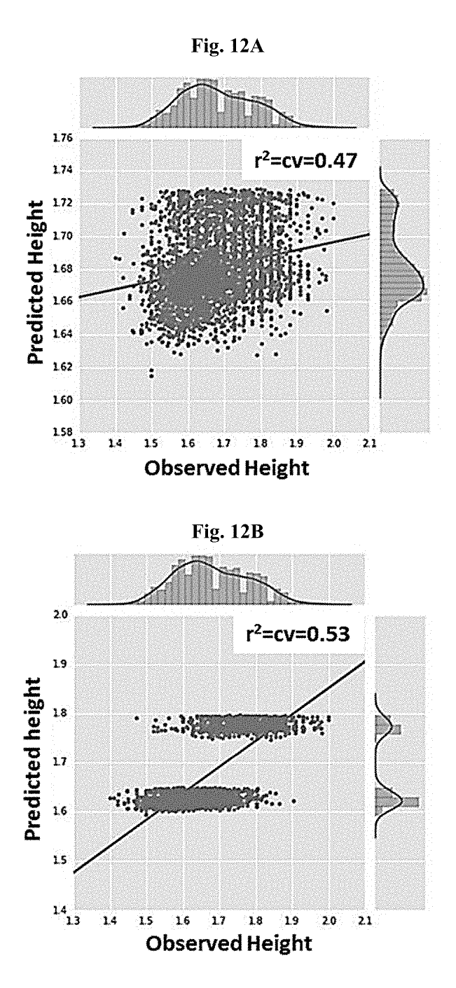

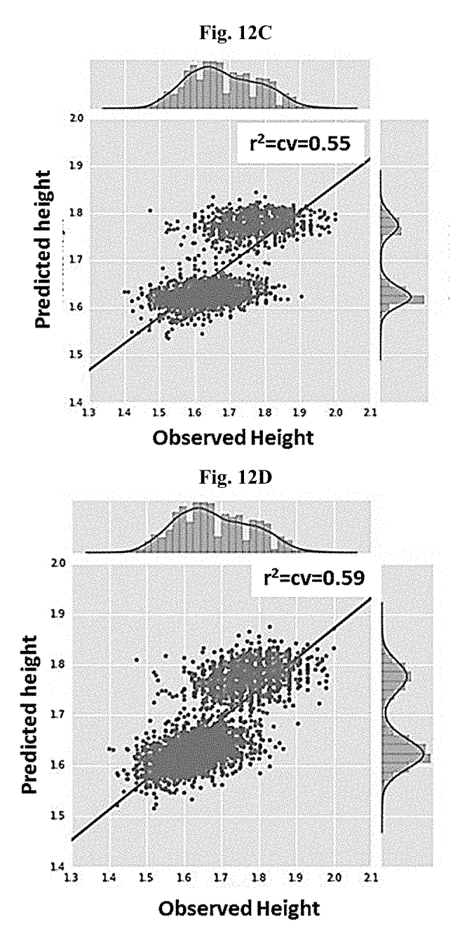

The true effect size of each of the selected SNPs for height/BMI/weight was expected to be small and it would be difficult to accurately estimate these effect sizes on our cohort. Thus, instead of estimating the effect size of 696 SNPs+96 SNPs on samples from our database, we used the previously estimated effect sizes from a large scale meta-analysis of 253,288 individuals of the GIANT consortium for height SNPs; See A. R. Wood et al., Defining the role of common variation in the genomic and biological architecture of adult human height. Nat. Genet. 46, 1173-1186 (2014); and 339,224 individuals for BMI SNPs. See A. E. Locke et al., Genetic studies of body mass index yield new insights for obesity biology. Nature. 518, 197-206 (2015). Then, for height and BMI predictions, one aggregated feature was created for height and BMI which is the sum of 696 SNPs and 96 SNPs weighted by their effect sizes, respectively. FIGS. 11A and 11B shows the relationship between the weighted sum of the GIANT SNP factors and observed male and female height. Table 4 and FIGS. 12A-12D show the mean absolute error (MAE) and R, between the observed and predicted heights by our model with different features.

TABLE 4
Prediction quality for different covariates in ridge regression.
The best predictive model includes age, sex, top 100 genomic
PCs and 696 height associated SNPs in the model.

| Covaiiates | R_{cv}² |
|---|---|
| Mean | −0.001 |
| Mean + age | 0.047 |
| Mean + age + sex | 0.535 |
| Mean + age + sex + 100 PCs | 0.555 |
| Mean + age + sex + 100 PCs + 696 SNP Height | 0.595 |

The prediction model including only age as a feature in FIG. 12A has an MAE of 8.18 cm and R²_{cv} of 0.047. The prediction model with age and sex as in FIG. 12B has an MAE of 5.52 cm and R²_{cv} of 0.535. The prediction model with age, sex and the first 100 genomic PCs as in FIG. 12C has an MAE of 5.30 cm and R²_{cv} of 0.555. When we also included 696 height-associated SNPs into the previous model, we achieved the best predictive model. The prediction model with age, sex, the first 100 genomic PCs, and the 696 height-associated SNPs as in FIG. 12D has an MAE of 5.00 cm and R²_{cv} of 0.595.

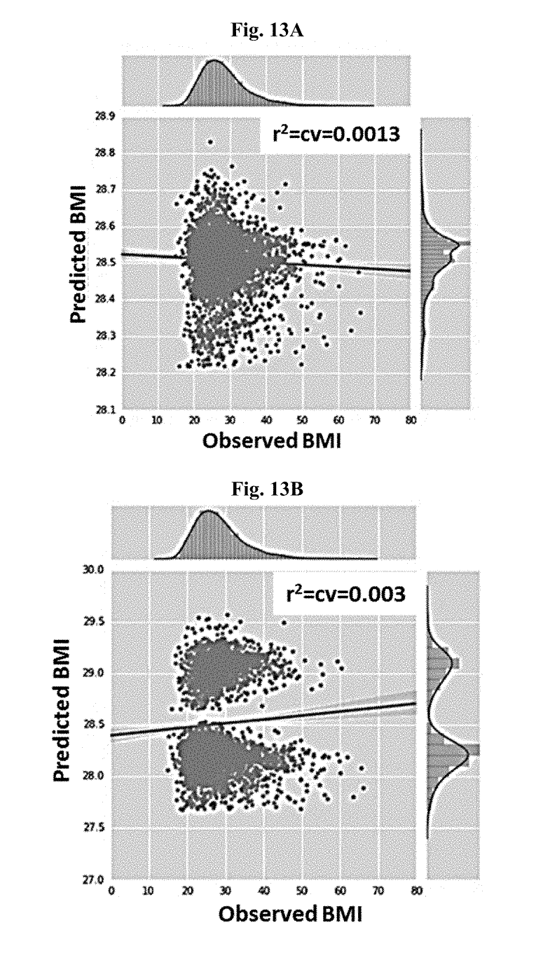

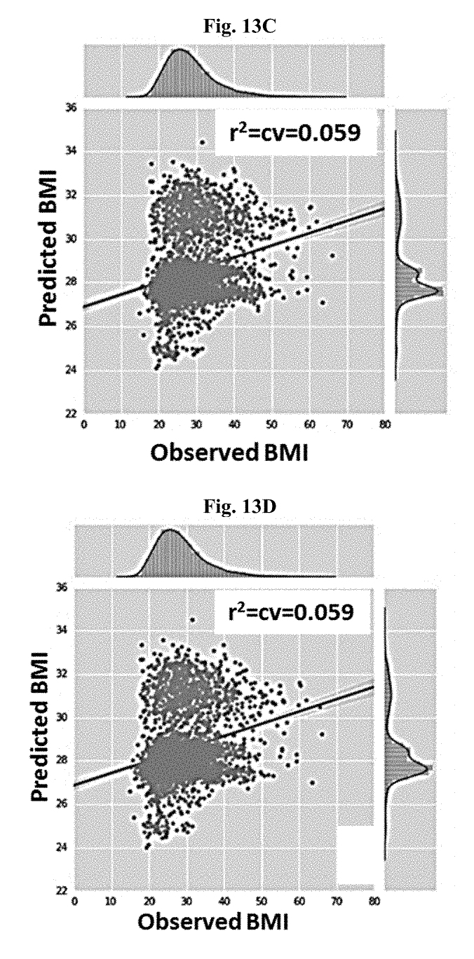

Table 5 shows the MAE and R²_{cv} between the observed and predicted BMI by our model with different features. When the BMI predictive model includes only age as a feature as in FIG. 13A, the MAE is 5.008 kg/m² and R²_{cv} of ˜0.001. The prediction model with age and sex as in FIG. 13B has an MAE of 4.984 kg/m² and R²_{cv} of 0.003. The prediction model with age, sex and first 100 genomic PCs as in FIG. 13C has an MAE of 4.845 kg/m² and R²_{cv} of 0.059. When we also add 96 BMI associated SNPs to the above model, we achieved the best predictive model in terms of MAE. The prediction model with age, sex, the first 100 genomic PCs, and 96 BMI-associated SNPs as in FIG. 13D has an MAE of 4.843 kg/m² and R²_{cv} of 0.059.

TABLE 5
Prediction quality for BMI with different covariates in ridge
regression. The best predictive model includes age, sex, top
100 genomic PCs and 96 BMI associated SNPs in the model,

| Covatiates | R_{cv}² |
|---|---|
| Mean | −0.001 |
| Mean + age | −0.001 |
| Mean + age + sex | 0.003 |
| Mean + age + sex + 100 PCs | 0.059 |
| Mean + age + sex + 100 PCs + 96 SNP WI | 0.059 |

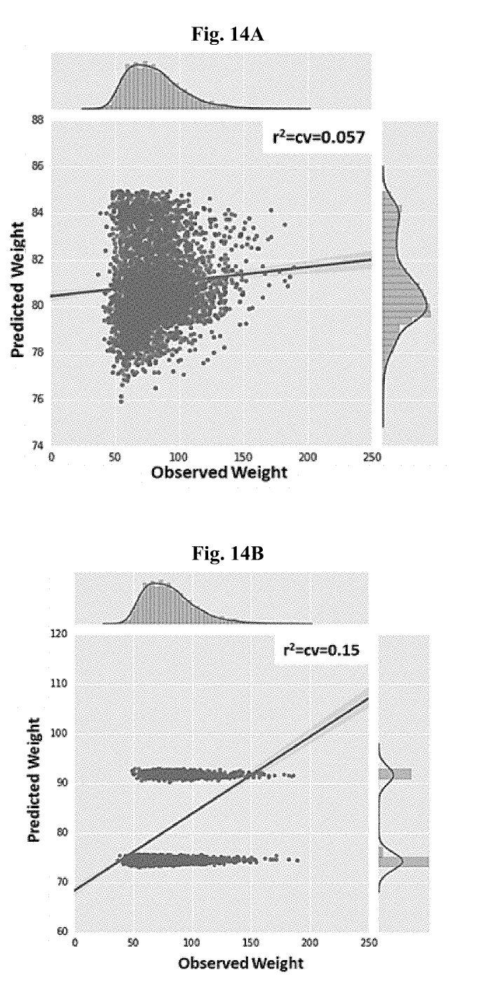

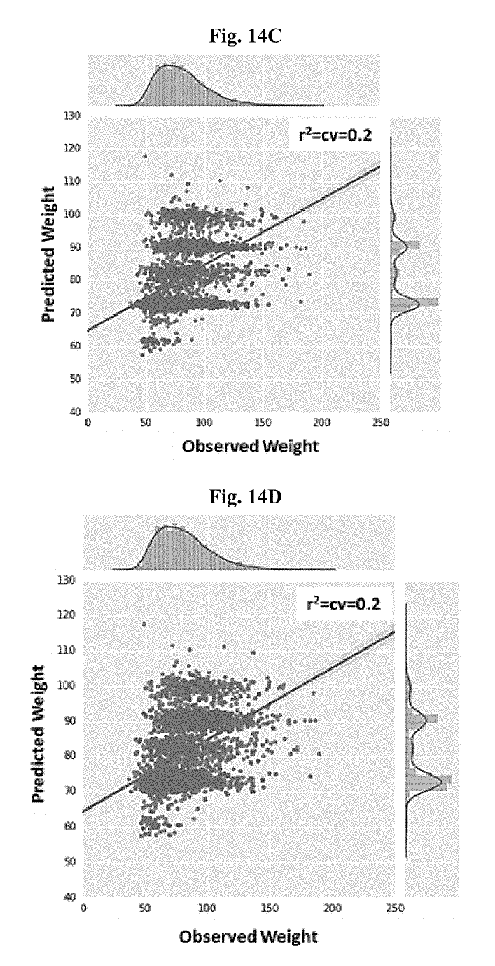

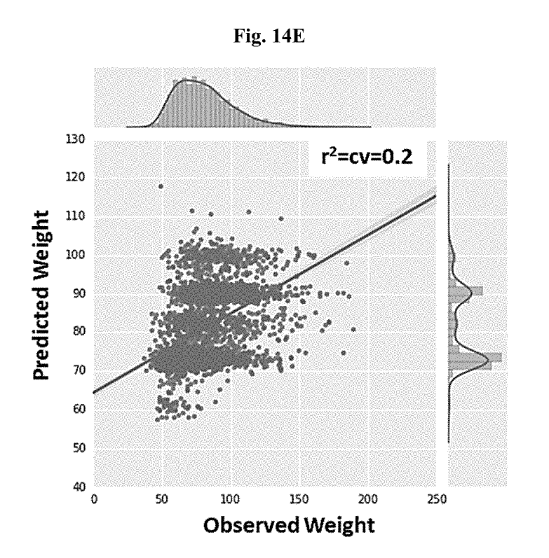

Table 6 and FIGS. 14A-E show the MAE and R²_{cv} between the observed and predicted weight by our model with different features. The prediction model with only age as a feature as in FIG. 14A has an MAE of 16.665 kg and R²_{cv} of 0.0056. The prediction model with age and sex as in FIG. 14B has an MAE of 14.963 kg and R²_{cv} of 0.154. The prediction model with age, sex, and the first 100 genomic PCs as in FIG. 14C has an MAE of 14.465 kg and R²_{cv} of 0.199. The prediction model with age, sex, the first 100 genomic PCs, and 696 height-associated SNPs as in FIG. 14D has an MAE of 14.432 kg and R²_{cv} of 0.202. When we also added the 96 BMI-associated SNPs to the previous model, we achieved the best predictive model in terms of MAE. The prediction model with age, sex, the first 100 genomic PCs, the 696 height-associated SNPs, and 96 BMI-associated SNPs as in FIG. 14E has an MAE of 14.429 kg and R²_{cv} of 0.202.

TABLE 6
Prediction quality for weight with different covariates in
ridge regression. The best predictive model includes age,
sex, top 100 genomic PCs and 696 height associated SNPs
and 96 BMI associated SNPs in the model.

| Covariates | R_{cv}² |
|---|---|
| Mean | −0.001 |
| Mean + age | 0.0056 |
| Mean + age + sex | 0.154 |
| Mean + age + sex + 100 PCs | 0.199 |
| Mean + age + sex + 100 PCs+ 696 SNP Height | 0.202 |
| Mean + age + sex + 100 PCs + 96 SNP BMI ++ 96 | 0.202 |
| SNP BMI | |

### Example 5-Predicting Eye Color and Skin Color from a Biologic Sample

Whereas height, weight, and BMI have complex genetic architecture and mid to high levels of heritability, eye color has been found to have a heritability of 0.98, with as few as eight single nucleotide variants determining most of the variability. Similarly, skin color has a heritability of 0.81 with only eleven genes predominantly contributing to pigmentation.

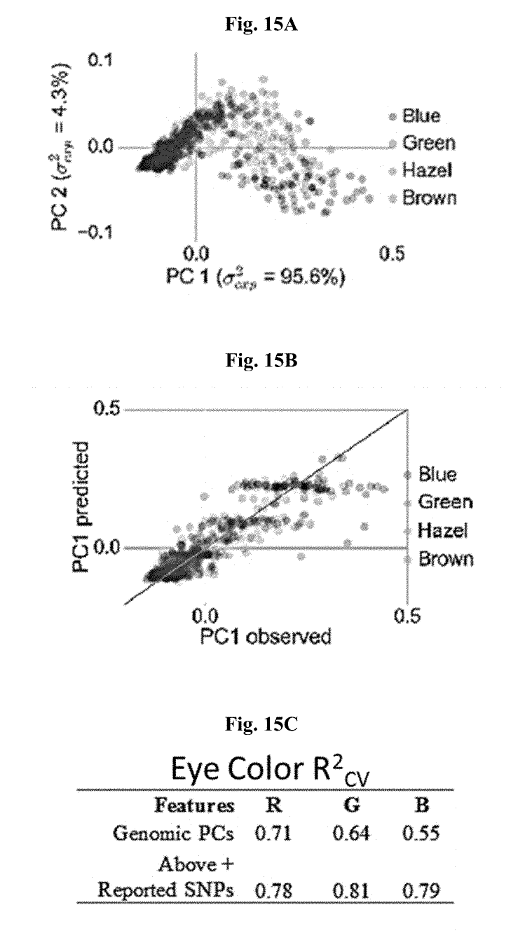

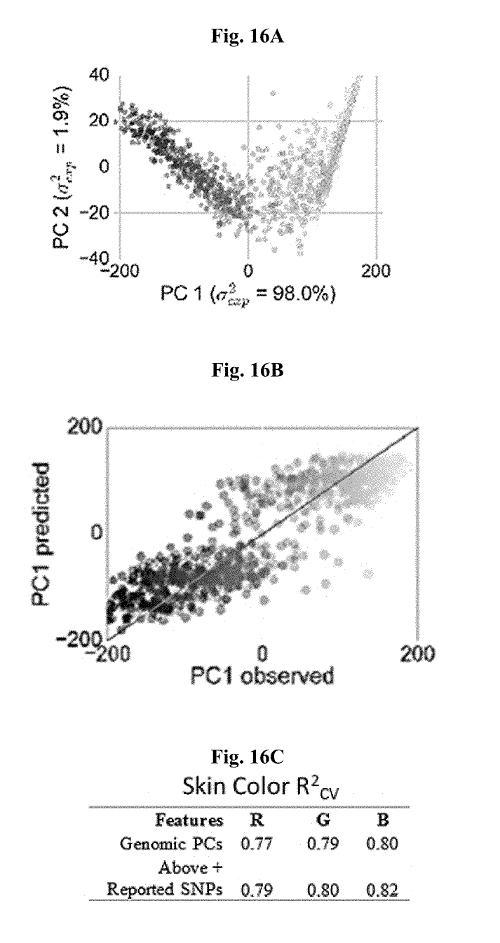

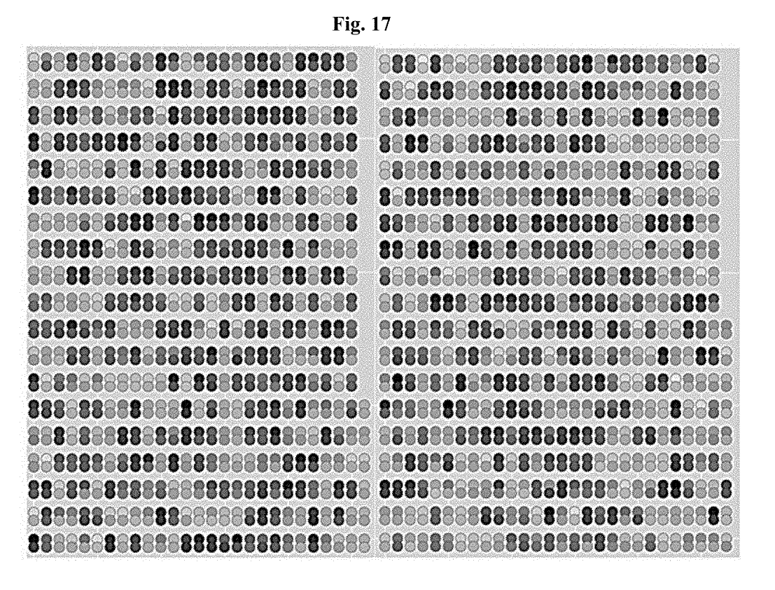

For both eye color and skin color, previous models have predicted color categories rather than continuous values. Several models predict color categories using only ad hoc decision rules, and none have used genome-wide genetic variation to predict color. In this work, we modeled both eye color and skin color as 3D continuous RGB values, maintaining the full expressiveness of the original color space as shown in FIGS. 15A-15C and FIGS. 16A-16C For both models, we calculated a high R²_{CV} of 0.78 to 0.82 for all channels.

Continuous Eye Color Prediction from the Genome

We considered genomic PCs, and SNPs as predictive features in our eye color prediction model. Since eye color varies between different ethnic groups, we included genomic PCs in our prediction model as covariates because they contain ethnic background information from the genome.

For eye color prediction, we divided our experiments into two separate analyses: 0/1/2 SNP encoding and 2-variable SNP encoding using the ridge regression model based on different covariates. First, we applied conventional SNP encoding of the minor allele dosage as 0/1/2. However, some variants associated with eye color exhibit significant dominance effects. If a set of SNPs has dominance effects on eye color, the prediction was improved when we modeled the SNPs with 2 different features: one representing the heterozygous SNP and another representing the homozygous alternate. This model is known as the 2-variable SNP encoding. We observed that 2-variable SNP encoding representations improve the prediction accuracy (Table S10).

We built 3 independent prediction models for the red (R), green (G), and blue (B) channels from the RGB color space for the 2 different encodings. We also performed a GWAS experiment to discover additional significantly associated variants beyond these published results. We did not identify additional variants other than those previously reported.

We initially considered age, sex, genomic PCs, and SNPs as predictive features in our model. A previous study found that a correlation between age and eye color for younger subjects in a specific population exists. However, our study includes only subjects ≥18 years of age, and we did not find that age was a significant determinate. Thus, we dropped age as a feature from our model. Since eye color clearly varies between different ethnic groups, we included 3 genomic PCs in our prediction model as covariates because they capture the majority of the ethnic variation from the genome. The “Self-reported eye color” covariate represents the prediction from the self-reported eye color. Due to the low prediction, these results suggest that our model can predict eye color more accurately from genetic data than can be obtained by asking people to report their own eye color.

Previous research found a set of genetic variants associated with eye color. For example, Mushailov et al. identified 5 SNPs and Walsh et al. identified 21 SNPs significantly associated with eye color. We identified 65 SNPs in the literature that produced fair predictions (see List A; they include all of the SNPs in List B minus the 5 SNPs of Mushailov et al. and overlapping SNPs in List C); 98 SNPs that produced good results (see List B); and 241 SNPs that produced good predictions (see List C) (Table 22).

We built three independent prediction models for R, G, and B from RGB color space. Table 7 shows our prediction accuracy results for each R, G, B with different covariates.

TABLE 7
Comparison of RGB eye color prediction accuracy with different covariates
in ridge regression under two SNP encodings. Using the 2-variable SNP
encoding and 3 genomic PCs, 5 known eye color SNPs, and 9 interactions
was the best model for eye color prediction in the RGB space. The lists
of the SNPs used in these models are identified in Table 22.

| | 0/1/2 SNP Encoding | | | Two Variable SNP Encoding | | |
|---|---|---|---|---|---|---|
| Covariates | R: R_{cv}² | G: R_{cv}² | B: R_{cv}² | R: R_{cv}² | G: R_{cv}² | B: R_{cv}² |
| Age | 0.010 | 0.006 | 0.002 | 0.010 | 0.006 | 0.002 |
| Sex | 0.023 | 0.022 | 0.018 | 0.023 | 0.022 | 0.018 |
| Ethnicity (3 PCs, 2 SNPs) | 0.710 | 0.644 | 0.555 | 0.710 | 0.644 | 0.555 |
| 3 PCs, 5 SNPs, 9 | 0.788 | 0.792 | 0.749 | 0.786 | 0.811 | 0.793 |
| interactions | | | | | | |
| 241 SNPs | 0.737 | 0.696 | 0.614 | 0.714 | 0.744 | 0.703 |
| 5 SNPs | 0.727 | 0.734 | 0.671 | 0.742 | 0.792 | 0.777 |
| 65 SNPs | 0.635 | 0.564 | 0.495 | 0.636 | 0.586 | 0.529 |
| 98 SNPs | 0.764 | 0.740 | 0.674 | 0.767 | 0.795 | 0.770 |
| Self-reported eye color | 0.554 | 0.721 | 0.758 | 0.554 | 0.721 | 0.758 |
| 21 SNPs | 0.743 | 0.741 | 0.674 | 0.748 | 0.798 | 0.778 |

We included three genomic PCs, five eye color associated SNPs (rs12896399, rs6119471, rs16891982, rs12913832 and rs12203592), and excluded age and sex. Since eye color is associated to ethnicity, we chose three PCs because they captured the majority of the variation in ethnicity in our dataset. The model with ethnicity covariate includes three genomic PCs, which mainly represents the genomic signal for ethnicity. We also used two variable SNP representations, where one variable encodes heterozygosity and the other encodes homozygosity. Due to low prediction efficacy with self-reported eye color, these results suggest that our model can predict eye color more accurately from genetic data than can be obtained by asking people to report their own eye color. If an SNP has dominant effects on eye color, the prediction was improved if we model the dominance effects instead of conventional SNP encoding of minor allele dosage such as 0/1/2. To do this, we model the SNP value with two different features: one representing the heterozygous SNP and another representing homozygous alternate. We observed that two-variable SNP encoding representations improves the prediction accuracy as shown in Table 7.

### Categorical Genomic Eye Color Prediction of Participants in the Personal Genome Project

For the participants of the Personal Genome Project, we had no control over the collection of phenotypes. As the facial images downloaded from the web had variable lighting conditions and one participant was wearing glasses, we decided to obtain categorical eye colors by independently asking ten human callers to determine the eye color from the photographs shown in Table 8. The resulting distribution over phenotypes was interpreted as a multinomial probability distribution over true eye color. For prediction, we first predicted continuous eye color from the genome using the same model as described above. We then mapped the continuous predictions to categorical predictions “blue,” “brown,” “green,” and “hazel.” We used a k-nearest neighbor predictor on our study population to map the predicted continuous values to the self-reported categories. The parameter k in the nearest neighbor classifier was trained using cross validation on our study cohort. The extracted continuous values for eye color were used as an input and the corresponding self-reported eye color as an output. The fraction of neighbors within each category was predicted as the probability of that category. We also report a comparison of observed and predicted eye color proportions on the PGP-10 participants in Table 9.

TABLE 8
Eye color distribution for each PGP-10 participant determined by 10 human callers.
Possible values were: blue, green, hazel, and brown. The results from each caller
are listed. The resulting eye color is quite variable for some individuals.

| | Callers | | | | | | | | | |
|---|---|---|---|---|---|---|---|---|---|---|
| | 1 | 2 | 3 | 4 | 5 | 6 | 7 | 8 | 9 | 10 |
| PGP1 | hazel | hazel | blue | blue | hazel | green | green | blue | hazel | reen |
| PGP2 | green | green | hazel | brown | brown | brown | brown | brown | brown | hazel |
| PGP3 | brown | brown | brown | brown | brown | brown | brown | brown | brown | brown |
| PGP4 | brown | brown | hazel | brown | brown | brown | brown | brown | brown | brown |
| PGP5 | blue | blue | blue | blue | blue | blue | blue | blue | blue | blue |
| PGP6 | blue | blue | blue | blue | blue | blue | blue | blue | blue | blue |
| PGP7 | brown | green | hazel | hazel | brown | green | brown | brown | hazel | brown |
| PGP8 | blue | hazel | blue | blue | blue | blue | green | blue | blue | brown |
| PGP9 | blue | blue | blue | blue | blue | blue | blue | blue | blue | blue |
| PGP10 | brown | brown | brown | brown | brown | brown | brown | brown | brown | brown |

TABLE 9
Comparison of observed and predicted distributions of eye color.
Observed proportions are computed as the fraction of human callers
choosing a given category. Predicted proportions are determined as
the fraction of nearest neighbors from our cohort that reported a
given category in the space of continuous genomic predictions.

| | Observed proportions | | | | Predicted proportions | | | |
|---|---|---|---|---|---|---|---|---|
| | hazel | green | blue | brown | hazel | green | blue | brown |
| PGP1 | 0.40 | 0.30 | 0.30 | 0.00 | 0.27 | 0.10 | 0.63 | 0.00 |
| PGP2 | 0.20 | 0.20 | 0.00 | 0.60 | 0.19 | 0.00 | 0.00 | 0.81 |
| PGP3 | 0.00 | 0.00 | 0.00 | 1.00 | 0.19 | 0.00 | 0.00 | 0.81 |
| PGP4 | 0.10 | 0.00 | 0.00 | 0.90 | 0.00 | 0.00 | 0.10 | 0.90 |
| PGP5 | 0.00 | 0.00 | 1.00 | 0.00 | 0.09 | 0.10 | 0.81 | 0.00 |
| PGP6 | 0.00 | 0.00 | 1.00 | 0.00 | 0.18 | 0.10 | 0.72 | 0.00 |
| PGP7 | 0.30 | 0.20 | 0.00 | 0.50 | 0.27 | 0.10 | 0.00 | 0.63 |
| PGP8 | 0.00 | 0.10 | 0.70 | 0.10 | 0.18 | 0.10 | 0.72 | 0.00 |
| PGP9 | 0.00 | 0.00 | 1.00 | 0.00 | 0.10 | 0.00 | 0.90 | 0.00 |
| PGP10 | 0.00 | 0.00 | 0.00 | 1.00 | 0.00 | 0.00 | 0.00 | 1.00 |

Skin Color Prediction from Genome

Skin pigmentation varies with latitude, suggesting that skin color variation is likely driven by natural selection in response to UV radiation levels. While the principal genes influencing eye and hair color are now largely identified, our understanding of the genetics of skin color variation is still far from complete, especially since the fair skin color of European and East Asian populations seem to have arose independently.

In genome-wide association studies and other analyses a number of distinct genes were implicated in skin color variation, including: the MC1R, its inhibitor ASIP, OCA2, HERC2, ALC45A2, SLC24A5, and IRF4. A number of skin color prediction models were built using different subsets of SNPs, including: a 6-SNP model, See K. L. Hart et al., Improved eye- and skin-color prediction based on 8 SNPs. Croat. Med. J. 54, 248-256 (2013); a 7-SNP model, See O. Spichenok et al., Prediction of eye and skin color in diverse populations using seven SNPs. Forensic Sci. Int. Genet. 5, 472-478 (2011); and a 10-SNP model, See O. Marorfas et al., Development of a forensic skin colour predictive test. Forensic Sci. Int. Genet. 13, 34-44 (2014). However, all of the predictive models used discrete qualitative phenotypes (skin color binned as light, medium and dark or some variation thereof) and the number and ethnic distribution of the samples was limited. In addition, the applicability of some of the models was limited to homozygous genotypes, whereas heterozygous genotypes were not considered at all. Here, we sought to determine genetic features predictive for skin color across ethnic origins.

For skin color prediction, we included age and sex (both predicted from the genome), the first three PCs, which capture the ethnicity information, and seven previously identified SNPs (rs12913832, rs1545397, rs16891982, rs1426654, rs885479, rs6119471, rs12203592) as covariates. Unlike the model by Spichenok et al., seven SNPs used in the skin color prediction model are encoded as minor allele counts instead of homozygous allele representation; these SNPs along with their annotation are listed in Table 10. We mainly compared two prediction approaches: ridge regression and extreme gradient boosting. As Table 11 shows, the extreme gradient boosting model, as implemented by XGBoost, outperformed other models. The number of estimators (n_estimators), maximum depth of a tree (max_depth), subsample proportion of instances chosen to grow a tree (subsample) and step size shrinkage to prevent overfitting (eta) were tuned using cross-validation; the best performance was obtained when parameters were set to n_estimators=1000, max_depth=2, subsample=0.9, and eta=0.01. We found that the contribution of SNPs is still marginal even in our best performing model (˜1 to 3%) and most skin color variation is captured by the first three genomic PCs (including more PCs did not result in performance improvement). True versus predicted skin color for 1,022 participants is given in FIG. 17.

TABLE 10
Annotation of variants used for skin color prediction.

| SNP Gene | SNP ID | Variation Type | Correlation |
|---|---|---|---|
| HERC2 | rs12913832 | Predicted transcription | Blue/brown eye |
| | | factor binding site | color (in Europ. |
| | | for OCA2 | and Asian pop.) |
| | | | Reduced melanin |
| | | | content in cultured |
| | | | human |
| | | | melanocytes |
| OCA2 | rs1545397 | Intron | Asian pop. |
| SLC45A2 | rs16891982 | Missense | Europ. pop. |
| | | | Blue/brown eye |
| | | | color (in Europ. |
| | | | pop.) |
| | | | Reduced melanin |
| | | | content in cultured |
| | | | human |
| | | | melanocytes |
| SLC24A5 | rs1426654 | Missense | Europ. pop. |
| | | | Reduced melanin |
| | | | content in cultured |
| | | | human |
| | | | melanocytes |
| MC1R | rs885479 | Missense | Asian pop. |
| ASIP | rs6119471 | Near 5′-end, predicted | African pop. |
| | | transcription | |
| | | factor binding site | |
| IRF4 | rs2203592 | Intron | Blue/brown eye |
| | | | color (in Europ. |
| | | | pop.) |

TABLE 11
Regression results by ridge and boosted tree on skin color
in RGB space from different combination of age, sex,
ethnicity and 7 SNPs reported from Spichenok et al.

| Model | Covariates | R: R_{cv}² | G: R_{cv}² | B: R_{cv}² |
|---|---|---|---|---|
| Ridge | Age | 0.124 | 0.140 | 0.138 |
| Ridge | Sex | −0.008 | −0.009 | −0.007 |
| Ridge | Ethnicity | 0.726 | 0.753 | 0.769 |
| Extreme | Ethnicity | 0.729 | 0.755 | 0.778 |
| Gradient | | | | |
| Boosting | | | | |
| Ridge | Age + Sex + | 0.767 | 0.793 | 0.793 |
| | Ethnicity | | | |
| Extreme | Age + Sex + | 0.773 | 0.795 | 0.806 |
| Gradient | Ethnicity | | | |
| Boosting | | | | |
| Ridge | Age + Sex + | 0.779 | 0.804 | 0.806 |
| | Ethnicity + SNPs | | | |
| Extreme | Age + Sex + | 0.787 | 0.805 | 0.815 |
| Gradient | Ethnicity + SNPs | | | |
| Boosting | | | | |
| Ridge | SNPs | 0.671 | 0.696 | 0.717 |
| Extreme | SNPs | 0.683 | 0.708 | 0.738 |
| Gradient | | | | |
| Boosting | | | | |

The shape of the human face is genetically determined as evident from the facial similarities between monozygotic twins or closely related individuals. The heritability estimates of craniofacial morphology range from 0.4 to 0.8 in families and twins. Liu et al. reported 12 SNPs influencing facial morphology in Europeans. See F. Liu et al., A Genome-Wide Association Study Identifies Five Loci Influencing Facial Morphology in Europeans. PLoS Genet. 8, (2012). Claes et al. employed a new partial least squares regression method, called “bootstrapped response-based imputation modeling”, to model variation of the face, and found 24 SNPs from 20 craniofacial genes in individuals from three West African/European admixed populations correlated with face shape. See P. Claes et al., Modeling 3D Facial Shape from DNA. PLoS Genet. 10, (2014). Despite this, the genetic features responsible for craniofacial morphology remain unknown.

Prediction of facial structure from the genome could provide a direct way to identify images from genetic information. To predict faces from the genome, we represented intra-individual face shape and texture variation using principle component (PC) analysis to define a low-dimensional embedding of the face. Next, we predicted each face PC separately using ridge regression with genomic PCs, sex, BMI, and age as covariates. We undertook a similar procedure using distances between 3D landmarks. We tested various models including ridge regression, lasso, ridge regression with stability selection, extreme boosted trees, support vector regression, neural network, and k-nearest neighbor models. Among them, ridge regression's is as good as or better than the others. The cross-validated results for different combinations of covariates predicted from the genome are given in Table 12 (Depth) and Table 13 (Color) and for true covariates are given in Table 14 (Depth) and Table 15 (Color). Unexpectedly, sex, genomic ancestry, and age provide the largest contributions to the accuracy of the models. We report both R²_{cv} as well as s₁₀ numbers.

TABLE 12
Face depth. Cross-validated results for different combinations of covariates
predicted from the genome for 10 face depth PCs for ridge regression.
Best result are in bold. Anc = ancestry from 1000 genomic PCs.
Ancestry and sex are responsible for most of the performance gain;
predicted age added a small improvement in performance.

| Face Depth PCs, Predicted Covariates | s₁₀ | R_{cv}² |
|---|---|---|
| Sex | 0.181 | 0.170 |
| Sex + Ancestry | 0.345 | 0.290 |
| Sex + Ancestry + Age | 0.363 | 0.300 |
| Sex + Ancestry + Age + BMI | 0.361 | 0.299 |
| Sex + Ancestry + Age + Height | 0.369 | 0.303 |
| Sex + Ancestry + Age + BMI + Height | 0.368 | 0.302 |

TABLE 13
Face color. Cross-validated results for different combinations of covariates
predicted from the genome for 10 face color PCs for ridge regression.
In bold is our best result. Anc = ancestry from 1000 genomic PCs.
Ancestry has the largest contribution to the model performance;
predicted sex and then age add incremental gains.

| Face Color PCs, Predicted Covariates | s₁₀ | R_{cv}² |
|---|---|---|
| Sex | 0.150 | 0.018 |
| Sex + Ancestry | 0.339 | 0.740 |
| Sex + Ancestry + Age | 0.350 | 0.744 |
| Sex + Ancestry + Age + BMI | 0.350 | 0.742 |
| Sex + Ancestry + Age + Height | 0.356 | 0.745 |
| Sex + Ancestry + Age + BMI + Height | 0.356 | 0.744 |

TABLE 14
Cross-validated results for different combinations of covariates
(age, sex, BMI and height are phenotyped) for 10 face depth PCs
for Ridge Regression. In bold is our best result. Sex-is gender,
Anc-is ancestry from 1000 genomic PCs, Age-is age, BMI-is BMI,
height is height. Ancestry and sex are responsible for most of the
performance gain, phenotyped age, BMI, and height added
small improvement in performance.

| Face Depth PCs, True Covariates | s₁₀ | R_{cv}² |
|---|---|---|
| Sex | 0.182 | 0.170 |
| Sex + Ancestry | 0.346 | 0.290 |
| Sex + Ancestry + Age | 0.391 | 0.313 |
| Sex + Ancestry + Age + BMI | 0.448 | 0.366 |
| Sex + Ancestry + Age + Height | 0.403 | 0.346 |
| Sex + Ancestry + Age + BMI + Height | 0.464 | 0.402 |

TABLE 15
Cross-validated results for different combinations of covariates
(age, sex, BMI and height are phenotyped) for 10 face color PCs
or Ridge Regression. In bold is our best result. Sex-is gender,
Anc-is ancestry from 1000 genomic PCs, Age-is age, BIM-is BMI,
height is height. Ancestry has largest contribution to the model
performance, phenotyped gender and then age add incremental gains.

| Face Color PCs, True Covariates | s₁₀ | R_{cv}² |
|---|---|---|
| Sex | 0.150 | 0.018 |
| Sex + Ancestry | 0.339 | 0.740 |
| Sex + Ancestry + Age | 0.370 | 0.744 |
| Sex + Ancestry + Age + BMI | 0.374 | 0.744 |
| Sex + Ancestry + Age + Height | 0.370 | 0.745 |
| Sex + Ancestry + Age + BMI + Height | 0.375 | 0.744 |

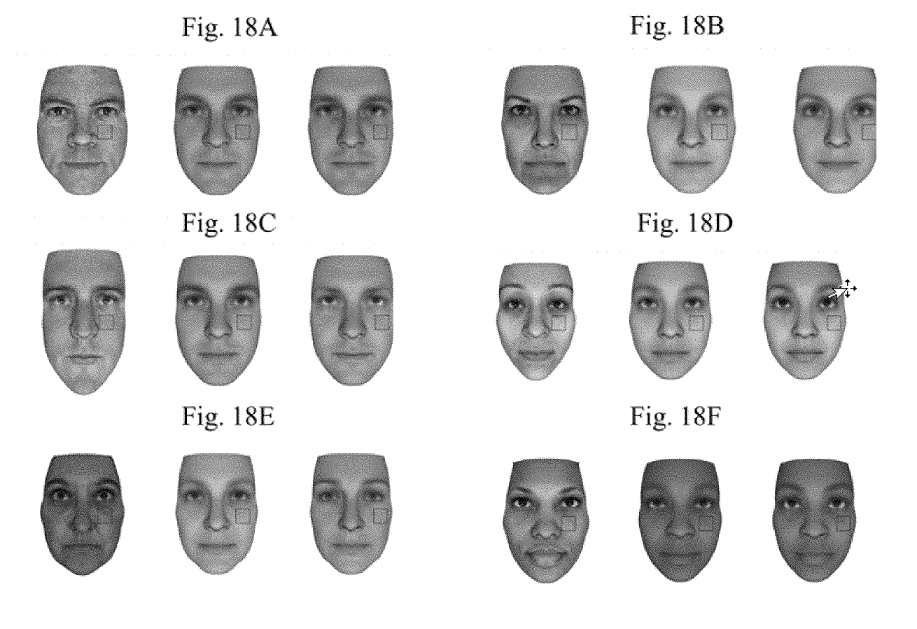

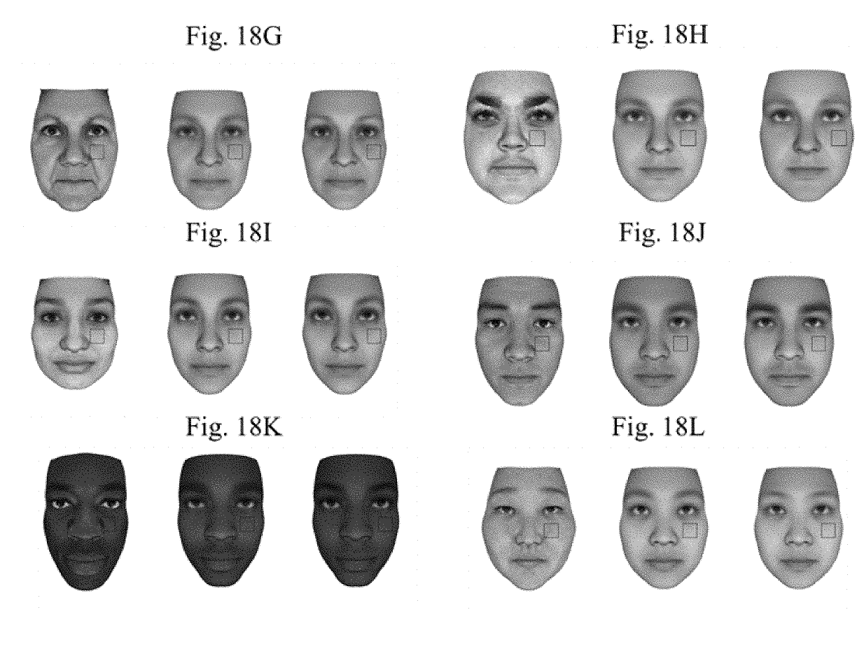

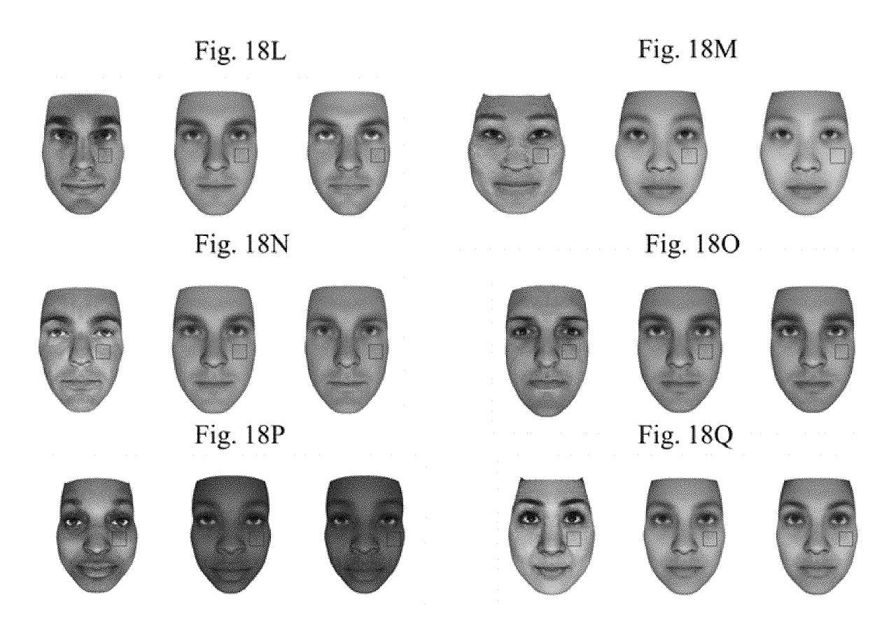

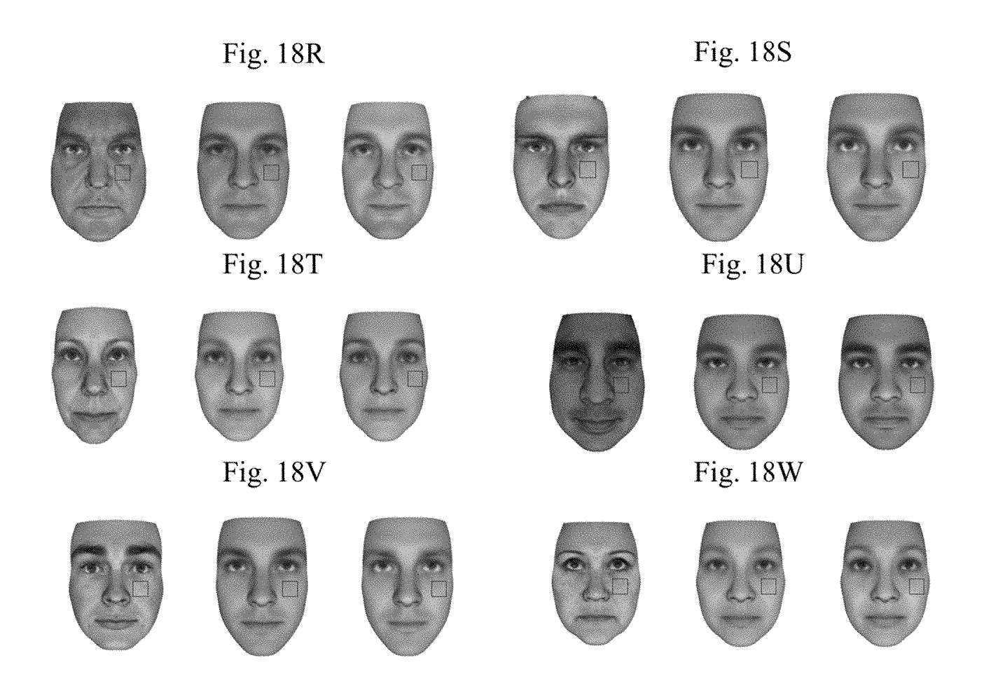

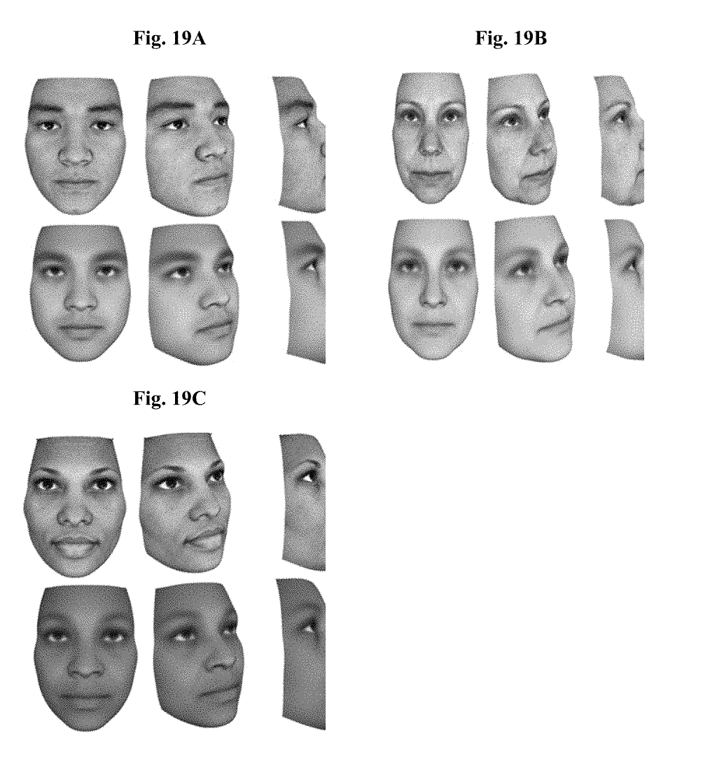

True faces next to predicted faces by both Ridge and k-Nearest Neighbor methods for 24 consented individuals that were assigned to the holdout set are given in FIGS. 18A-18W. 3D faces of three selected individuals from the holdout set scanned and predicted using Ridge regression are provided in FIGS. 19A-19C.

We observed that facial predictions accurately reflected the sex and genetic ancestry of the individual. For Africans, predicted faces qualitatively reflected the overall variation in face shape. For Europeans, predictions were more homogeneous. For this group, we found 1.4 to 2.7-fold lower standard deviation in predicted PCs as shown in Table 16.

TABLE 16
The ratio of standard deviation of African ancestry (STD_AFR)
to standard deviation of European ancestry (STD_EUR) for ten
face depth PCs. Among 10 PCs, 9 of the STD_AFR/STD_EUR
are >1.00, which indicates a larger facial variability in
African ancestry tha European ancestry.

| Predicted face shape | STD_AFR/STD_EUR |
|---|---|
| PC 5 | 2.80 |
| PC 2 | 1.61 |
| PC 8 | 1.57 |
| PC 9 | 1.46 |
| PC 10 | 1.27 |
| PC 3 | 1.26 |
| PC 7 | 1.22 |
| PC 4 | 1.12 |
| PC 1 | 1.01 |
| PC 6 | 0.89 |

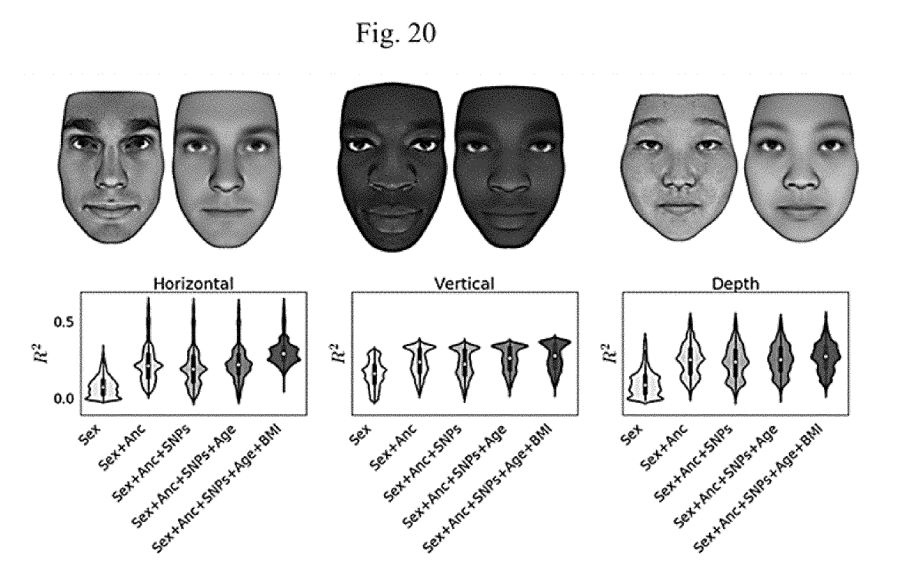

To assess the influence of each covariate on predictive accuracy, we measured the per-pixel R²_{CV} between observed and predicted faces. Since errors were anisotropic, we separated residuals between horizontal, vertical, and depth dimensions. FIG. 20 shows the distribution of predictive accuracies along each axis as a function of the covariates used in the model. Surprisingly we observed from this plot that sex and genetic ancestry alone explained large fractions of the predictive accuracy of the model. Previously reported single nucleotide polymorphisms (SNPs) related to facial structure did not improve the sex and genetic ancestry model for any region of the face. In contrast, we found that both age and BMI improved the accuracy of facial structure along the horizontal and vertical dimensions.

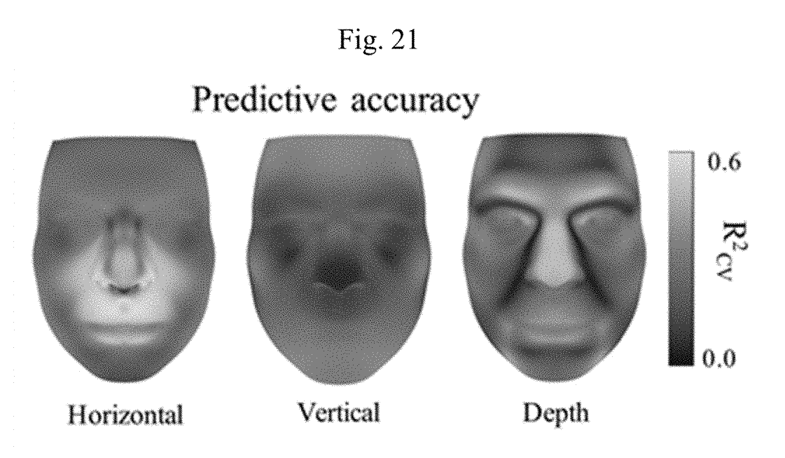

To further understand predictive accuracy for the full model, we mapped per-pixel accuracy onto the average facial scaffold FIG. 21. Much of the predictive accuracy along the horizontal dimension came from estimating the width of the nose and the lips. Along the vertical dimension, we obtained the highest precision in the placement of the cheekbones and the upper and lower regions of the face. For the depth axis, the most predictable features were the protrusions of the brow, the nose, and the lips. To examine the effect of ethnicity on variability in face shape predictions, we created a group of individuals with >80% African (AFR) ancestry and >80% European (EUR) ancestry. Table 16 presents the AFR:EUR ratio of the standard deviation for each of the first 10 face depth PCs. This demonstrates that predictions were more variable for those with high African ancestry than those with high European ancestry.

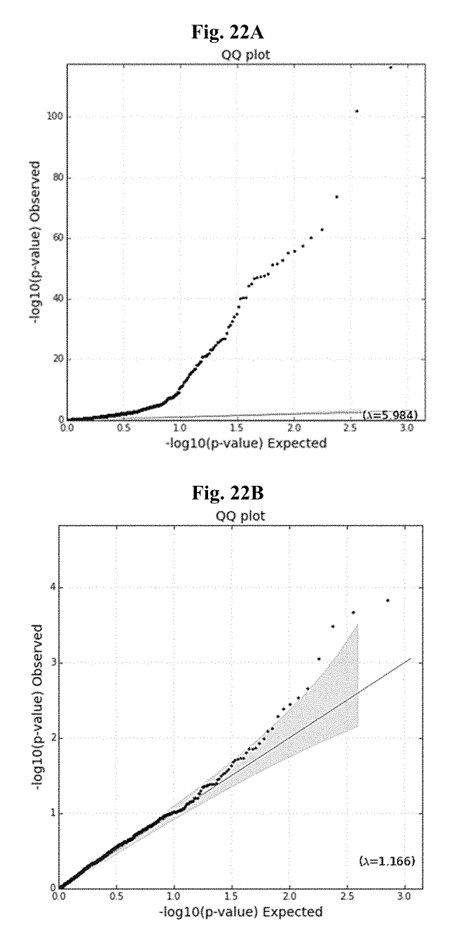

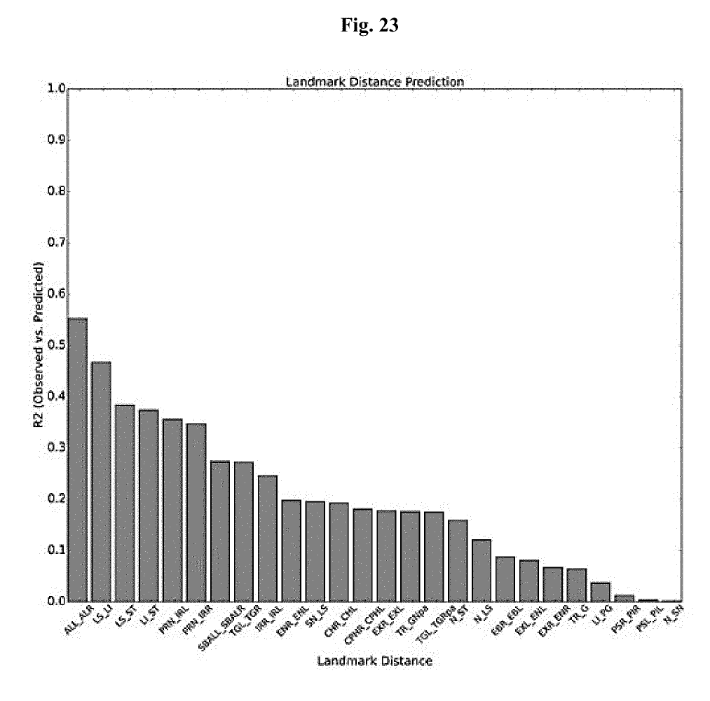

To investigate SNPs associated with the face shape and color, we have performed association testing between the top 10 PCs from our face depth and color embedding and the reported SNPs. When we tested for the associations having sex, BMI, and age as covariates, the genomic control inflation factor λ on this set of tests was 5.96, which indicates strong confounding effects in the tests. The λ statistic is defined as the ratio of the median of observed statistic to the median of the expected statistic under null distribution, and λ>1 indicates an inflation of statistics due to confounding. In our analysis, we found strong indication for confounding by population structure. After adding 5 ethnicity proportions as covariates, λ dropped to 1.15. At an alpha level of 0.05, none of the 36 candidate SNPs were significant after Bonferroni correction (P<7×10⁻⁵). The corresponding Quantile-Quantile (Q-Q) plots are shown in FIGS. 22A and 22B.

Landmark Distance Prediction from Genome

Researchers have studied landmark distances for various purposes including craniofacial anomaly detection and facial growth analysis, and have attempted relate landmark distances to the genome. Paternoster et al. has shown that the nasion position is associated with a SNP in PAX3 for 2,185 adolescents, which has been replicated by another set of cohorts with 1,622 individuals. See L. Paternoster et al., Genome-wide Association Study of Three-Dimensional Facial Morphology Identifies a Variant in PAX3 Associated with Nasion Position. Am. J. Hum. Genet. 90, 478-485 (2012). GWAS have identified 5 candidate genes affecting normal facial shape variation in landmark distances for Europeans, PRDM 16, PAX3, TP63, C5orf50, and COL17A1 combined 12 SNPs, were identified as genome-wide significant. However, the SNP explains only 1.3% of the variance of nasion position, and associations between diverse landmark distances and genome are largely unknown.

To understand the genetic architecture of facial landmark distances, we performed a GWAS experiment on 27 face landmark distances. Each of the 27 landmark distances were measured for 1,045 individuals for which 3D images of sufficient quality were obtained. For SNP data, we collected 30 million SNPs for 1,045 individuals from WGS data. After applying a MAF threshold of 5% and missingness threshold of 10%, 7,098,585 SNPs were used for GWAS analysis. We performed two different approaches for GWAS analysis: linear regression and linear mixed model regression. For the linear regression model, we included the first five genomic PCs as covariates to account for population structure. For both approaches, we included age and sex as covariates in the GWAS analysis. As shown in Table 17, we found two novel genome-wide significant hits from two face landmark distances. This was obtained after applying both a genome-wide significance threshold of 5×10⁻⁸ and a phenotype-specific permutation p-value threshold. One significantly associated SNP (rs7831729, p-value: 9.67×10⁻¹⁰, permutation threshold: 2.22×10⁻⁸) for the height of left eye (PSL_PIL) is replicated for right eye (PSR_PIR, p-value: 3.57×10⁻⁸, permutation threshold: 1.82×10⁻⁸). This replication supports the association between SNP and the height of the eye. To obtain the permutation p-value threshold, we first performed GWAS analysis on permuted phenotypes to find the minimum p-value from GWAS. The permutation p-value threshold is then computed by multiplying 0.05 by the minimum p-value from permuted GWAS for each phenotype. This corresponds to the Bonferroni correction since this cutoff controls the probability of including at least one false finding.

TABLE 17
Two SNPs significantly associated with 2 landmark distances out of the 27
landmark distances in our GWA analysis. One significantly associated SNP (rs7831729)
for the height of left eye (PSL_PIL) is replicated for right eye (PSR_PIR).
LMM = linear mixed model regression. linreg = linear regression

| | | | | Permutation | | |
|---|---|---|---|---|---|---|
| | | | | P-value | Landmark | GWAS |
| SNP (rsid) | Chr | Position | P-value | Threshold | Trait | method |
| rs1371563 | 2 | 119062468 | 1.74 × 10⁻⁸ | 3.18 × 10⁻⁸ | LS_ST | LMM, linreg |
| rs7831729 | 8 | 100664344 | 9.67 × 10⁻¹⁰ | 2.22 × 10⁻⁸ | PSL_PIL | LMM, linreg |
| | | | 3.57 × 10⁻⁸ | 1.82 × 10⁻⁸ | PSR_PIR | LMM, linreg |

We evaluated the performance of prediction of landmark distances from genomic information, predicted sex from the genome, predicted age from the genome, and the top 3 genome PCs) using R²_{cv} between observed and predicted landmark distances. In FIG. 23 ALL_ALR (width of nose) and LS_LI (height of lip) are the most predictive, while N_SN (length of nose) and PSL_PIL/PSR_PIR (height of the left/right eye) are the least predictive. The results agree with our observation that the width of the nose and the height of the lip are excellent features to distinguish between different ethnicities. However, the length of the nose and the height of the eyes vary greatly within ethnicities. Thus, it is difficult to predict them from genome given our limited sample size.

### Example 7-Predicting Voice Pitch from a Biologic Sample

For prediction of voice, we extracted and predicted a 100-dimensional i-vector and voice pitch embedding from voice samples collected from our cohort. Similar to face prediction, we fit ridge regression models to each dimension of the embedding. As covariates, we used the first ten genomic PCs and sex. We were able to predict voice pitch with an R²_{CV} of 0.70. However, predictions for only three of the 100 identity-vector dimensions exceeded an R²_{CV} of 0.10.

While direct prediction of face and voice from the genome is valuable, for re-identification purposes, it may be more efficient to explicitly extract informative and well-predicted traits such as age, sex, and ethnicity from these observations. Such phenotypes, extracted from the face and voice, may then be matched to those predicted from the genome. To leverage these benefits, we therefore predicted age, sex, and ethnicity from observed faces and voice samples Table 18.

To quantify how well face and voice capture information about age, sex and five regions of ancestry, we predicted these traits from observed face depth, face color, landmark distances, and voice i-vectors using ridge regression. As input features for prediction from face depth and color we used 200 of the corresponding PCs. As input features for prediction from voice, we used all 100 available i-vectors and voice pitch. Similarly, we used all landmark distances for prediction. This approach is helpful for extracting demographic information from face and voice where such information is useful but not otherwise accessible. In addition, it leads to higher select and performance compared to directly matching observed to predicted values for face and voice.

We show that face shape, face color, and voice are reasonably predictive of age, sex, and ancestry. In summary, we are able to predict face and voice from the genome and to programmatically extract age, sex, and ethnicity with reasonable accuracy by examining face and voice embeddings. Both of these approaches may be useful for forensic casework.

TABLE 18
R²_{CV} values for age, sex, and five region genetic
ancestry from face shape, face color, and voice.

| | Age | Sex | AFR | EUR | EAS | AMR | CSA |
|---|---|---|---|---|---|---|---|
| Face Shape | 0.82 | 0.79 | 0.84 | 0.78 | 0.57 | 0.16 | 0.11 |
| Face Color | 0.75 | 0.84 | 0.89 | 0.84 | 0.62 | 0.24 | 0.24 |
| Voice | 0.62 | 0.70 | 0.67 | 0.38 | 0.14 | 0.03 | 0.02 |

### Example 8—Re-Identification of Individuals from a Biological Sample

In the previous examples, we presented predictive models for face, voice, age, height, weight, BMI, eye color, and skin color. We integrated each of the individually informative phenotypic predictions according to the approach outlined in FIG. 2. We predicted an array of traits from the genome alone and ranked the observed faces by their similarity to these predictions. Face prediction was modified to use genomic predictions of sex, BMI, and age rather than observed values. Finally, to account for variations in prediction quality, we adapted a maximum entropy classifier to learn an optimal distance metric between observed and predicted values for each feature set.

To assess the performance of adaptive phenotypic prediction-based ranking, we considered the following task. Given an individual's genomic sample, we sought to identify that individual out of a pool of size N. For example, given forensic biological evidence, we would attempt to pick the correct individual out of a pool of N suspects. We refer to this problem as select at N (s_{N}). We also considered a second scenario wherein N de-identified genomic samples were matched to N phenotypic sets such as those that could be gleaned from online images and demographic information. This corresponds to post-mortem identification of groups or re-identification of genomic databases. We refer to this challenge as match at N (m_{N}).

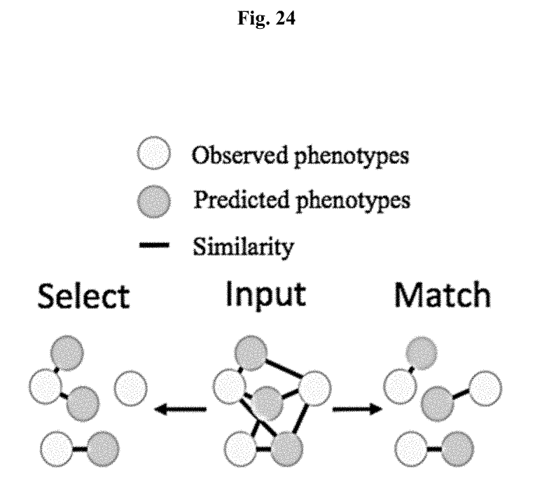

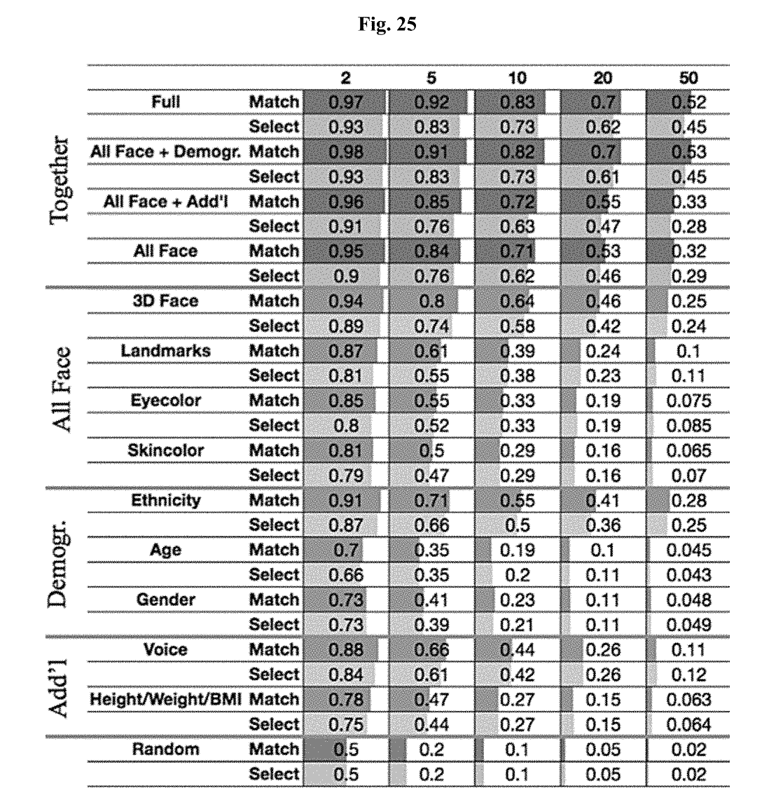

FIG. 24 presents a schematic of the difference between s_{N} and m_{N}. For s_{N}, genomes are paired to the phenotypic profile that they best match, based on the model described in the previous section. In contrast, we treated m_{N} as a bipartite graph matching problem wherein total likelihood of correct pairs was maximized across the graph. That is, each genomic sample is linked to one and only one individual in a globally optimal manner. FIG. 25 presents the performance of s_{N} and m_{N} across features sets and pool sizes.

In particular, we consider three sets of information: 1) 3D face, 2) demographic variables such as age, gender, and ethnicity, and 3) additional measurements like voice, height, weight, and BMI. Surprisingly, we found that 3D face alone was highly informative, with a s₁₀ value of 58%; this is more than a five-fold improvement over baseline. We found that ethnicity was the second most informative feature, with an s₁₀ performance of 50%. Voice had comparable performance to ethnicity, while age, height/weight/BMI, gender, and age all yielded s₁₀ performance of around 20%. Finally, we integrated these variables to obtain s₁₀ performance of 77%. For the full model, m₁₀ performance was 82%, compared to 62% for 3D face alone.

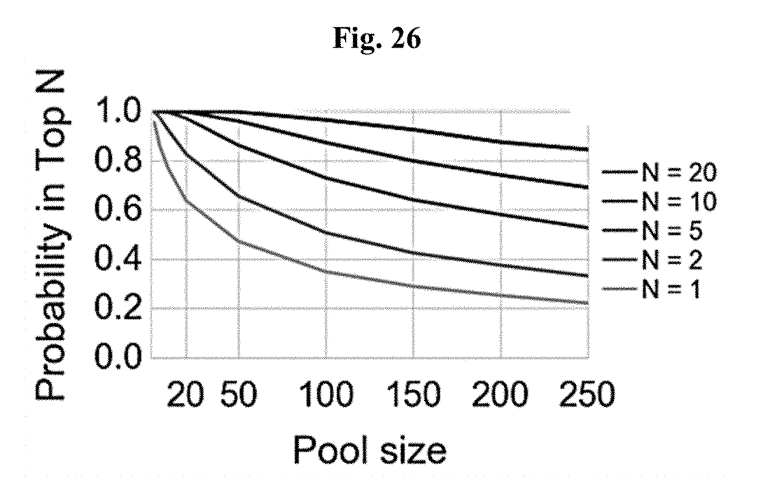

Of use for forensic applications is the ability to intelligently select a reduced pool of individuals such that law enforcement resources are maximized. FIG. 26 presents our ability to ensure that an individual is in the top N from an out-of-sample pool of size M>N. An example scenario is the probability of including the true individual in a 10-person subset of a random 100-person pool chosen from our cohort. Using our current data, we include the correct individual in the top ten 88% of the time. Therefore, this method provides the potential to significantly enrich for persons of interest.

### Evaluation Metrics for Individual Re-Identification

To assess the effectiveness of our models for the individual re-identification task, we evaluated our predictions using two performance metrics, referred to as select at N (s_{N}) and match at N (m_{N}). sₙ is defined as the accuracy in picking a genomic query's corresponding phenotype entity out of a pool of size N. mₙ represents the task of uniquely pairing N queries to N corresponding phenotype entities. The features for sₙ and mₙ are the average absolute differences between each observed trait set and each predicted traits set generated by the predictive models. Between feature sets (e.g., face shape, eye color, etc.) the number of individual variables may be quite different. Residuals are averaged across the variables of a feature set to ensure that the influence of a feature set was not correlated with the number of variables within it. The following is the general procedure for both sₙ and mₙ algorithms: 1) generate training data, where input data are the absolute residuals of predicted and observed traits; 2) use training examples from matching and non-matching pairs to learn weights on absolute residuals for each feature set; 3) using these weighted distances between observed and predicted traits, generate the probability that a given observed/predicted pair belong to the same individual; 4) place these probabilities as edge weights on a graph; and, 5) choose the node(s) that satisfy the select or match criteria, respectively. In Select, we simply pick the entity in the pool that has the highest probability of matching the probe. For Match, we choose all pairs so as to maximize the total probability of matching within the set of N pairs. This is performed using the blossom method, as implemented by the “max weighted matching” function from the Python package NetworkX.

As described in 2), the “probability of match” classification model was fitted using matching and non-matching pairs as training examples. For models that included sex, this variable was treated as a hard constraint. That is, pairs with discordant observed and predicted sex were assigned a matching probability of zero. Otherwise, out-of-sample predicted probabilities were produced for each pair using three-fold cross-validation. It should be noted that cross-validation for Select/Match had different folds than that of other prediction models because the Select/Match model operates on pairs of individuals instead of the individuals. We verified that our “probability of match” model was not over-fitted by comparing the distribution of match probabilities for sex-concordant pairs that came from the same versus different folds in the component predictive models. The concern is that since observations in the same fold are predicted using the same model, they may be biased towards being more similar. Since true matching pairs arise from the same individual, these values are of course predicted in the same trait prediction folds. In this way our model could be biased towards finding matches. We find that there is no significant difference between the same/different fold match probability distributions for any of the feature sets. We confirmed this by visual inspection and by Mann-Whitney U p-values. Finally, we performed our “probability of match” calculation using YASMET (available at http://www.fjoch.com/yasmet.html), a maximum entropy model.

### Feature Sets for Individual Re-Identification

Feature sets for re-identification are presented in FIG. 25. To improve the information density of extracted features for voice, landmarks, and face PCs; age, sex, and five-region genomic ancestry were predicted from each of these sets. Matching was then performed between these predicted values and the corresponding observed counterparts. For example, for 3D facial structure, directed feature extraction improved s_{N} and m_{N} performance compared to matching predicted facial PCs to their observed values. PC prediction yielded s₁₀ performance of 32%, compared to 58% for age, sex, and ethnicity extraction.

### Example 9-Re-Identification of Individuals from the Human Genome Project

To illustrate the generalizability of our analysis framework to a setting where phenotyping is not controlled, we cross-tested our approach on the first ten participants in the Personal Genome Project (PGP10). See G. M. Church, The personal genome project. Mol. Syst. Biol. 1, 2005.0030 (2005). The PGP10 is composed of eight men and two women. All but one of the participants are European. In addition to sex, genetic ancestry, skin color, eye color, and face data; we were also able to access and predict the blood group of each individual. See Table 20.

In this set, we encountered the following additional challenges. First, the available phenotypes were different from those in our own cohort. Since 3D faces were not available, we used pre-trained neural network-based predictions from two-dimensional images obtained from the web. Similarly, variability in lighting conditions significantly impeded our ability to precisely quantify color. For eye color, we obtained categorical colors via votes from ten independent raters. In addition, our age prediction model was not applicable to these data since raw read data were not available to provide information on telomere length or low frequency mosaic sex chromosome loss.

A second major challenge was that the number of individuals was not sufficient to train a new distance learning model on the modified features set. To obtain a combined distance metric without training, we simply took the mean squared error between predicted and observed values for each individual phenotypic prediction.

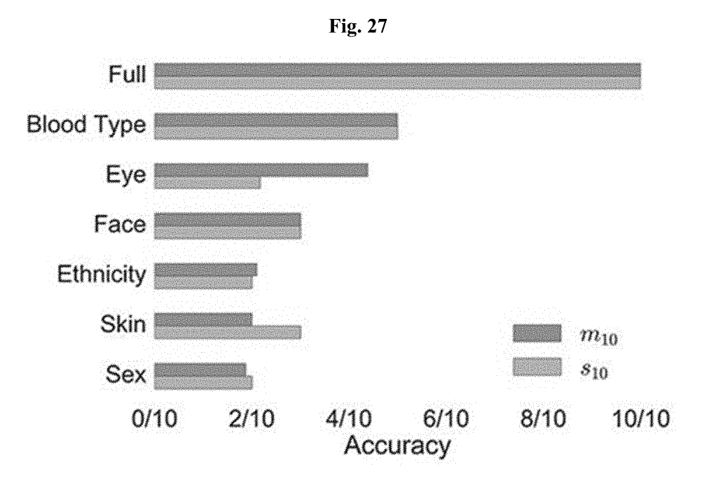

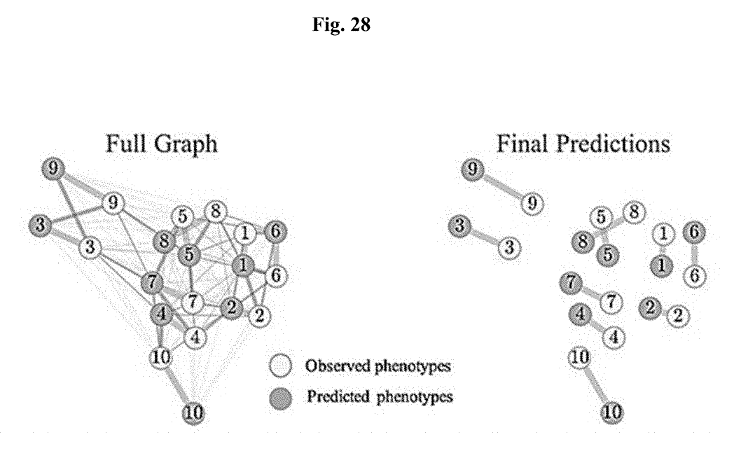

The results of the individual predictors are shown in FIG. 27. Due to greater sex and ethnicity imbalance and lower phenotyping quality for skin color, eye color, and the face image, we achieved significantly lower select and match performance for these variables compared to what we observed in our own study cohort. However, when including blood group prediction, all ten participants were ranked closest to themselves for s₁₀ and m₁₀ FIG. 28. These results demonstrate that, given a handful of informative phenotypes, our approach is generalizable to cases where distance learning is not possible and phenotypic quality is inhomogeneous.

Re-Identifying Individuals from the PGP10 Data

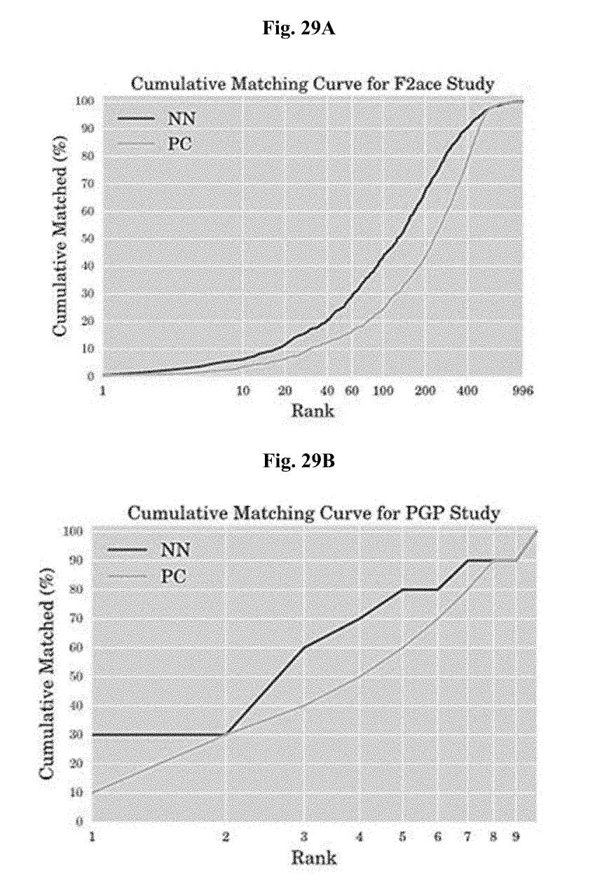

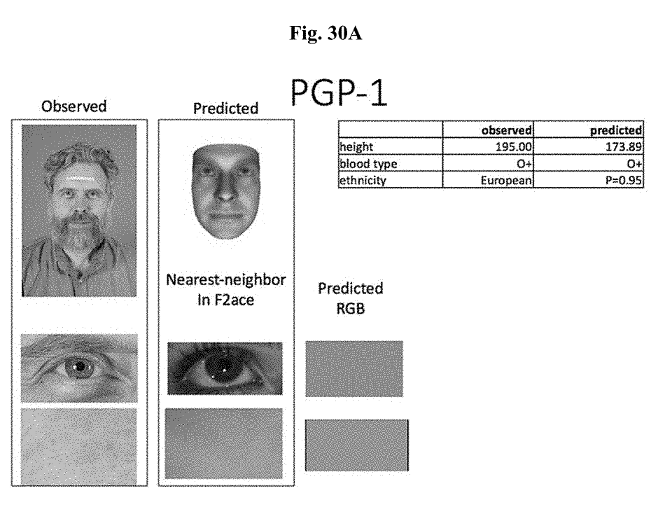

The prediction of various traits and faces of the PGP10 individuals by our models are shown in FIGS. 29A and 29B as well as FIGS. 30A-30J. We collected 6 different phenotypes: 2D facial image embedding, skin color, categorical eye color, blood type, sex, ethnicity, and height. The majority were obtained simply by reading the public records on the PGP web site (blood type was unavailable for PGP-3). However, 2D images, skin color, eye color, and height required more effort. Since the PGP-10 had frontal face images taken upon enrollment, a Google image search revealed 9 out of 10 of the original PGP face photos in SNPedia. Because of the relative high profile of the participants, we were able to fill in the last with found images. All images were released under the Creative Commons license CC BY-NC-SA 3.0 US. These photos provided skin crops from which we extracted skin color. For eye color, we asked ten human callers to label the eye color in one out of four categories “blue”, “green”, “hazel”, and “brown”. We report the distribution of obtained eye color phenotypes in Table 9. Only three participants reported their height. We estimated heights of three participants by using a group picture with five standing participants. Two participants in this image had reported heights, and we inferred the other three through simple relative measurements. The remaining four were inferred by finding pictures where they stood next to a celebrity with a public height (i.e., Salman Rushdie, Jimmy Fallon, or Bill De Blasio). Such public heights are themselves suspect. Because of the ad-hoc method, we decided that height is an untrustworthy measurement and we omitted it from further analysis.

All PGP-10 participants have one or more whole genome variant files provided by Complete Genomics aligned using reference GRCh37. We used Complete Genomics's megatools suite to convert the files to VCF4.1 format. These were lifted onto GRCh38, and filtered to remove indels. We then extracted genomic PCs in the manner described above. Finally, we predicted all phenotypes using our models, including the use of Boogie, a blood type predictor. As raw read data was not available, we were not able to estimate telomere lengths and mosaic loss of sex chromosomes for prediction of age from the genome.

Identification and Prediction from 2D Face Embedding

We used 3D face images for face prediction from genome, which requires an advanced camera setup that captures detailed 3D renderings of each individual's face. However, there are many times when 2D images are available and 3D images are not. For example, as in our experiments on PGP dataset, an enrollee's genome may be present in the PGP dataset and a 2D image may exist on Google Image search (this is how we located 2D images for the PGP10 data).

To investigate the cases where 3D images were not available, we performed s_{N} and m_{N} using only 2D images. Specifically, we investigated a variety of 2D face embeddings, and judged them on their ability to perform closed-set face identification and on their ability to be predicted from the genome. The closed-set face identification is a problem wherein one enrolls a set of face images in the system and, given a new picture of an enrolled subject, the system determines the best match to the subject's identity.

We experimented with Gaussian mixed models (GMM), local Gabor binary pattern histograms (LGBPHS), Eigenfaces (PCA), Gaborjets, and neural network embeddings. We used the Bob Face Recognition Library to explore different embeddings (except the neural network) as well as different image pre-processing steps. We used the OpenFace NN4.v1 model as our neural network embedding. This is a convolutional neural network based on the Inception network model that produces a 128-dimension vector. The model was trained by combining two large publicly-available face recognition datasets: FaceScrub and CASIA-WebFace.

In our study, each participant had a front-facing 2D face image. Among them, 106 individuals had two separate 2D images. For each embedding technique, we enrolled the subject's first image, and then used the subject's second image as a probe for the face identification task. Table 19 shows the percentage of probes that correctly identified the enrolled user. Though the GMM outperformed Gabor Jets, it used 35,840 features vs. 4,000 features for Gaborjets. Both vastly outperformed Eigenfaces in this closed-set identification task.

TABLE 19
Comparison of 2D image embeddings by re-identification
performance on 106 repeated images. The performance
of 5 different 2D face embeddings on a closed-set face
identification task with 106 subjects and 2 images per
subject. Given a new picture of an enrolled subject, the
system determines the best match to the subject's identity.
s106 measures the fraction of second images that were closest
to the first images that had been used to train the model.

| Embedding | S₁₀₆ |
|---|---|
| GMM | 83.01% |
| Gabor Jet | 75.47% |
| LGBPHS | 71.69% |
| OpenFace Neural Network | 38.67% |
| PCA (300) | 13.20% |

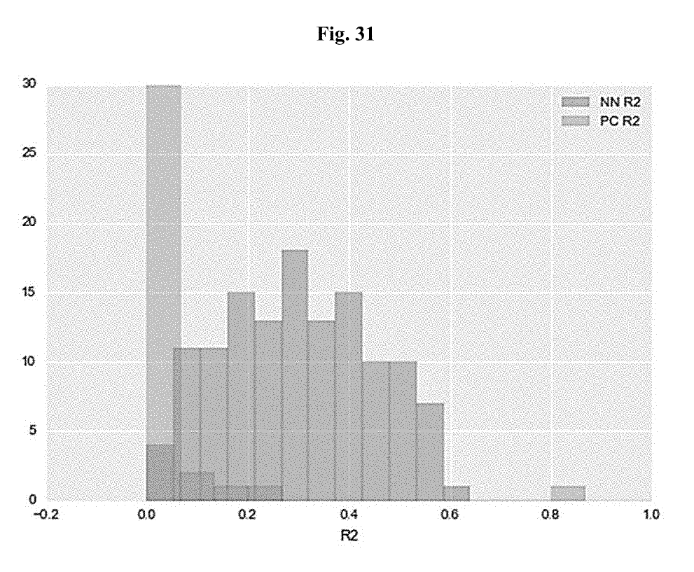

We hypothesized that Gaborjets would do well because they capture fine-grain texture information for the face. While this may work for face identification, such low-level features are not likely to be genetically predictable. In contrast, neural networks may be able to learn fundamental face structures that are related to the genome as shown in FIG. 31. The histogram of variance is explained (R²) from a Ridge regression that uses genomic features to predict each individual dimension of either the PCA or neural network embedding. In fact, while the first PC is highly predictable (0.8 R²), the majority of the other components are not. In contrast, the majority of the neural network dimensions are predictable.

To illustrate the power of having a genetically predictable embedding, we used the embeddings to perform closed-set identification. However, this time we attempted to identify all individuals from our cohort and the PGP10 participants by using either 2D face PC or neural network embeddings. The system enrolled all of the observed embeddings computed from existing 2D face pictures. We then used genetically predicted embeddings to find the best match in the enrolled observed subjects. FIG. 31 shows that the neural network embedding outperformed PCA. In fact, we were able to correctly identify 30% of the PGP10 participants with no other information.

### Example 10-Blood Group Prediction from the Genome

To predict ABO and Rh blood groups from genome, we employed the method developed by Giollo et al. with minor modifications. See M. Giollo et al., BOOGIE: Predicting blood groups from high throughput sequencing data. PLoS One. 10, e0124579 (2015). We classified the blood groups based on haplotypes. We define a haplotype by a set of SNPs in the coding regions for ABO or RhD genes on a single chromatid. First, with a set of 99 SNPs for ABO, and 64 SNPs for RhD. See S. K. Patnaik, W. Helmberg, O. O. Blumenfeld, BGMUT: NCBI dbRBC database of allelic variations of genes encoding antigens of blood group systems. Nucleic Acids Res. 40 (2012). We began by enumerating all possible haplotypes, all possible haplotypes had to be considered since we had no phasing information. Because of the small number of sites and low number of heterozygous SNPs in our dataset (e.g., ≤17 for both ABO and Rh), exhaustive enumeration was feasible. By choosing the closest match for each query using Hamming distance, chromatids were predicted as A, B, O, AB or NA (ABO group), and D+, Weak D, Partial D, D- or NA (Rh group). Finally, we sorted the chromatin pairs (pairing based on complementary bases) by the average Hamming distance of the pairs in ascending order, and then called the blood group based on rules in Table 20. Pairs of chromatins have the same distance, we broke the ties by the number of supporting haplotypes in the training dataset.

TABLE 20
Rules for determining the final blood group phenotype off the chromatid pair k-NN
predictions. Blood group phenotype prediction rules for (a) ABO and (b) Rh. The
value NA represents an ambiguous prediction, as described in the main text.

| A | | | | | | B | | | | | |
|---|---|---|---|---|---|---|---|---|---|---|---|
| Chromatids | A | B | O | AB | NA | Chromatids | D+ | _{Weak}D | _{Partial}D | D− | NA |
| A | A | AB | A | AB | NA | D+ | D+ | D+ | D+ | D+ | D+ |
| B | AB | B | B | AB | NA | _{Weak}D | D+ | _{Weak}D | _{Weak}D | _{Weak}D | _{Weak}D |
| O | A | B | O | AB | NA | _{Partial}D | D+ | _{Weak}D | _{Partial}D | _{Partial}D | _{Partial}D |
| AB | AB | AB | AB | AB | AB | D− | D+ | _{Weak}D | _{Partial}D | D− | D− |
| NA | NA | NA | NA | AB | NA | NA | D+ | _{Weak}D | _{Partial}D | D− | NA |

The 10-fold CV error for the ABO group prediction was 12.3% and for the Rh group prediction was 26.3% on the BGMUT dataset. To validate the statistical significance of our blood group predictions, we ran label permutation tests to obtain p-values for each classifier; we performed 10,000 iterations, each of which ran cross-validation on randomly shuffled labels. Permuted p-values were 9.9e-5 and 12e-5 for the ABO and Rh predictions, suggesting that both are statistically significant. We predicted correct ABO group for 81 samples (95.2% accuracy) and correct RhD group for 80 samples (94.1% accuracy). The number of samples that were predicted correctly for both ABO and RhD groups was 76 (89.4%) using the PGP dataset. Both ABO and RhD groups were predicted with 100% accuracy for the PGP-10 dataset except sample PGP3, which did not report either ABO or Rh blood group annotations. The CV accuracy on BGMUT dataset was significantly different from the test accuracy on the PGP dataset because 2 datasets have different distributions of RhD phenotypes. In PGP dataset, we calculated 11 D− samples and 74 D+ samples in PGP which reflect the Caucasian population, and in BGMUT dataset, we have 1 D+, 29 D−, 25 Partial D and nine Weak D samples. The differences in accuracy can be explained in phenotype frequency differences, as genotypes corresponding to Weak D and Partial D phenotypes are the result of few missense mutations on the D+ genotype, i.e., they are very similar to each other. Moreover, the list of haplotypes for these phenotypes in the BGMUT database is not comprehensive. The permutation procedure is less reliable and more likely to produce a chromatid pair that is closest to the wrong phenotype. After removing Partial D and Weak D phenotypes from the CV dataset, the program resulted in one error out of 14 predictions, an error rate of 7.1%, which is comparable to our PGP results.

Furthermore, we tested the robustness of our predictions as we change the number of samples in the training dataset and the number of heterozygous sites. As shown in Table 21, we found only a slight decrease of prediction accuracy for both ABO and Rh blood groups, even when halving the BGMUT haplotypes in our training dataset. We also investigated whether we made more errors on samples that had more heterozygous sites; however, we found no correlation between them. As expected, our prediction error was similar to the work by Giollo et al. However, there were some key differences in the predictions by us and by Giollo et al. We included 15 additional PGP samples in our test dataset. For samples hu2DBF2D and hu52B7E5, we correctly predicted the ABO groups and they did not. Similarly, for sample huC30901, we correctly predicted the Rh group and they did not.

TABLE 21
Number of Prediction Errors vs Percentage of samples removed
from the training set. ABO, RhD and ABO + RhD prediction errors
versus the percentage of removed samples from the training set.
In general the trend if towards higher number of errors as the
percentage increases, however the error differences between 0%
and 50% is only 3, which means that the algorithm is quite robust
to changes in the training set.

| Training set | Number | Number | Number |
|---|---|---|---|
| removed | of Errors | of Errors | of Errors |
| (%) | ABO | RhD | ABO + RhD |
| 0 | 5 | 6 | 10 |
| 2.5 | 6 | 6 | 11 |
| 5 | 6 | 7 | 12 |
| 10 | 6 | 6 | 11 |
| 20 | 6 | 6 | 11 |
| 33 | 7 | 7 | 13 |
| 50 | 6 | 8 | 13 |

### Example 11-Metric Learning for Individual Identification

One goal of the examples provided herein is to identify individuals based on their genomes within a pool of N subjects with multiple phenotypes including 3D face, height, weight, and BMI. To this aim, we introduced a set of intermediate traits (e.g., ancestry, age, and gender) to bridge the gap between the genome and the 3D face. We predicted the intermediate traits from two sides. On the one side, we predicted them from the real faces for N subjects in the database and on the other side from the genome of interest that we want to match to the subjects in this database. Then, we determined the subject in the database that had the smallest distance between the two corresponding sets of predictions. Here, the distance on each individual trait (or dimension in the case of multidimensional traits) is defined as the absolute difference. In order to combine the distances for the set of all intermediate traits, we could in the simplest case just take the sum over these individual distances. However, in such a case, all intermediate traits would be treated equally. Ideally, more discriminative traits should be given higher weights in the combination. In this section, we present our metric learning approach to address the aforementioned problem, which significantly improves the identification performance.

The key idea is to learn and then utilize a measure of importance for each trait (or each dimension for multidimensional traits) when combining them. For illustration purposes, suppose that we want to identify the i-th individual's face from the i-th individual's genome among the pool of N faces. Our approach can be applied to any combination of any phenotypes. Specifically, we first predict ancestry, age, and gender from both the i-th individual's genome and N faces, referred to as {qᵢ} and {d₁ . . . d_{N}}, respectively. Here qᵢ and dⱼ, j=1, . . . , N are D-dimensional column vectors, where D is the dimension of all the intermediate traits of ancestry, age, and gender. Then we construct a matrix Xᵢ by taking the distances between each corresponding pair of predictions: Xᵢ=[|qᵢ−d₁|, . . . , |qᵢ−d_{N}|]. Now let us define the probability to choose the j-th face as the correct one among N faces as follows:

$$P ( j \vert {X}_{i} ) = \frac{exp {\sum }_{m = 1}^{D} {w}_{m} {X}_{mj}}{{\sum }_{k = 1}^{N} exp ( {\sum }_{m = 1}^{D} {w}_{m} {X}_{mk} )} ,$$

where wₘ represents the weight for the m-th feature, and Xₘⱼ represents the entry at the m-th row and j-th column in Xᵢ. We then maximize the log-likelihood of L=log Π_{I}P(i=jᵢ|Xᵢ) over the weights {wₘ}, where j is the index of the i-th individual's face in the pool of N faces. To maximize the log-likelihood L, we employed the YASMET software (www.fjoch.com/yasmet.html). After learning the weight {wₘ}, we selected the face with the largest P(j|Xᵢ) as the closest face to the i-th genome.

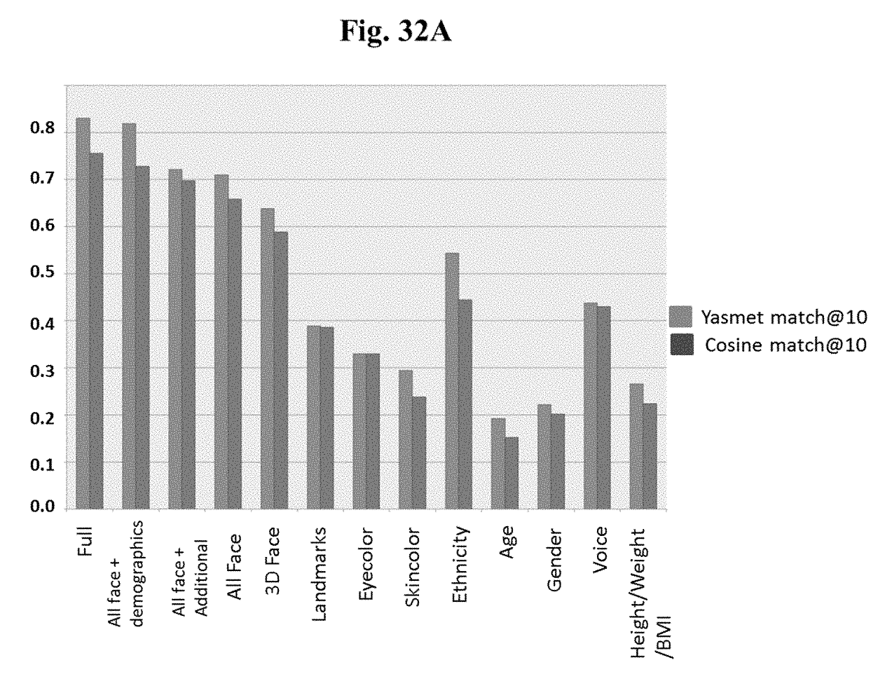

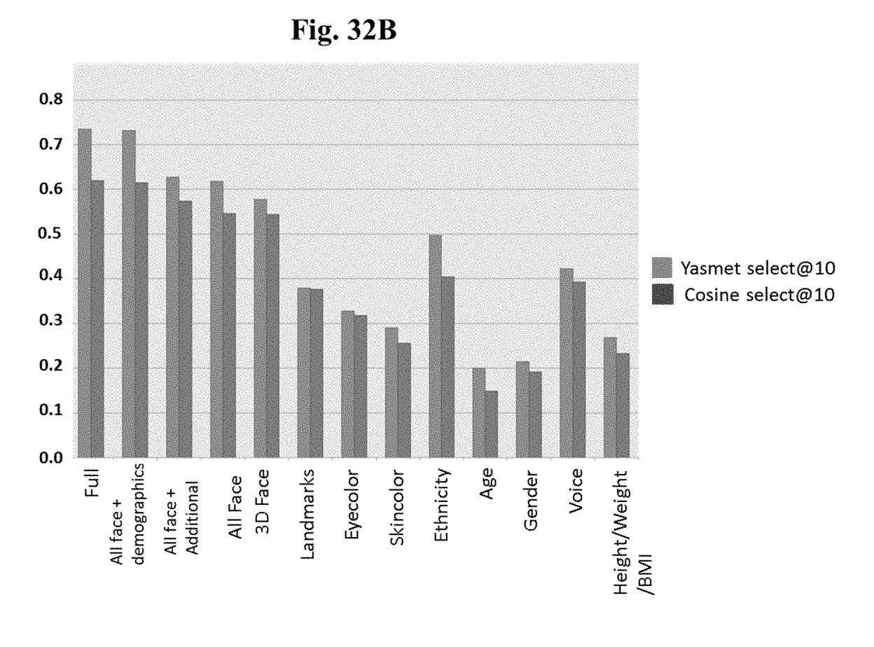

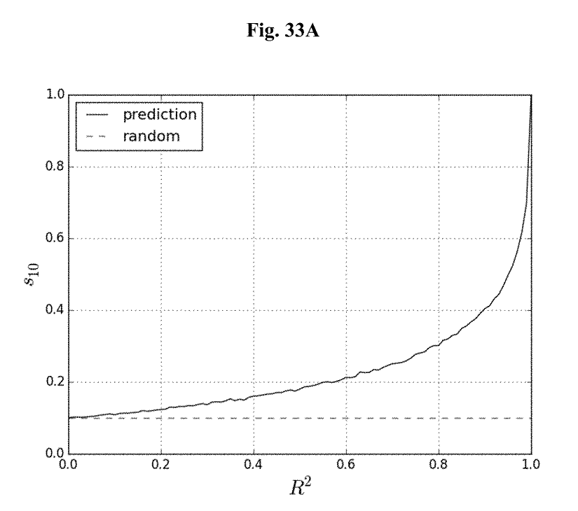

In FIGS. 32A and 32B, we show m₁₀ and s₁₀ using YASMET and cosine distance on different combination of phenotypes. We chose the cosine distance for comparison, where we found the closest face to the i-th genome by taking the maximum of {cosine(qᵢ,d₁), . . . , cosine(qᵢ,d_{N})}. As shown in the figures, for 25 out of 26 settings, YASMET showed better performance than cosine (binomial p-value<10⁻⁵). In particular, YASMET was significantly better than cosine by ˜10% in both m₁₀ and s₁₀ when using ancestry as the phenotype, where self-reported 5 ancestry and genome-inferred 5 ancestry were matched. It demonstrates that some ancestry components are more important than others for individual identification in our cohorts, and our metric learning approach properly adjusted the feature weights to achieve high identification performance.

### Select Performance Simulation

We simulated independent Gaussian distributed traits yᵢ for 1,000 individuals as the sum of a Gaussian distributed predictor pᵢ and an unpredictable Gaussian noise component ∈ᵢ:

yᵢ=pᵢ+∈ᵢ.

pᵢ˜N(0,R²).

∈ᵢ˜N(0,1−R²).

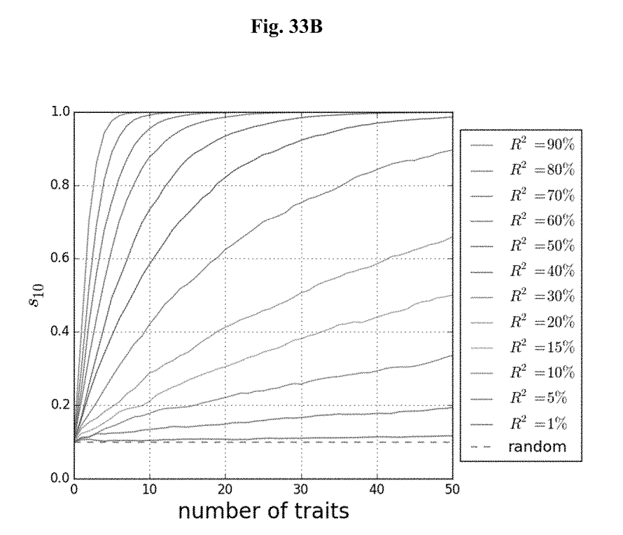

This way we achieve an expected variance explained of R²_{cv} for each trait. FIG. 33 shows how s₁₀ changes for a single trait that can be predicted at a given R²_{cv} between 0 and 1. FIG. 34 shows s₁₀ as a function of the number of traits that each can be predicted at a given expected R².

TABLE 22
Additional SNPs identified in the literature for eye color prediction and
tested for the prediction models.

| | |
|---|---|
| List A | rs10765198 rs10852218 rs11074304 rs11568820 rs11572177 |
| | rs11631195 rs11636232 rs12324648 rs12520016 rs12592307 |
| | rs1375164 rs1448481 rs1448490 rs1470608 rs1498509 |
| | rs1498519 rs1498521 rs1562592 rs1603784 rs17084733 |
| | rs17673969 rs17674017 rs1800404 rs1800410 rs1800411 |
| | rs1800416 rs1800419 rs1874835 rs1973448 rs2015343 |
| | rs2254913 rs2290100 rs2311843 rs2594902 rs2594938 |
| | rs2681092 rs2689229 rs2689230 rs2689234 rs2703922 |
| | rs2703969 rs2871875 rs3002288 rs3782974 rs4253231 |
| | rs4278697 rs4778137 rs4778177 rs4778185 rs4778190 |
| | rs4778220 rs4810147 rs6785780 rs7170989 rs7173419 |
| | rs7175046 rs7176632 rs7176759 rs728404 rs7643410 rs7975232 |
| | rs9476886 rs9584233 rs977588 rs977589 |
| List B | rs1042602 rs10765198 rs10852218 rs11074304 rs1126809 |
| | rs1129038 rs11568820 rs11572177 rs11631195 rs11636232 |
| | rs12203592 rs12324648 rs12520016 rs12592307 rs12896399 |
| | rs12913832 rs1375164 rs1393350 rs1408799 rs1448481 |
| | rs1448485 rs1448490 rs1470608 rs1498509 rs1498519 |
| | rs1498521 rs1540771 rs1562592 rs1597196 rs1603784 |
| | rs1667394 rs16891982 rs17084733 rs17673969 rs17674017 |
| | rs1800401 rs1800404 rs1800407 rs1800410 rs1800411 |
| | rs1800414 rs1800416 rs1800419 rs1805005 rs1874835 |
| | rs1973448 rs2015343 rs2238289 rs2254913 rs2290100 |
| | rs2311843 rs2594902 rs2594938 rs26722 rs2681092 rs2689229 |
| | rs2689230 rs2689234 rs2703922 rs2703969 rs2733832 |
| | rs2871875 rs3002288 rs3782974 rs3794604 rs4253231 |
| | rs4278697 rs4778137 rs4778138 rs4778177 rs4778185 |
| | rs4778190 rs4778220 rs4778232 rs4778241 rs4810147 |
| | rs6058017 rs6785780 rs683 rs7170852 rs7170989 rs7173419 |
| | rs7174027 rs7175046 rs7176632 rs7176759 rs7179994 |
| | rs7183877 rs728404 rs7495174 rs7643410 rs7975232 rs8024968 |
| | rs916977 rs9476886 rs9584233 rs977588 rs977589 |
| List C | rs10001971 rs10007810 rs1003719 rs10108270 rs1015362 |
| | rs10209564 rs10235789 rs10236187 rs1040045 rs1040404 |
| | rs1042602 rs10496971 rs10510228 rs10511828 rs10512572 |
| | rs10513300 rs1074265 rs10839880 rs10954737 rs1105879 |
| | rs1110400 rs11164669 rs11227699 rs1126809 rs1129038 |
| | rs11547464 rs11631797 rs11652805 rs12130799 rs12203592 |
| | rs12439433 rs12452184 rs12544346 rs12592730 rs12593929 |
| | rs12629908 rs12657828 rs12821256 rs1289399 rs12896399 |
| | rs12906280 rs12913823 rs12913832 rs1296819 rs1325127 |
| | rs1325502 rs13267109 rs13400937 rs1357582 rs1369093 |
| | rs1393350 rs1407434 rs1408799 rs1408801 rs1426654 rs143384 |
| | rs1448485 rs1471939 rs1500127 rs1503767 rs1510521 |
| | rs1513056 rs1513181 rs1533995 rs1540771 rs1569175 |
| | rs1597196 rs1635168 rs1667394 rs16891982 rs16950979 |
| | rs16950987 rs1760921 rs17793678 rs1800401 rs1800407 |
| | rs1800414 rs1805005 rs1805006 rs1805007 rs1805008 |
| | rs1805009 rs1837606 rs1871428 rs1879488 rs192655 rs1950993 |
| | rs199501 rs2001907 rs200354 rs2030763 rs2033111 rs2069398 |
| | rs2070586 rs2070959 rs2073730 rs2073821 rs2125345 rs214678 |
| | rs2228479 rs2238289 rs2240202 rs2240203 rs2252893 |
| | rs2269793 rs2277054 rs2278202 rs2306040 rs2330442 |
| | rs2346050 rs2357442 rs2397060 rs2416791 rs2424905 |
| | rs2424928 rs2504853 rs2532060 rs2594935 rs260690 rs2627037 |
| | rs26722 rs2702414 rs2709922 rs2724626 rs2733832 rs2835370 |
| | rs2835621 rs2835630 rs2899826 rs2946788 rs2966849 |
| | rs2986742 rs3118378 rs316598 rs316873 rs32314 rs35264875 |
| | rs35414 rs37369 rs3737576 rs3739070 rs3745099 rs3768056 |
| | rs3784230 rs3793451 rs3793791 rs3794604 rs3822601 |
| | rs3829241 rs385194 rs3935591 rs3940272 rs3943253 rs4458655 |
| | rs4463276 rs4530349 rs4666200 rs4670767 rs4673339 rs471360 |
| | rs4738909 rs4746136 rs4778138 rs4778232 rs4778241 |
| | rs4781011 rs4798812 rs4800105 rs4821004 rs4880436 |
| | rs4891825 rs4900109 rs4908343 rs4911414 rs4911442 |
| | rs4918842 rs4925108 rs4951629 rs4955316 rs4984913 rs507217 |
| | rs5768007 rs6058017 rs6104567 rs6422347 rs642742 rs6451722 |
| | rs6464211 rs647325 rs6493315 rs6541030 rs6548616 rs6556352 |
| | rs6759018 rs683 rs7029814 rs705308 rs7170852 rs7174027 |
| | rs7179994 rs7183877 rs7219915 rs7238445 rs7277820 rs728405 |
| | rs731257 rs734873 rs7421394 rs7495174 rs7554936 rs7657799 |
| | rs772262 rs7745461 rs7803075 rs7844723 rs798443 rs7997709 |
| | rs8021730 rs8024968 rs8028689 rs8035124 rs8041209 |
| | rs8113143 rs818386 rs870347 rs874299 rs881728 rs885479 |
| | rs892839 rs916977 rs9291090 rs9319336 rs946918 rs948028 |
| | rs9522149 rs9530435 rs9782955 rs9809104 rs9845457 |
| | rs9894429 rs989869 |

TABLE 23
List of replicated SNPs from previous human facial variation GWAS (80). We
have used a less stringent threshold of 1e-3 as we have fewer samples (870 on average)
than this study (6000 samples). P-value with * represent Bonferroni-adjusted p-values
reported by Adhikari et al. A total of 6 SNPs were replicated, which are associated with 6
facial phenotypes (brow ridge protrusion, Upper lip thickness, columella inclination, nose
protrusion, nose tip angle, nose wing breath). Bold marks opposite (L/R) results that are
above the reporting threshold of 1e-3.

| rsid | hr | position | Associated Trait | P-Value | Reported P-value (80) |
|---|---|---|---|---|---|
| rs2235371 | | 209790735 | SL_PG | 6.7e−4 | Brow ridge protrusion |
| | | | | | (0.0019*), |
| | | | | | Upper lip thickness |
| | | | | | (0.0066*) |
| rs2045323 | | 153910747 | AL_L_LI | 3.5e−4 | Columella inclination |
| | | | AL_L_SL | 9.5e−4 | (3e−9) |
| | | | AL_L_ST | 2.6e−4 | Nose protrusion |
| | | | AL_R_LI | 1.0e−4 | (1e−9) |
| | | | AL_R_SL | 2.1e−4 | Nose Tip Angle |
| | | | AL_R_ST | 4.3e−5 | (2e−8) |
| | | | CPH_R_STO | 4.2e−4 | |
| | | | CPH_L_STO | 1.0e−1 | |
| | | | SBAL_L_LI | 9.8e−4 | |
| | | | SBAL_R_LI | 2.9e−4 | |
| | | | SBAL_R_SL | 5.4e−4 | |
| | | | SBAL_R_STO | 7.5e−4 | |
| | | | SBAL_L_STO | 1.8e−1 | |
| rs12651681 | | 154328210 | EB_L_EN_L | 5.9e−5 | Columella inclination |
| | | | EB_L_IR_L | 1.1e−4 | (2.4e−8) |
| | | | EB_R_IR_R | 3.8e−3 | |
| | | | EB_L_PI_L | 8.5e−4 | |
| | | | EB_L_PI_L | 6.3e−3 | |
| | | | EB_R_EN_R | 3.4e−4 | |
| rs12644248 | | 154314240 | SBAL_L_PG | 3.4e−4 | Columella inclination |
| | | | SBAL_R_PG | 1.3e−3 | (6.6e−9) |
| rs12543318 | | 87856112 | CPH_R_CH_L | 7.8e−4 | Brow ridge protrusion |
| | | | | | (0.028*), |
| | | | CPH_L_CH_R | 3.4e−1 | Columella inclination |
| | | | | | (0.015*) |
| rs927833 | 0 | 22060939 | AL_L_LI | 3.4e−4 | Nose wing breath (1e−9) |
| | | | AL_L_SL | 7.2e−4 | |
| | | | AL_R_LI | 2.1e−4 | |
| | | | AL_R_SL | 6.8e−4 | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

1. A method of determining a facial structure of an individual from a nucleic acid sequence for the individual, the method comprising:
a) determining a plurality of genomic principal components from the nucleic acid sequence of the individual that are predictive of facial structure; and
b) determining at least one demographic feature from the nucleic acid sequence of the individual selected from the list consisting of:
i) an age of the individual;
ii) a sex of the individual; and
iii) an ancestry of the individual;
wherein the facial structure is determined according to the genomic principal components and the at least one demographic feature from the nucleic acid sequence of the individual.

2. The method of claim 1, wherein the facial structure of the individual is uncertain or unknown at the time of determination.

3. (canceled)

4. The method of claim 1, wherein the genomic principal components are derived from a data set comprising a plurality of facial structure measurements and a plurality of genome sequences.

5. (canceled)

6. The method of claim 4, wherein the genomic principal components from the nucleic acid sequence that are predictive of facial structure are predictive of facial landmark distance.

7. (canceled)

8. The method of claim 1, wherein the nucleic acid sequence for the individual is an in silico generated sequence that is a composite of two individuals.

9. (canceled)

10. The method of claim 1, wherein the plurality of genomic principal components determine at least 90% of the observed variation of facial structure.

11. The method of claim 1, wherein the age of the individual is determined by both an average telomere length and a mosaic loss of a sex chromosome from the nucleic acid sequence for the individual.

12. (canceled)

13. The method of claim 12, wherein the average telomere length is determined by a proportion of putative telomere reads to total reads.

14. (canceled)

15. The method of claim 14, wherein the mosaic loss of Y chromosome is determined by sequences from the Y chromosome that are Y chromosome specific.

16. (canceled)

17. The method of claim 11, wherein the mosaic loss of the sex chromosome is determined by determining chromosomal copy number.

18. (canceled)

19. The method of claim 1, wherein the mean absolute error of the determined age of the individual is equal to or less than 10 years.

20. The method claim 1, wherein the R²_{CV} of the determined age of the individual is equal to or greater than 0.40.

21. The method of claim 1, wherein the sex of the individual is determined by estimating copy number of the X and Y chromosome.

22. (canceled)

23. The method of claim 1, wherein the ancestry of the individual is determined by a plurality of single nucleotide polymorphisms that are informative of ancestry.

24. (canceled)

25. The method of claim 1, wherein the nucleic acid sequence for the individual was obtained from a biological sample, the method further comprising determining a body mass index of the individual from the biological sample.

26. The method of claim 1, further comprising determining the presence or absence of at least one single nucleotide polymorphism associated with facial structure.

27. The method of claim 1, wherein the determined facial structure is represented by a plurality of land mark distances.

28. The method of claim 27, wherein the plurality of land mark distances comprise at least two or more of TGL_TGRpa, TR_GNpa, EXR_ENR (Width of the right eye), PSR_PIR (Height of the right eye), ENR_ENL (Distance from inner left eye to inner right eye), EXL_ENL (Width of the left eye), EXR_EXL (Distance from outer left eye to outer right eye), PSL_PIL (Height of the left eye), ALL_ALR (Width of the nose), N_SN (Height of the nose), N_LS (Distance from top of the nose to top of upper lip), N_ST (Distance from top of the nose to center point between lips), TGL_TGR (Straight distance from left ear to right ear), EBR_EBL (Distance from inner right eyebrow to inner left eyebrow), IRR_IRL (Distance from right iris to left iris), SBALL_SBALR (Width of the bottom of the nose), PRN_IRR (Distance from the tip of the nose to right iris), PRN_IRL (Distance from the tip of the nose to left iris), CPHR_CPHL (Distance separating the crests of the upper lip), CHR_CHL (Width of the mouth), LS_LI (Height of lips), LS_ST (Height of upper lip), LI_ST (Height of lower lip), TR_G (Height of forehead), SN_LS (Distance from bottom of the nose to top of upper lip), LI_PG (Distance from bottom of the lower lip to the chin).

29. The method of claim 27, wherein the plurality of land mark distances comprise ALL_ALR (width of nose) and LS_LI (height of lip).

30. (canceled)

31. (canceled)

32. (canceled)

33. A computer-implemented system comprising a digital processing device comprising at least one processor, an operating system configured to perform executable instructions, a memory, and a computer program including instructions executable by the digital processing device to create an application comprising:
a) a software module for determining a plurality of genomic principal components from the nucleic acid sequence of an individual that are predictive of facial structure;
b) a software module for determining at least one demographic feature from the nucleic acid sequence of the individual, the demographic feature selected from the list consisting of:
i) an age of the individual;
ii) a sex of the individual; and
iii) an ancestry; and
c) a software module generating a graphical representation of a facial structure of the individual on a computer display according to the genomic principal components and the at least one demographic feature from the nucleic acid sequence of the individual.
